(19) United States
(12) Patent Application Publication
(10) Pub. No.: US 2024/0252181 A1
(43) Pub. Date: Aug. 1, 2024

(54) APPARATUS, SYSTEM, AND METHOD FOR INSTRUMENTATION

(71) Applicant: Treace Medical Concepts, Inc., Ponte Vedra, FL (US)

(72) Inventors: Adam D. PERLER, St. Petersburg, FL (US); James Q. SPITLER, Winter Garden, FL (US)

(21) Appl. No.: 18/427,593

(22) Filed: Jan. 30, 2024

Related U.S. Application Data
(60) Provisional application No. 63/482,197, filed on Jan. 30, 2023.

(51) Int. Cl. A61B 17/15; A61B 17/00; A61B 17/17; A61B 17/56

(52) U.S. Cl. CPC A61B 17/151; A61B 17/1775; A61B 2017/00982; A61B 2017/564

(57) ABSTRACT

An apparatus, system, and method are disclosed for remediating a condition present in a patient. In certain implementations, the device may include a body having a medial side, a lateral side, a superior side, an inferior side, an anterior side, and a posterior side. In addition, the device may include a bone engagement surface on a bone-facing side of the body, the bone engagement surface configured to engage a surface of a patient. The device may include a bone attachment feature configured to couple the body to a long bone of the patient. Moreover, the device may include a resection feature that guides resection of the long bone to separate a malleolus from the long bone.

### CROSS-REFERENCE TO RELATED APPLICATIONS

This application claims the benefit of and priority to U.S. Provisional Application No. 63/482,197, filed Jan. 30, 2023, which is hereby incorporated by reference in its entirety.

### TECHNICAL FIELD

The present disclosure relates to surgical devices, systems, instruments, and methods. More specifically, the present disclosure relates to patient-specific guides, implants, instruments, and/or methods of designing and using the same.

### BACKGROUND

Various bone conditions may be corrected using surgical procedures, in which one or more tendons, ligaments, and/or bones may be cut, replaced, repositioned, reoriented, reattached, fixated and/or fused. These surgical procedures require the surgeon to properly locate, position, and/or orient one or more osteotomy cuts, fixation guides, fixators, bone tunnels, points of attachment for ends of grafts or soft tissue and the like. Determining and locating an optimal location and trajectory for one or more steps of the surgical procedures can be challenging, given conventional techniques and instruments.

In certain circumstances, a surgeon may need to perform an osteotomy in order to provide access to another part of a patient. For example, a surgeon may plan to operate on a talus of a patient. However, in order to gain access for work on the talus a surgeon may dissect a malleolus (medial or lateral) and temporarily move the malleolus to provide access for surgery on the talus. However, conventional approaches and/or instrumentation are a challenge to properly place and use to make cuts to separate the malleolus from the bone and provide suitable reduction and/or fixation for the malleolus once access to the talus is no longer needed.

What is needed is one or more guides to facilitate locating, aligning, orienting, planning, preparing for, initiating, and/or completing an osteotomy, such as an osteotomy of a malleolus. Existing solutions for guiding orthopedic surgical procedures are inadequate and error prone.

### SUMMARY

The various apparatus, devices, systems, and/or methods of the present disclosure have been developed in response to the present state of the art, and in particular, in response to the problems and needs in the art that have not yet been fully solved by currently available technology.

In one general aspect, the apparatus may include a body having a medial side, a lateral side, a superior side, an inferior side, an anterior side, and a posterior side. The apparatus may also include a bone engagement surface on a bone-facing side of the body, the bone engagement surface configured to engage a surface of a patient. The apparatus may furthermore include a bone attachment feature configured to couple the body to a long bone of the patient. The apparatus may in addition include a resection feature that guides resection of the long bone to separate a malleolus from the long bone.

Implementations may include one or more of the following features. An apparatus where the surface of the patient is a surface of the long bone and the bone engagement surface is shaped to match a contour of the surface of the long bone and defined at least in part based on medical imaging of a portion of the long bone. An apparatus where the bone engagement surface is defined at least in part based on a bone model of a portion of the long bone.

An apparatus where: the surface of the patient is a surface of skin covering the long bone of the patient; and the resection feature is configured to guide resection of the skin by a cutting tool to expose the long bone. An apparatus where the resection feature may include an opening that extends from the bone-facing side to a side opposite the bone-facing side. An apparatus where the opening of the resection feature extends through the body at a patient-specific angle defined at least in part based on medical imaging of a portion of the long bone of the patient.

An apparatus may include a patient-specific trajectory guide. An apparatus may include a patient-matched trajectory guide. An apparatus where the bone attachment feature may include one or more holes that extend through the body from one side of the body to the bone-facing side of the body. An apparatus where the one or more holes extend through the body at a first trajectory that substantially matches a second trajectory of an opening of the resection feature.

An apparatus where the one or more holes have a circular cross section and a first diameter substantially the same as a second diameter of one or more anchor pins configured for deployment through the one or more holes to secure the body to the long bone. An apparatus may include a stop configured to control a maximum depth of a cutting tool inserted into the resection feature. An apparatus may include a fixation guide having one or more openings that indicate a path for fasteners into the long bone.

In one general aspect, the osteotomy system may include a resection guide having: one or more bone attachment features configured to couple the resection guide to a bone; and a resection feature that guides resection of the bone to dissect the bone into a proximal fragment and distal fragment. The osteotomy system may also include a fixation guide having one or more openings that indicate a path for fasteners through the distal fragment and into the proximal fragment of the bone.

Implementations may include one or more of the following features. An osteotomy system where the one or more bone attachment features align with the resection feature and one or more of the resection guide and the fixation guide may include a bone engagement surface. An osteotomy system where the fixation guide may include one or more sleeves each configured to accept a fastener.

An osteotomy system may include a body having a coupler configured to couple the resection guide to the fixation guide and position the fixation guide relative to the resection guide. An osteotomy system where the one or more openings of the fixation guide are aligned vertically with respect to a longitudinal axis of the bone. An osteotomy system where the one or more openings of the fixation guide extend through the fixation guide at a patient-specific angle.

In one general aspect, the method may include anchoring a resection guide to a surface of a long bone and proximal to a malleolus of the long bone by way of one or more anchor pins, the resection guide having a bone engagement surface configured to register to the surface of the long bone. The method may also include connecting one or more pin guide sleeves to a fixation guide coupled to the resection guide. The method may furthermore include deploying one or more guide pins through the pin guide sleeves and into the long bone.

The method may in addition include drilling one or more openings coaxial with the one or more guide pins. The method may moreover include deploying fasteners coaxial with the openings. The method may also include removing the fasteners. The method may furthermore include dissecting an osteotomy fragment from the long bone. The method may in addition include removing the resection guide from the long bone.

The method may moreover include retracting the osteotomy fragment inferiorly from the long bone to expose a portion of a talus, the osteotomy fragment including the malleolus. The method may also include performing a remediation procedure on the talus. The method may furthermore include reducing the osteotomy fragment with the long bone. The method may in addition include redeploying one or more guide pins into the one or more openings of the osteotomy fragment and the long bone. The method may moreover include deploying fasteners coaxial with the one or more openings to fix the osteotomy fragment to the long bone.

### BRIEF DESCRIPTION OF THE DRAWINGS

The advantages, nature, and additional features of exemplary embodiments of the disclosure will become more fully apparent from the following description and appended claims, taken in conjunction with the accompanying drawings. Understanding that these drawings depict only exemplary embodiments and are, therefore, not to be considered limiting of the disclosure's scope, the exemplary embodiments of the disclosure will be described with additional specificity and detail through use of the accompanying drawings.

FIG. 1A is a flowchart diagram depicting a method for remediating a condition, according to one embodiment.

FIG. 1B is a flowchart diagram depicting a method for remediating a condition, according to one embodiment.

FIG. 2A is a dorsal perspective view of bones of a foot.

FIG. 2B is a lateral perspective view of bones of a foot.

FIG. 2C is a lateral perspective view of bones of a foot.

FIG. 2D is a dorsal perspective view of bones of a foot.

FIG. 2E is a view of a foot illustrating common planes of reference for a human foot.

FIG. 3 is a flowchart diagram depicting a method for generating one or more patient-specific instruments configured to address a bone condition, according to one embodiment.

FIG. 4 illustrates an exemplary system configured to generate one or more patient-specific instruments configured to address a bone condition, according to one embodiment.

FIG. 5 illustrates an exemplary apparatus configured according to one embodiment.

FIG. 6 illustrates an exemplary provision module configured to provide a preliminary guide model, according to one embodiment.

FIG. 7 illustrates an exemplary design module configured to design a patient-specific guide model, according to one embodiment.

FIG. 8 illustrates an exemplary system configured to generate one or more patient-specific instruments configured to address a bone condition, according to one embodiment.

FIG. 9 illustrates an exemplary system, according to one embodiment.

FIG. 10 illustrates an exemplary guide system for a surgical procedure, according to one embodiment.

FIGS. 11A-11D illustrate views of an example guide, according to one embodiment.

FIG. 11E illustrates a guide according to one embodiment.

FIG. 11F illustrates a guide according to one embodiment.

FIG. 11G illustrates a guide according to one embodiment and example fixation hardware that can be used with the guide.

FIG. 11H illustrates a variety of example fixation hardware that can be used with embodiments of guides of the present disclosure.

FIGS. 12A-12D illustrate views of an example guide, according to one embodiment.

FIGS. 13A-13F illustrate different views of stages of a surgical procedure.

FIG. 14 illustrates a method for resecting and/or providing fixation for a bone portion to a tibia, according to one embodiment.

### DETAILED DESCRIPTION

Exemplary embodiments of the disclosure will be best understood by reference to the drawings, wherein like parts are designated by like numerals throughout. It will be readily understood that the components, as generally described and illustrated in the Figures herein, could be arranged and designed in a wide variety of different configurations. Thus, the following more detailed description of the embodiments of the apparatus, system, and method is not intended to limit the scope of the disclosure but is merely representative of exemplary embodiments.

The phrases “connected to,” “coupled to” and “in communication with” refer to any form of interaction between two or more entities, including mechanical, electrical, magnetic, electromagnetic, fluid, and thermal interaction. Two components may be functionally coupled to each other even though they are not in direct contact with each other. The term “abutting” refers to items that are in direct physical contact with each other, although the items may not necessarily be attached together. The phrase “fluid communication” refers to two features that are connected such that a fluid within one feature can pass into the other feature.

As used herein, “coupling”, “coupling member”, or “coupler” refers to a mechanical device, apparatus, member, component, system, assembly, or structure, that is organized, configured, designed, arranged, or engineered to connect, or facilitate the connection of, two or more parts, objects, or structures. In certain embodiments, a coupling can connect adjacent parts or objects at their ends. In certain embodiments, a coupling can be used to connect two shafts together at their ends for the purpose of transmitting power. In other embodiments, a coupling can be used to join two pieces of rotating equipment while permitting some degree of misalignment or end movement or both. In certain embodiments, couplings may not allow disconnection of the two parts, such as shafts during operation. (Search “coupling” on Wikipedia.com Jul. 26, 2021. CC-BY-SA 3.0 Modified. Accessed Jul. 27, 2021.) A coupler may be flexible, semiflexible, pliable, elastic, or rigid. A coupler may join two structures either directly by connecting directly to one structure and/or directly to the other or indirectly by connecting indirectly (by way of one or more intermediary structures) to one structure, to the other structure, or to both structures.

The word “exemplary” is used herein to mean “serving as an example, instance, or illustration.” Any embodiment described herein as “exemplary” is not necessarily to be construed as preferred or advantageous over other embodiments. While the various aspects of the embodiments are presented in drawings, the drawings are not necessarily drawn to scale unless specifically indicated.

Standard medical planes of reference and descriptive terminology are employed in this disclosure. While these terms are commonly used to refer to the human body, certain terms are applicable to physical objects in general. A standard system of three mutually perpendicular reference planes is employed. A sagittal plane divides a body into right and left portions. A coronal plane divides a body into anterior and posterior portions. A transverse plane divides a body into superior and inferior portions. A mid-sagittal, mid-coronal, or mid-transverse plane divides a body into equal portions, which may be bilaterally symmetric. The intersection of the sagittal and coronal planes defines a superior-inferior or cephalad-caudal axis. The intersection of the sagittal and transverse planes defines an anterior-posterior axis. The intersection of the coronal and transverse planes defines a medial-lateral axis. The superior-inferior or cephalad-caudal axis, the anterior-posterior axis, and the medial-lateral axis are mutually perpendicular.

Anterior means toward the front of a body. Posterior means toward the back of a body. Superior or cephalad means toward the head. Inferior or caudal means toward the feet or tail. Medial means toward the midline of a body, particularly toward a plane of bilateral symmetry of the body. Lateral means away from the midline of a body or away from a plane of bilateral symmetry of the body. Axial means toward a central axis of a body. Abaxial means away from a central axis of a body. Ipsilateral means on the same side of the body. Contralateral means on the opposite side of the body from the side which has a particular condition or structure. Proximal means toward the trunk of the body. Proximal may also mean toward a user, viewer, or operator. Distal means away from the trunk. Distal may also mean away from a user, viewer, or operator. Dorsal means toward the top of the foot or other body structure. Plantar means toward the sole of the foot or toward the bottom of the body structure.

Antegrade means forward moving from a proximal location/position to a distal location/position or moving in a forward direction. Retrograde means backward moving from a distal location/position to a proximal location/position or moving in a backwards direction. Sagittal refers to a midline of a patient's anatomy, which divides the body into left or right halves. The sagittal plane may be in the center of the body, splitting it into two halves. Prone means a body of a person lying face down. Supine means a body of a person lying face up.

“Patient specific” refers to a feature, an attribute, a characteristic, a structure, function, structure, device, guide, tool, instrument, apparatus, member, component, system, assembly, module, or subsystem or the like that is adjusted, tailored, modified, organized, configured, designed, arranged, engineered, and/or fabricated to specifically address the anatomy, physiology, condition, abnormalities, needs, or desires of a particular patient or surgeon serving the particular patient. In one aspect, a patient specific attribute or feature is unique to a single patient and may include features unique to the patient such as a number of cut channels, a number of bone attachment features, a number of bone engagement surfaces, a number of resection features, a depth of one or more cutting channels, an angle for one or more resection channels, a surface contour, component position, component orientation, a trajectory for an instrument, implant, or anatomical part of a patient, a lateral offset, and/or other features.

“Patient-specific guide” refers to a guide designed, engineered, and/or fabricated for use with a specific patient. In one aspect, a patient-specific guide is unique to a patient and may include features unique to the patient such as a surface contour or other features.

“Patient-specific cutting guide” refers to a cutting guide designed, engineered, and/or fabricated for use with a specific patient. In one aspect, a patient-specific cutting guide is unique to a patient and may include features unique to the patient such as a surface contour or other features.

“Patient-specific resection guide” refers to a guide designed, engineered, and/or fabricated for use in resection for a specific patient. In one aspect, a patient-specific resection guide is unique to a patient and may include features unique to the patient such as a surface contour or other features.

“Patient-specific angle” refers to an angle that is patient-specific.

“Patient-matched” refers to a feature, aspect, attribute, characteristic, instrument, and/or device that is selected from a set of predetermined, predefined, precalculated, preconfigured, prearranged, and/or pre-fabricated structures, apparatuses, devices, instruments or devices to satisfactorily service a user based on a set of characteristics, such as size of an anatomical structure, deformity, fracture, laceration, opening, angles for certain landmarks, angles for a deformity, type of deformity, size of the bone, and the like. In certain embodiments, patient-matched is different from patient-specific.

As used herein, “implant” refers to a medical device manufactured to replace a missing biological structure, support a damaged biological structure, or enhance an existing biological structure. Often medical implants are man-made devices, but implants can also be natural occurring structures. The surface of implants that contact the body may be made of, or include a biomedical material such as titanium, cobalt chrome, stainless steel, carbon fiber, another metallic alloy, silicone, polymer, Synthetic polyvinyl alcohol (PVA) hydrogels, biomaterials, biocompatible polymers such as PolyEther Ether Ketone (PEEK) or a polylactide polymer (e.g. PLLA) and/or others, or apatite, or any combination of these depending on what is functional and/or economical. Implants can have a variety of configurations and can be wholly, partially, and/or include a number of components that are flexible, semiflexible, pliable, elastic, supple, semi-rigid, or rigid. In some cases implants contain electronics, e.g. artificial pacemaker and cochlear implants. Some implants are bioactive, such as subcutaneous drug delivery devices in the form of implantable pills or drug-eluting stents. Orthopedic implants may be used to alleviate issues with bones and/or joints of a patient's body. Orthopedic implants can be used to treat bone fractures, osteoarthritis, scoliosis, spinal stenosis, discomfort, and pain. Examples of orthopedic implants include, but are not limited to, a wide variety of pins, rods, screws, anchors, spacers, sutures, all-suture implants, ball all-suture implants, self-locking suture implants, cross-threaded suture implants, plates used to anchor fractured bones while the bones heal or fuse together, and the like. (Search “implant (medicine)” on Wikipedia.com May 26, 2021. CC-BY-SA 3.0 Modified. Accessed Jun. 30, 2021.)

As used herein, a “body” refers to a main or central part of a structure. The body may serve as a structural component to connect, interconnect, surround, enclose, and/or protect one or more other structural components. A body may be made from a variety of materials including, but not limited to, metal, plastic, ceramic, wood, fiberglass, acrylic, carbon, biocompatible materials, biodegradable materials or the like. A body may be formed of any biocompatible materials, including but not limited to biocompatible metals such as Titanium, Titanium alloys, stainless steel alloys, cobalt-chromium steel alloys, nickel-titanium alloys, shape memory alloys such as Nitinol, biocompatible ceramics, and biocompatible polymers such as Polyether ether ketone (PEEK) or a polylactide polymer (e.g. PLLA) and/or others. In one embodiment, a body may include a housing or frame or framework for a larger system, component, structure, or device. A body may include a modifier that identifies a particular function, location, orientation, operation, and/or a particular structure relating to the body. Examples of such modifiers applied to a body, include, but are not limited to, “inferior body,” “superior body,” “lateral body,” “medial body,” and the like.

As used herein, “bone engagement surface” refers to a surface of an object, instrument, or apparatus, such as an implant that is oriented toward or faces one or more bones of a patient. In one aspect, the bone engagement surface may abut, touch, or contact a surface of a bone. In another aspect, the bone engagement surface or parts of the bone engagement surface may be close to, but not abut, touch, or contact a surface of the bone. In certain aspects, the bone engagement surface can be configured to engage with a surface of one or more bones. Such a bone engagement surface may include projections and recesses that correspond to and match projections and recesses of the one or more bone surfaces.

As used herein, a “deploy” or “deployment” refers to an act, action, process, system, method, means, or apparatus for inserting an implant or prosthesis into a part, body part, and/or patient. “Deploy” or “deployment” can also refer to an act, action, process, system, method, means, or apparatus for placing something into therapeutic use. A device, system, component, medication, drug, compound, or nutrient may be deployed by a human operator, a mechanical device, an automated system, a computer system or program, a robotic system, or the like.

“Joint” or “Articulation” refers to the connection made between bones in a human or animal body which link the skeletal system to form a functional whole. Joints may be biomechanically classified as a simple joint, a compound joint, or a complex joint. Joints may be classified anatomically into groups such as joints of hand, elbow joints, wrist joints, axillary joints, sternoclavicular joints, vertebral articulations, temporomandibular joints, sacroiliac joints, hip joints, knee joints, articulations of foot, and the like. (Search “joint” on Wikipedia.com Dec. 19, 2021. CC-BY-SA 3.0 Modified. Accessed Jan. 20, 2022.)

“Topographical” refers to the physical distribution of parts, structures, or features on the surface of, or within, an organ or other anatomical structure, or organism. (Search “define topographical” on google.com. Oxford Languages, Copyright 2022. Oxford University Press. Web., Modified. Accessed 15 Feb. 2022.)

“Landmark registration features” or “Landmark feature” refers to a structure configured to engage with a feature, aspect, attribute, or characteristic of a first object to orient and/or position a second object that includes the landmark registration feature with respect to the first object. Often the first object is an anatomical landmark. A variety of structures can serve as a landmark registration feature. For example, a landmark registration feature may include a protrusion, a projection, a tuberosity, a cavity, a void, a divot, a tab, an extension, a hook, a curve, or the like. In the context of bones of a patient a landmark registration feature can include any protuberance, void, divot, concave section, sesamoid, bone spur or other feature on, or extending from, a bone of a patient.

“Landmark” refers to a structure on, in, or around a structure that can be used to serve as a reference for positioning, orienting, translating, rotating, or otherwise manipulating a second object or structure. For example, a landmark may include a protrusion, a projection, a tuberosity, a cavity, a void, a divot, a tab, an extension, a hook, a curve, or the like. In the context of bones of a patient, a landmark can include any protuberance, eminence, bony topography, anatomical features, calcifications, void, divot, concave section, sesamoid, bone spur or other feature on, or extending from, a bone of a patient. A landmark refers to any structure of an anatomical structure that is referenced, contacted, engaged with and/or associated with a landmark registration feature. In certain embodiments, a landmark is unique to one patient.

“Bone attachment feature” refers to a structure, feature, component, aspect configured to securely connect, couple, attach, and/or engage a structure, component, object, or body with a bone and/or a bone fragment. Examples of a bone attachment feature, include, but are not limited to, a pin, K-wire, screw, or other fastener alone, or in combination with, a hole, passage, and/or opening.

As used herein, a “stop” refers to an apparatus, instrument, structure, member, device, component, system, or assembly structured, organized, configured, designed, arranged, or engineered to prevent, limit, impede, stop, or restrict motion or movement and/or operation of the another object, member, structure, component, part, apparatus, system, or assembly.

As used herein, a “fastener”, “fixation device”, or “fastener system” refers to any structure configured, designed, or engineered to join two structures. Fasteners may be made of a variety of materials including metal, plastic, composite materials, metal alloys, plastic composites, and the like. Examples of fasteners include, but are not limited to screws, rivets, bolts, nails, snaps, hook and loop, set screws, bone screws, nuts, posts, pins, thumb screws, and the like. Other examples of fasteners include, but are not limited to wires, Kirschner wires (K-wire), anchors, bone anchors, plates, bone plates, intramedullary nails or rods or pins, implants, sutures, soft sutures, soft anchors, tethers, interbody cages, fusion cages, and the like.

In certain embodiments, the term fastener may refer to a fastener system that includes two or more structures configured to combine to serve as a fastener. An example of a fastener system is a rod or shaft having external threads and an opening or bore within another structure having corresponding internal threads configured to engage the external threads of the rod or shaft.

In certain embodiments, the term fastener may be used with an adjective that identifies an object or structure that the fastener may be particularly configured, designed, or engineered to engage, connect to, join, contact, or couple together with one or more other structures of the same or different types. For example, a “bone fastener” may refer to an apparatus for joining or connecting one or more bones, one or more bone portions, soft tissue and a bone or bone portion, hard tissue and a bone or bone portion, an apparatus and a bone or portion of bone, or the like.

In certain embodiments, a fastener may be a temporary fastener. A temporary fastener is configured to engage and serve a fastening function for a relatively short period of time. Typically, a temporary fastener is configured to be used until another procedure or operation is completed and/or until a particular event. In certain embodiments, a user may remove or disengage a temporary fastener. Alternatively, or in addition, another structure, event, or machine may cause the temporary fastener to become disengaged.

As used herein, “bone-facing side” refers to a side of an object, structure, instrument, or apparatus, such as an implant or instrument that is oriented toward or faces one or more bones of a patient. In one aspect, the bone-facing side may abut, touch, or contact a surface of a bone. In another aspect, the bone-facing side or parts of the bone-facing side may be close to, but not abut, touch, or contact a surface of the bone.

As used herein, an “opening” refers to a gap, a hole, an aperture, a port, a portal, a slit, a space or recess in a structure, a void in a structure, or the like. In certain embodiments, an opening can refer to a structure configured specifically for receiving something and/or for allowing access. In certain embodiments, an opening can pass through a structure. In such embodiments, the opening can be referred to as a window. In other embodiments, an opening can exist within a structure but not pass through the structure. In other embodiments, an opening can initiate on a surface or at an edge or at a side of a structure and extend into the structure for a distance, but not pass through or extend to another side or edge of the structure. In other embodiments, an opening can initiate on a surface or at an edge or at a side of a structure and extend into the structure until the opening extends through or extends to another side or edge of the structure. An opening can be two-dimensional or three-dimensional and can have a variety of geometric shapes and/or cross-sectional shapes, including, but not limited to a rectangle, a square, or other polygon, as well as a circle, an ellipse, an ovoid, or other circular or semi-circular shape. As used herein, the term “opening” can include one or more modifiers that define specific types of “openings” based on the purpose, function, operation, position, or location of the “opening ” As one example, a “fastener opening” refers to an “opening” adapted, configured, designed, or engineered to accept or accommodate a “fastener.”

“Hole” refers to a gap, an opening, an aperture, a port, a portal, a space or recess in a structure, a void in a structure, or the like. In certain embodiments, a hole can refer to a structure configured specifically for receiving something and/or for allowing access. In certain embodiments, a hole can pass through a structure. In other embodiments, an opening can exist within a structure but not pass through the structure. A hole can be two-dimensional or three-dimensional and can have a variety of geometric shapes and/or cross-sectional shapes, including, but not limited to a rectangle, a square, or other polygon, as well as a circle, an ellipse, an ovoid, or other circular or semi-circular shape. As used herein, the term “hole” can include one or more modifiers that define specific types of “holes” based on the purpose, function, operation, position, or location of the “hole.” As one example, a “fastener hole” refers to an “hole” adapted, configured, designed, or engineered to accept or accommodate a “fastener.” A “blind hole” is a hole with an opening on one side that does not extend all the way through a structure. In certain embodiments, a hole, including a blind hole, has a circular longitudinal cross-section. Alternatively, or in addition, a hole can have a cross-section of a variety of geometric shapes include a circle, an oval, a square, a rectangle, a slot with rounded ends, a triangle, or the like.

As used herein, a “fixator” refers to an apparatus, instrument, structure, device, component, member, system, assembly, or module structured, organized, configured, designed, arranged, or engineered to connect two bones or bone fragments or a single bone or bone fragment and another fixator to position and retain the bone or bone fragments in a desired position and/or orientation. Examples of fixators include both those for external fixation as well as those for internal fixation and include, but are not limited to pins, wires, Kirschner wires, screws, anchors, bone anchors, plates, bone plates, intramedullary nails or rods or pins, implants, interbody cages, fusion cages, and the like.

As used herein, an “anchor” refers to an apparatus, instrument, structure, member, part, device, component, system, or assembly structured, organized, configured, designed, arranged, or engineered to secure, retain, stop, and/or hold, an object to or at a fixed point, position, or location. An anchor may be coupled and/or connected to a flexible member such as a tether, chain, rope, wire, thread, suture, suture tape, or other like object. Alternatively, or in addition, an anchor may also be coupled, connected, and/or joined to a rigid object or structure. In certain embodiments, an anchor can be a fixation device. Said another way, a fixation device can function as an anchor. For example, an anchor pin is a pin, fastener, or K-wire that cooperates with a rigid structure to provide an anchor.

“Connector” refers to any structure configured, engineered, designed, adapted, and/or arranged to connect one structure, component, element, or apparatus to another structure, component, element, or apparatus. A connector can be rigid, pliable, elastic, flexible, and/or semiflexible. Examples of a connector include but are not limited any fastener.

As used herein, a “sleeve” refers to structure that is narrow and longer longitudinally than the structure is wide. In certain embodiments, a sleeve serves to surround, enclose, wrap, and/or contain something else. In certain embodiments, a sleeve may surround, enclose, wrap, and/or contain a passage or void. (Search “sleeve” on wordhippo.com. WordHippo, 2021. Web. Accessed 15 Nov. 2021. Modified.) In certain embodiments, the term sleeve may be preceded by an adjective that identifies the structure, implement, component or instrument that may be used with, inserted into or associated with the sleeve. For example, a “pin sleeve” may be configured to accept a pin or wire such as a K-wire, a “drive sleeve” may be configured to accept a drill or drill bit, a “fixation member sleeve” may be configured to accept a fastener or fixation member. A “pin guide sleeve” is a sleeve for a pin guide. The sleeve guides a pin. “Pin guide” refers to pin, or wire such as a K-wire, that serves as a guide for a fastener (e.g., a cannulated fastener).

As used herein, a “long bone” refers to a bone of a patient having a length greater than a width of the bone. Long bone is one of five types of bones: long, short, flat, irregular and sesamoid. Long bones, especially the femur and tibia, can be subjected to most of the load during daily activities. Long bones grow primarily by elongation of the diaphysis, with an epiphysis at each end of the growing bone. The ends of epiphyses are covered with hyaline cartilage (“articular cartilage”). The longitudinal growth of long bones is a result of endochondral ossification at the epiphyseal plate. The long bone category type includes the femur, tibia, and fibula of the legs; the humerus, radius, and ulna of the arms; metacarpals and metatarsals of the hands and feet, the phalanges of the fingers and toes, and the clavicles or collar bones in humans or other patients. The outside of the long bone consists of a layer of connective tissue called the periosteum. Additionally, the outer shell of the long bone is compact bone, then a deeper layer of cancellous bone (spongy bone) which includes a medullary cavity that includes bone marrow. (Search “long bone” on Wikipedia.com May 14, 2021. CC-BY-SA 3.0 Modified. Accessed Jul. 26, 2021.)

“Talar dome” refers to part of a talus bone. Specifically, the talar dome refers to the superior convex surface and/or area of the talus. The talar dome may also be referred to as a trochlea of the talus. The talar dome is part of the talus body.

“Bone fragment” or “fragment” refers to a part of a bone that is normally part of another bone of a patient. A bone fragment may be separate from another bone of a patient due to a deformity or trauma. In one aspect, the bone the bone fragment is normally connected or joined with is referred to as a parent bone.

As used herein, “manufacturing tool” or “fabrication tool” refers to a manufacturing or fabrication process, tool, system, or apparatus which creates an object, device, apparatus, feature, or component using one or more source materials. A manufacturing tool or fabrication tool can use a variety of manufacturing processes, including but not limited to additive manufacturing, subtractive manufacturing, forging, casting, and the like. The manufacturing tool can use a variety of materials including polymers, thermoplastics, metals, biocompatible materials, biodegradable materials, ceramics, biochemicals, and the like. A manufacturing tool may be operated manually by an operator, automatically using a computer numerical controller (CNC), or a combination of these techniques.

As used herein, “osteotomy procedure” or “surgical osteotomy” refers to a surgical operation in which one or more bones are cut to shorten or lengthen them or to change their alignment. The procedure can include removing one or more portions of bone and/or adding one or more portions of bone or bone substitutes. (Search “osteotomy” on Wikipedia.com Feb. 3, 22, 2021. CC-BY-SA 3.0 Modified. Accessed Feb. 15, 2022.)

“Minimally invasive surgery” or “minimal invasive surgery” (MIS surgery) refers to one or more surgical techniques that limits the size of incisions needed for a surgical procedure, thereby reducing wound healing time, associated pain, and risk of infection. Surgery by definition is invasive and many operations requiring incisions of some size are referred to as open surgery. (Search “minimally invasive surgery” on Wikipedia.com Jan. 29, 2023. CC-BY-SA 3.0 Modified. Accessed Jan. 30, 2023.)

As used herein, “patient-specific osteotomy procedure” refers to an osteotomy procedure that has been adjusted, tailored, modified, or configured to specifically address the anatomy, physiology, condition, abnormalities, needs, or desires of a particular patient. In certain aspects, one patient-specific osteotomy procedure may be useable in connection with only one patient. In other aspects, one patient-specific osteotomy procedure may be useable with a number of patients having a particular class of characteristics. In certain aspects, a patient-specific osteotomy procedure may refer to a non-patient-specific osteotomy procedure that includes one or more patient-specific implants and/or instrumentation. In another aspects, a patient-specific osteotomy procedure may refer to a patient-specific osteotomy procedure that includes one or more patient-specific implants, patient-specific surgical steps, and/or patient-specific instrumentation.

“Register” or “Registration” refers to an act of aligning, mating, contacting, engaging, or coupling one or more parts and/or surfaces of one object in relation to one or more parts and/or surfaces of another object. Often, the one or more parts and/or surfaces one object include protrusions and/or depressions that are the inverse or mirror configuration of protrusions and/or depressions of one or more parts and/or surfaces of the other object.

“Remediation procedure” refers to any designed or performed for the purpose of remediating a condition of a patient and/or a condition of one or more parts of a body of a patient.

“Wedge osteotomy” refers to an osteotomy procedure in which one or more wedges are used as part of the procedure. Generally, wedge osteotomies can be of one of two types, open wedge and closing wedge. The type of osteotomy refers to how the procedure changes the relation between two parts of a bone involved in the osteotomy. In an open wedge osteotomy a wedge of bone or graft or other material is inserted in between two parts of a bone. Consequently, a wedge shape is “opened” in the bone. In a close wedge osteotomy or closing wedge osteotomy a wedge of bone is removed from a bone. Consequently, a wedge shape formed in the bone is “closed.”

As used herein, “anatomic data” refers to data identified, used, collected, gathered, and/or generated in connection with an anatomy of a human or animal. Examples of anatomic data may include location data for structures, both independent, and those connected to other structures within a coordinate system. Anatomic data may also include data that labels or identifies one or more anatomical structures. Anatomic data can include volumetric data, material composition data, and/or the like. Anatomic data can be generated based on medical imaging data or measurements using a variety of instruments including monitors and/or sensors. Anatomic data can be gathered, measured, or collected from anatomical models and/or can be used to generate, manipulate, or modify anatomical models.

A bone model or anatomic model of a patient's body or body part(s) may be generated by computing devices that analyze medical imaging images. Structures of a patient's body can be determined using a process called segmentation.

“Trajectory guide” or “trajectory indicator” or “targeting guide” refers to any structure, apparatus, surface, device, system, feature, or aspect configured to indicate, identify, guide, place, position, or otherwise assist in marking or deploying a fastener or other structure along a desired trajectory for one or more subsequent steps in a procedure.

“Patient-specific trajectory guide” refers to a trajectory guide that is patient-specific. In certain embodiments, a patient-specific trajectory guide is a trajectory guide that can only be used on or in connection with a surgical procedure for a specific/particular/unique patient. A patient-specific trajectory guide serves as a guide for a trajectory to address a condition for the specific patient.

“Patient-matched trajectory guide” refers to a trajectory guide that is patient-matched. In certain embodiments, a patient-matched trajectory guide is a trajectory guide that can be used on or in connection with a surgical procedure for a plurality of patient who share a common characteristic and/or attribute. A patient-matched trajectory guide serves as a guide for a trajectory to address a condition for the set of patients that satisfy one or more characteristics and/or attributes.

“Trajectory” refers to a path a body travels or a path configured for a body to travel through space. (Search “trajectory” on wordhippo.com. WordHippo, 2023. Web. Modified. Accessed 13 Jun. 2023.)

As used herein, “side” refers to a structure or part of a structure including, but not limited to one of a longer bounding surfaces or lines of an object especially contrasted with the ends, a line or surface forming a border or face of an object, either surface of a thin object, a bounding line or structure of a geometric figure or shape, and the like. (search “side” on Merriam-Webster.com. Merriam-Webster, 2021. Web. 3 Aug. 2021. Modified.) A side can also refer to a geometric edge of a polygon (two-dimensional shape) and/or a face or surface of a polyhedron (three-dimensional shape). (Search “side” on Wikipedia.com Jul. 21, 2021. CC-BY-SA 3.0 Modified. Accessed Aug. 3, 2021.) Side can also refer to a location on a structure. For example, a side can be a location on a structure at, or near, a furthest position away from a central axis of the structure. As used herein, the term “side” can include one or more modifiers that define and/or orient and/or distinguish the side of an object from others based on based on where and/or how the object is deployed within or in relation to a second object. For example, in the context of an implant for a patient, sides of the implant may be labeled based on where the sides are relative to the patient when the implant is deployed. As one example, an “anterior side” of an implant, instrument, anatomical structure, or other structure refers to a side that is anterior to other sides of the structure in relation to a patient when the structure is deployed in the patient. As another example, in the context of an instrument used with a patient, sides of the instrument may be labeled based on where the sides are when the instrument is being used for its purpose. As one example, a “front side” of an instrument refers to a side that is facing a user of the instrument when the instrument is in use.

As used herein, a “guard” refers to an apparatus, instrument, structure, member, device, component, system, or assembly structured, organized, configured, designed, arranged, or engineered to prevent, limit, impede, stop, or restrict motion, action, or movement and/or operation of the another object, member, structure, component, part, apparatus, system, or assembly beyond a certain parameter such as a boundary. Said another way, a “guard” refers to an apparatus, instrument, structure, member, device, component, system, or assembly structured, organized, configured, designed, arranged, or engineered to retain, maintain, hold, keep, or restrict motion, action, or movement and/or operation of the another object, member, structure, component, part, apparatus, system, or assembly within or at one or more parameters such as a boundary.

As used herein, “artificial intelligence” refers to intelligence demonstrated by machines, unlike the natural intelligence displayed by humans and animals, which involves consciousness and emotionality. The distinction between artificial intelligence and natural intelligence categories is often revealed by the acronym chosen. ‘Strong’ AI is usually labelled as artificial general intelligence (AGI) while attempts to emulate ‘natural’ intelligence have been called artificial biological intelligence (ABI). Leading AI textbooks define the field as the study of “intelligent agents”: any device that perceives its environment and takes actions that maximize its chance of achieving its goals. The term “artificial intelligence” can also be used to describe machines that mimic “cognitive” functions that humans associate with the human mind, such as “learning” and “problem solving”. (Search “artificial intelligence” on Wikipedia.com Jun. 25, 2021. CC-BY-SA 3.0 Modified. Accessed Jun. 25, 2021.)

As used herein, “segmentation” or “image segmentation” refers the process of partitioning an image into different meaningful segments. These segments may correspond to different tissue classes, organs, pathologies, bones, or other biologically relevant structures. Medical image segmentation accommodates imaging ambiguities such as by low contrast, noise, and other imaging ambiguities.

Certain computer vision techniques can be used or adapted for image segmentation. For example, the techniques and or algorithms for segmentation may include, but are not limited to: Atlas-Based Segmentation: For many applications, a clinical expert can manually label several images; segmenting unseen images is a matter of extrapolating from these manually labeled training images. Methods of this style are typically referred to as atlas-based segmentation methods. Parametric atlas methods typically combine these training images into a single atlas image, while nonparametric atlas methods typically use all of the training images separately. Atlas-based methods usually require the use of image registration in order to align the atlas image or images to a new, unseen image.

Image registration is a process of correctly aligning images; Shape-Based Segmentation: Many methods parametrize a template shape for a given structure, often relying on control points along the boundary. The entire shape is then deformed to match a new image. Two of the most common shape-based techniques are Active Shape Models and Active Appearance Models; Image-Based Segmentation: Some methods initiate a template and refine its shape according to the image data while minimizing integral error measures, like the Active contour model and its variations; Interactive Segmentation: Interactive methods are useful when clinicians can provide some information, such as a seed region or rough outline of the region to segment. An algorithm can then iteratively refine such a segmentation, with or without guidance from the clinician. Manual segmentation, using tools such as a paint brush to explicitly define the tissue class of each pixel, remains the gold standard for many imaging applications. Recently, principles from feedback control theory have been incorporated into segmentation, which give the user much greater flexibility and allow for the automatic correction of errors; Subjective surface Segmentation: This method is based on the idea of evolution of segmentation function which is governed by an advection-diffusion model. To segment an object, a segmentation seed is needed (that is the starting point that determines the approximate position of the object in the image). Consequently, an initial segmentation function is constructed. With the subjective surface method, the position of the seed is the main factor determining the form of this segmentation function; and Hybrid segmentation which is based on combination of methods. (Search “medical image computing” on Wikipedia.com Jun. 24, 2021. CC-BY-SA 3.0 Modified. Accessed Jun. 24, 2021.)

As used herein, “medical imaging” refers to a technique and process of imaging the interior of a body for clinical analysis and medical intervention, as well as visual representation of the function of some organs or tissues (physiology). Medical imaging seeks to reveal internal structures hidden by the skin and bones, as well as to diagnose and treat disease. Medical imaging may be used to establish a database of normal anatomy and physiology to make possible identification of abnormalities. Medical imaging in its widest sense, is part of biological imaging and incorporates radiology, which uses the imaging technologies of X-ray radiography, magnetic resonance imaging, ultrasound, endoscopy, elastography, tactile imaging, thermography, medical photography, nuclear medicine functional imaging techniques as positron emission tomography (PET) and single-photon emission computed tomography (SPECT). Another form of X-ray radiography includes computerized tomography (CT) scans in which a computer controls the position of the X-ray sources and detectors. Magnetic Resonance Imaging (MRI) is another medical imaging technology. Measurement and recording techniques that are not primarily designed to produce images, such as electroencephalography (EEG), magnetoencephalography (MEG), electrocardiography (ECG), and others, represent other technologies that produce data susceptible to representation as a parameter graph vs. time or maps that contain data about the measurement locations. In certain embodiments bone imaging includes devices that scan and gather bone density anatomic data. These technologies may be considered forms of medical imaging in certain disciplines. (Search “medical imaging” on Wikipedia.com Jun. 16, 2021. CC-BY-SA 3.0 Modified. Accessed Jun. 23, 2021.) Data, including images, text, and other data associated with medical imaging is referred to as patient imaging data. As used herein, “patient imaging data” refers to data identified, used, collected, gathered, and/or generated in connection with medical imaging and/or medical imaging data. Patient imaging data can be shared between users, systems, patients, and professionals using a common data format referred to as Digital Imaging and Communications in Medicine (DICOM) data. DICOM data is a standard format for storing, viewing, retrieving, and sharing medical images.

As used herein, “medical image computing” or “medical image processing” refers to systems, software, hardware, components, and/or apparatus that involve and combine the fields of computer science, information engineering, electrical engineering, physics, mathematics and medicine. Medical image computing develops computational and mathematical methods for working with medical images and their use for biomedical research and clinical care. One goal for medical image computing is to extract clinically relevant information or knowledge from medical images. While closely related to the field of medical imaging, medical image computing focuses on the computational analysis of the images, not their acquisition. The methods can be grouped into several broad categories: image segmentation, image registration, image-based physiological modeling, and others. (Search “medical image computing” on Wikipedia.com Jun. 24, 2021. CC-BY-SA 3.0 Modified. Accessed Jun. 24, 2021.) Medical image computing may include one or more processors or controllers on one or more computing devices. Such processors or controllers may be referred to herein as medical image processors. Medical imaging and medical image computing together can provide systems and methods to image, quantify and fuse both structural and functional information about a patient in vivo. These two technologies include the transformation of computational models to represent specific subjects/patients, thus paving the way for personalized computational models. Individualization of generic computational models through imaging can be realized in three complementary directions: definition of the subject-specific computational domain (anatomy) and related subdomains (tissue types); definition of boundary and initial conditions from (dynamic and/or functional) imaging; and characterization of structural and functional tissue properties. Medical imaging and medical image computing enable in the translation of models to the clinical setting with both diagnostic and therapeutic applications. (Id.) In certain embodiments, medical image computing can be used to generate a bone model, a patient-specific model, and/or a patent specific instrument from medical imaging and/or medical imaging data.

As used herein, “model” refers to an informative representation of an object, person or system. Representational models can be broadly divided into the concrete (e.g. physical form) and the abstract (e.g. behavioral patterns, especially as expressed in mathematical form). In abstract form, certain models may be based on data used in a computer system or software program to represent the model. Such models can be referred to as computer models. Computer models can be used to display the model, modify the model, print the model (either on a 2D medium or using a 3D printer or additive manufacturing technology). Computer models can also be used in environments with models of other objects, people, or systems. Computer models can also be used to generate simulations, display in virtual environment systems, display in augmented reality systems, or the like. Computer models can be used in Computer Aided Design (CAD) and/or Computer Aided Manufacturing (CAM) systems. Certain models may be identified with an adjective that identifies the object, person, or system the model represents. For example, a “bone” model is a model of a bone, and a “heart” model is a model of a heart. (Search “model” on Wikipedia.com Jun. 13, 2021. CC-BY-SA 3.0 Modified. Accessed Jun. 23, 2021.) As used herein, “additive manufacturing” refers to a manufacturing process in which materials are joined together in a process that repeatedly builds one layer on top of another to generate a three-dimensional structure or object. Additive manufacturing may also be referred to using different terms including additive processes, additive fabrication, additive techniques, additive layer manufacturing, layer manufacturing, freeform fabrication, ASTM F2792 (American Society for Testing and Materials), and 3D printing. Additive manufacturing can build the three-dimensional structure or object using computer-controlled equipment that applies successive layers of the material(s) based on a three-dimensional model that may be defined using Computer Aided Design (CAD) software. Additive manufacturing can use a variety of materials including polymers, thermoplastics, metals, ceramics, biochemicals, and the like. Additive manufacturing may provide unique benefits, as an implant together with the pores and/or lattices can be directly manufactured (without the need to generate molds, tool paths, perform any milling, and/or other manufacturing steps).

“Repository” refers to any data source or dataset that includes data or content. In one embodiment, a repository resides on a computing device. In another embodiment, a repository resides on a remote computing or remote storage device. A repository may comprise a file, a folder, a directory, a set of files, a set of folders, a set of directories, a database, an application, a software application, content of a text, content of an email, content of a calendar entry, and the like. A repository, in one embodiment, comprises unstructured data. A repository, in one embodiment, comprises structured data such as a table, an array, a queue, a look up table, a hash table, a heap, a stack, or the like. A repository may store data in any format including binary, text, encrypted, unencrypted, a proprietary format, or the like.

As used herein, “registration” or “ image registration” refers to a method, process, module, component, apparatus, and/or system that seeks to achieve precision in the alignment of two images. As used here, “image” may refer to either or both an image of a structure or object and another image or a model (e.g., a computer based model or a physical model, in either two dimensions or three dimensions). In the simplest case of image registration, two images are aligned. One image may serve as the target image and the other as a source image; the source image is transformed, positioned, realigned, and/or modified to match the target image. An optimization procedure may be applied that updates the transformation of the source image based on a similarity value that evaluates the current quality of the alignment. An iterative procedure of optimization may be repeated until a (local) optimum is found. An example is the registration of CT and PET images to combine structural and metabolic information. Image registration can be used in a variety of medical applications: Studying temporal changes; Longitudinal studies may acquire images over several months or years to study long-term processes, such as disease progression. Time series correspond to images acquired within the same session (seconds or minutes). Time series images can be used to study cognitive processes, heart deformations and respiration; Combining complementary information from different imaging modalities. One example may be the fusion of anatomical and functional information.

Since the size and shape of structures vary across modalities, evaluating the alignment quality can be more challenging. Thus, similarity measures such as mutual information may be used; Characterizing a population of subjects. In contrast to intra-subject registration, a one-to-one mapping may not exist between subjects, depending on the structural variability of the organ of interest. Inter-subject registration may be used for atlas construction in computational anatomy. Here, the objective may be to statistically model the anatomy of organs across subjects; Computer-assisted surgery: in computer-assisted surgery pre-operative images such as CT or MRI may be registered to intra-operative images or tracking systems to facilitate image guidance or navigation. There may be several considerations made when performing image registration: The transformation model. Common choices are rigid, affine, and deformable transformation models. B-spline and thin plate spline models are commonly used for parameterized transformation fields. Non-parametric or dense deformation fields carry a displacement vector at every grid location; this may use additional regularization constraints. A specific class of deformation fields are diffeomorphisms, which are invertible transformations with a smooth inverse; The similarity metric. A distance or similarity function is used to quantify the registration quality. This similarity can be calculated either on the original images or on features extracted from the images. Common similarity measures are sum of squared distances (SSD), correlation coefficient, and mutual information. The choice of similarity measure depends on whether the images are from the same modality; the acquisition noise can also play a role in this decision. For example, SSD may be the optimal similarity measure for images of the same modality with Gaussian noise. However, the image statistics in ultrasound may be significantly different from Gaussian noise, leading to the introduction of ultrasound specific similarity measures.

Multi-modal registration may use a more sophisticated similarity measure; alternatively, a different image representation can be used, such as structural representations or registering adjacent anatomy; The optimization procedure. Either continuous or discrete optimization is performed. For continuous optimization, gradient-based optimization techniques are applied to improve the convergence speed.(Search “medical image computing” on Wikipedia.com Jun. 24, 2021. CC-BY-SA 3.0 Modified. Accessed Jun. 25, 2021.)

As used herein, a “resection” refers to a method, procedure, or step that removes tissue from another anatomical structure or body. A resection is typically performed by a surgeon on a part of a body of a patient. (Search “surgery” on Wikipedia.com May 26, 2021. CC-BY-SA 3.0 Modified. Accessed May 26, 2021.) Resection may be used as a noun or a verb. In the verb form, the term is “resect” and refers to an act of performing, or doing, a resection. Past tense of the verb resect is resected.

“Bone condition” refers to any of a variety of conditions of bones of a patient. Generally, a bone condition refers to an orientation, position, and/or alignment of one or more bones of the patient relative to other anatomical structures of the body of the patient. Bone conditions may be caused by or result from deformities, misalignment, malrotation, fractures, joint failure, and/or the like. A bone condition includes, but is not limited to, any angular deformities of one or more bone segments in either the lower or upper extremities (for example, tibial deformities, calcaneal deformities, femoral deformities, and radial deformities). Alternatively, or in addition, “bone condition” can refer to the structural makeup and configuration of one or more bones of a patient. Thus bone condition may refer to a state or condition of regions, a thickness of a cortex, bone density, a thickness and/or porosity of internal regions (e.g. whether it is calcaneus or solid) of the bone or parts of the bone such as a head, a base, a shaft, a protuberance, a process, a lamina, a foramen, and the like of a bone, along the metaphyseal region, epiphysis region, and/or a diaphyseal region. “Malrotation” refers to a condition in which a part, typically a part of a patient's body has rotated from a normal position to an unnormal or uncommon position.

As used herein, a “guide” refers to a part, component, member, or structure designed, adapted, configured, or engineered to guide or direct one or more other parts, components, or structures. A guide may be part of, integrated with, connected to, attachable to, or coupled to, another structure, device, or instrument. In one embodiment, a guide may include a modifier that identifies a particular function, location, orientation, operation, type, and/or a particular structure of the guide. Examples of such modifiers applied to a guide, include, but are not limited to, “pin guide” that guides or directs one or more pins, a “cutting guide” that guides or directs the making or one or more cuts, a placement, deployment, or insertion guide that guides or directs the placement, positioning, orientation, deployment, installation, or insertion of a fastener and/or implant, a “cross fixation guide” that guides deployment of a fastener or fixation member, an “alignment guide” that guides the alignment of two or more objects or structures, a “resection guide” that serves to guide resection of soft or hard tissue, such as in an osteotomy, a “reduction guide” can serve to guide reduction of one or more bone segments or fragments, an “placement guide” that serves to identify how an object can be placed in relation to another object or structure, a “fixation guide” that guides deployment of fasteners or other fixation structures, and the like. Furthermore, guides may include modifiers applied due to the procedure or location within a patient for which the guide is to be used. For example, where a guide is used at a joint, the guide may be referred to herein as an “arthrodesis guide”.

As used herein, “feature” refers to a distinctive attribute or aspect of something. (Search “feature” on google.com. Oxford Languages, 2021. Web. 20 Apr. 2021.) A feature may include one or more apparatuses, structures, objects, systems, sub-systems, devices, or the like. A feature may include a modifier that identifies a particular function or operation and/or a particular structure relating to the feature. Examples of such modifiers applied to a feature, include, but are not limited to, “attachment feature,” “securing feature,” “placement feature,” “protruding feature,” “engagement feature,” “disengagement feature,” “resection feature”, “guide feature”, and the like.

“Cortical bone” refers to a type of bone tissue. Cortical bone is a type of bone tissue typically found between an external surface of a bone and an interior area of the bone. Cortical bone is more dense and typically stronger structurally than other types of bone tissue. “Cortical surface” refers to a surface of cortical bone.

“Transosseous placement feature” refers to a placement feature that extends through one or more bones and that enables, or facilitates, placement of another device, apparatus, or instrument.

“Patient specific feature” refers to a feature, function, structure, device, guide, tool, instrument, apparatus, member, component, system, assembly, module, or subsystem that is adjusted, tailored, modified, organized, configured, designed, arranged, engineered, and/or fabricated to specifically address the anatomy, physiology, condition, abnormalities, needs, or desires of a particular patient or surgeon serving the particular patient. In one aspect, a patient specific feature is unique to a single patient and may include features unique to the patient such as a number of cut channels, a number of bone attachment features, a number of bone engagement surfaces, a number of resection features, a depth of one or more cutting channels, an angle for one or more resection channels, a surface contour, component position, component orientation, and/or other features. “Medial resection guide” refers to a resection guide designed, engineered, fabricated, or intended for use with, one, in, or about a medial part, section, surface, portion, or aspect of an anatomical structure such as a bone, digit, limb, or other anatomical structure for one or more steps of a resection procedure. “Lateral resection guide” refers to a resection guide designed, engineered, fabricated, or intended for use with, one, in, or about a lateral part, section, surface, portion, or aspect of an anatomical structure such as a bone, digit, limb, or other anatomical structure for one or more steps of a resection procedure.

“Bone fragment” refers to a part of a bone that is normally part of another bone of a patient. A bone fragment may be separate from another bone of a patient due to a deformity or trauma. In one aspect, the bone the bone fragment is normally connected or joined with is referred to as a parent bone.

“Cut surface” refers to a surface of an object that is created or formed by the removal of one or more parts of the object that includes the original surface. Cut surfaces can be created using a variety of methods, tools, or apparatuses and may be formed using a variety of removal actions, including, but not limited to, fenestrating, drilling, abrading, cutting, sawing, chiseling, digging, scrapping, and the like. Tools and/or methods used for forming a cut surface can include manual, mechanical, motorized, hydraulic, automated, robotic, and the like. In certain embodiments, the cut surface(s) are planar.

“Orientation” refers to a direction, angle, position, condition, state, or configuration of a first object, component, part, apparatus, system, or assembly relative to another object, component, part, apparatus, system, assembly, reference point, reference axis, or reference plane.

“Longitudinal axis” refers to an axis of a structure, device, object, apparatus, or part thereof that extends from one end of a longest dimension to an opposite end. Typically, a longitudinal axis passes through a center of the structure, device, object, apparatus, or part thereof along the longitudinal axis. The center point used for the longitudinal axis may be a geometric center point and/or a mass center point.

Those of skill in the art will appreciate that a resection feature may take a variety of forms and may include a single feature or one or more features that together form the resection feature. In certain embodiments, the resection feature may take the form of one or more slots. Alternatively, or in addition, a resection feature may be referenced using other names including, but not limited to, channel, cut channels, and the like.

“Cutting tool” refers to any tool that can be used to cut or resect another object. In particular, a cutting tool can refer to a manual or power tool for cutting or resecting tissue of a patient. Examples of cutting tools include, but are not limited to, a burr, an oscillating saw, a reciprocating saw, a grater saw, a drill, a mill, a side-cutting burr, or the like.

The present disclosure discloses surgical systems and methods by which a bone condition, that can include a deformity, may be corrected or otherwise addressed. Known methods of addressing bone conditions are often limited to a finite range of discretely sized instruments. A patient with an unusual condition, or anatomy that falls between instrument sizes, may not be readily treated with such systems.

Furthermore, patient-specific guides may be used for various other procedures on the foot, or on other bones of the musculoskeletal system. For example, patient-specific guides may be used for various surgical procedure.

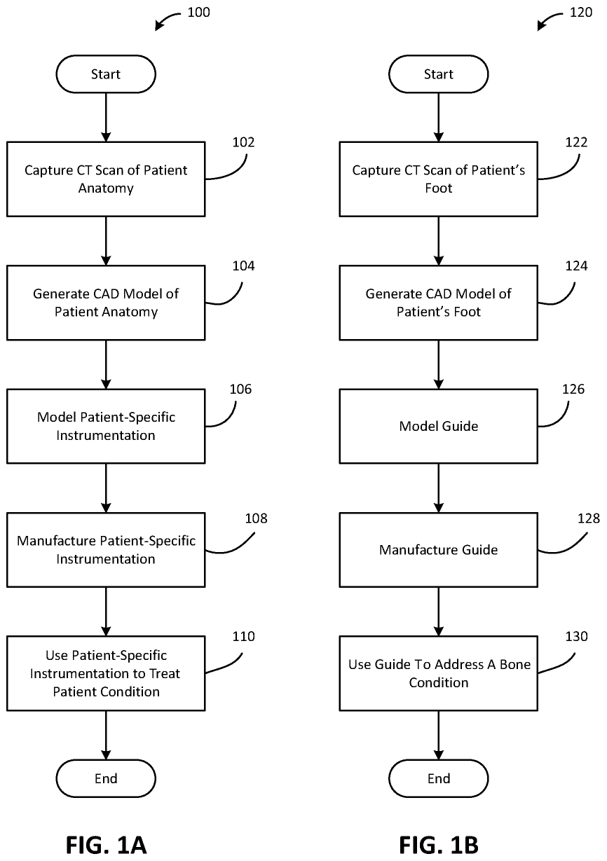

FIG. 1A is a flowchart diagram depicting a method 100 for correcting a bone condition, according to one embodiment. The method 100 may be used for any of a wide variety of bone conditions, including but not limited to deformities, fractures, joint failure, and/or the like. Further, the method 100 may provide correction with a wide variety of treatments, including but not limited to arthroplasty, arthrodesis, fracture repair, and/or the like.

As shown, the method 100 may begin with a step 102 in which a CT scan (or another three-dimensional image, also referred to as medical imaging) of the patient's anatomy is obtained. The step 102 may entail capturing a scan of only the particular bone(s) to be treated, or may entail capture of additional anatomic information, such as the surrounding tissues. Additionally or alternatively, the step 102 may entail receiving a previously captured image, for example, at a design and/or fabrication facility. Performance of the step 102 may result in possession of a three-dimensional model of the patient's anatomy, or three-dimensional surface points that can be used to construct such a three-dimensional model.

After the step 102 has been carried out, the method 100 may proceed to a step 104 in which a CAD model of the patient's anatomy (including one or more bones) is generated. The CAD model may be one example of a bone model. The CAD model may be of any known format, including but not limited to SolidWorks, Catia, AutoCAD, or DXF. In some embodiments, customized software may be used to generate the CAD model from the CT scan. The CAD model may only include the bone(s) to be treated and/or may include surrounding tissues. In alternative embodiments, the step 104 may be omitted, as the CT scan may capture data that can directly be used in future steps without the need for conversion.

In one embodiment, the CAD model generated and/or patient-specific instrumentation, implants, and/or plan for conducting an operative procedure, may be enhanced by the use of advanced computer analysis system, machine learning, and/or automated/artificial intelligence. For example, these technologies may be used to revise a set of steps for a procedure such that a more desirable outcome is achieved.

In a step 106, the CAD model and/or CT scan data may be used to model patient-specific instrumentation that can be used to correct the condition, as it exists in the patient's anatomy. In some embodiments, any known CAD program may be used to view and/or manipulate the CAD model and/or CT scan, and generate one or more instruments that are matched specifically to the size and/or shape of the patient's bone(s). In some embodiments, such instrumentation may include a targeting guide, trajectory guide, drill guide, cutting guide, tendon trajectory guide or similar guide that can be attached to one or more bones, with one or more features that facilitate work on the one or more bones pursuant to a procedure such as arthroplasty or arthrodesis. In some embodiments, performance of the step 106 may include modelling an instrument with a bone engagement surface that is shaped to match the contour of a surface of the bone, such that the bone engagement surface can lie directly on the corresponding contour.

In a step 108, the model(s) may be used to manufacture patient-specific instrumentation and/or implants. This may be done via any known manufacturing method, including casting, forging, milling, additive manufacturing, and/or the like. Additive manufacturing may provide unique benefits, as the model may be directly used to manufacture the instrumentation and/or implants (without the need to generate molds, tool paths, and/or the like beforehand). Such instrumentation may optionally include a targeting guide, trajectory guide, drill guide, cutting guide, or tendon trajectory guide with the bone engagement surface and one or more features as described herein.

In addition to, or in the alternative to the step 108, the model(s) may be used to select from available sizes of implants and/or instruments and advise the surgeon accordingly. For example, where a range of guides are available for a given procedure, analysis of the CAD data may facilitate pre-operative selection of the optimal guide and/or optimal placement of the guide on the bone. Similarly, if a range of implants may be used for a given procedure, analysis of the CAD data may facilitate pre-operative selection of the optimal implant(s). More particularly, properly-sized spacers, screws, bone plates, and/or other hardware may be pre-operatively selected.

Thus, the result of the step 108 may provision, to the surgeon, of one or more of the following: (1) one or more patient-specific instruments; (2) one or more patient-specific implants; (3) an instrument, selected from one or more available instrument sizes and/or configurations; (4) an implant, selected from one or more available implant sizes and/or configurations; (5) instructions for which instrument(s) to select from available instrument sizes and/or configurations; (6) instructions for which implant(s) to select from available implant sizes and/or configurations; (7) instructions for proper positioning or anchorage of one or more instruments to be used in the procedure; and (8) instructions for proper positioning or anchorage of one or more implants to be used in the procedure. These items may be provided to the surgeon directly, or to a medical device company or representative, for subsequent delivery to the surgeon.

In a step 110, the manufactured instrumentation may be used in surgery to facilitate treatment of the condition. In some embodiments, this may entail placing the modelled bone engagement surface against the corresponding contour of the bone used to obtain its shape, and then using the resection feature(s) to guide resection of one or more bones. Then the bone(s) may be further treated, for example, by attaching one or more joint replacement implants (in the case of joint arthroplasty), or by attaching bone segments together (in the case of arthrodesis or fracture repair). Prior to completion of the step 110, the instrumentation may be removed from the patient, and the surgical wound may be closed.

As mentioned previously, the method 100 may be used to correct a wide variety of bone conditions. One example of the method 100 will be shown and described in connection with FIG. 1B, for correction of a bunion deformity of the foot.

In certain embodiments, one or more of a method, apparatus, and/or system of the disclosed solution can be used for training a surgeon to perform a patient-specific procedure or technique. In one embodiment, the CAD model generated and/or patient-specific instrumentation, implants, and/or plan for conducting an operative procedure can be used to train a surgeon to perform a patient-specific procedure or technique.

In one example embodiment, a surgeon may submit a CT scan of a patient's foot to an apparatus or system that implements the disclosed solution. Next, a manual or automated process may be used to generate a CAD model and for making the measurements and correction desired for the patient. In the automated process, advanced computer analysis system, machine learning and automated/artificial intelligence may be used to generate a CAD model and/or one or more patient-specific instruments and/or operation plans. For example, a patient-specific guide may be fabricated that is registered to the patient's anatomy using a computer-aided machine (CAM) tool. In addition, a CAM tool may be used to fabricate a 3D structure representative of the patient's anatomy, referred to herein as a patient-specific synthetic cadaver. (e.g. one or more bones of a patient's foot). Next, the patient-specific guide and the patient-specific synthetic cadaver can be provided to a surgeon who can then rehearse an operation procedure in part or in full before going into an operating room with the patient.

In certain embodiments, the patient-specific guide or instrument can be used to preposition and/or facilitate pre-drilling holes for a plate system for fixation purposes. Such plate systems may be optimally placed, per a CT scan, after a correction procedure for optimal fixation outcome. In another embodiment, the CAD model and/or automated process such as advanced computer analysis, machine learning and automated/artificial intelligence may be used to measure a depth of the a through a patient-specific resection guide for use with robotics apparatus and/or systems which would control the depth of each cut within the guide to protect vital structures below or adjacent to a bone being cut. In another embodiment, the CAD model and/or automated process such as advanced computer analysis, machine learning and automated/artificial intelligence may be used to define desired fastener (e.g. bone screw) length and/or trajectories through a patient-specific guide and/or implant. The details for such lengths, trajectories, and components can be detailed in a report provided to the surgeon preparing to perform a procedure.

FIG. 1B is a flowchart diagram depicting a method 120 for correcting or remediating a bone condition, according to one embodiment. The method 120 may be used to prepare for an orthopedic procedure which corrects or remediates a bone, muscle, and/or tendon condition of a patient.

As shown, the method 120 may begin with a step 122 in which a CT scan (or another three-dimensional image) of the patient's foot is obtained. The step 122 may entail capturing a scan of only the first cuneiform and first metatarsal, or may entail capture of additional anatomic information, such as the entire foot. Additionally or alternatively, the step 122 may entail receipt of previously captured image data. Capture of the entire foot in the step 122 may facilitate proper alignment of the first metatarsal with the rest of the foot (for example, with the second metatarsal). Performance of the step 122 may result in generation of a three-dimensional model of the patient's foot, or three-dimensional surface points that can be used to construct such a three-dimensional model.

After the step 122 has been carried out, the method 120 may proceed to a step 124 in which a CAD model of the relevant portion of the patient's anatomy is generated. The CAD model may optionally include the bones of the entire foot, like the CT scan obtained in the step 122. In alternative embodiments, the step 124 may be omitted in favor of direct utilization of the CT scan data, as described in connection with the step 104.

In a step 126, the CAD model and/or CT scan data may be used to model patient-specific instrumentation that can be used to correct or remediate a bone condition. Such instrumentation may include a guide. In one example, the guide can seat or abut or contact a surface of a bone and including an opening that guides a trajectory for a fastener for a procedure. In some embodiments, performance of the step 126 may include modelling the guide with a bone engagement surface that is shaped to match contours of the surfaces of the bone, such that the bone engagement surface can lie directly on the corresponding contours of the bone.

In a step 128, the model(s) may be used to manufacture patient-specific instrumentation and/or instruments. This may include manufacturing the guide with the bone engagement surface and/or other features as described above. As in the step 108, the step 128 may additionally or alternatively involve provision of one or more instruments and/or implants from among a plurality of predetermined configurations or sizes. Further, the step 128 may additionally, or alternatively, involve provision of instructions for placement and/or anchorage of one or more instruments and/or instruments to carry out the procedure.

In a step 130, the manufactured guide may be used in surgery to facilitate treatment of the condition. Specifically, the bone engagement surface of the guide may be placed against the corresponding contours of the bone. The guide may include an opening and/or trajectory guide to guide insertion of a trajectory guide such as a temporary fastener such as a K-wire. The guide may then be removed, and the remaining steps of a surgical procedure performed.

The method 100 and the method 120 are merely exemplary. Those of skill in the art will recognize that various steps of the method 100 and the method 120 may be reordered, omitted, and/or supplemented with additional steps not specifically shown or described herein.

As mentioned previously, the method 120 is one species of the method 100; the present disclosure encompasses many different procedures, performed with respect to many different bones and/or joints of the body. Exemplary steps and instrumentation for the method 120 will further be shown and described in connection with the present disclosure. Those of skill in the art will recognize that the method 120 may be used in connection with different instruments; likewise, the instruments of the present disclosure may be used in connection with methods different from the method 100 and the method 120.

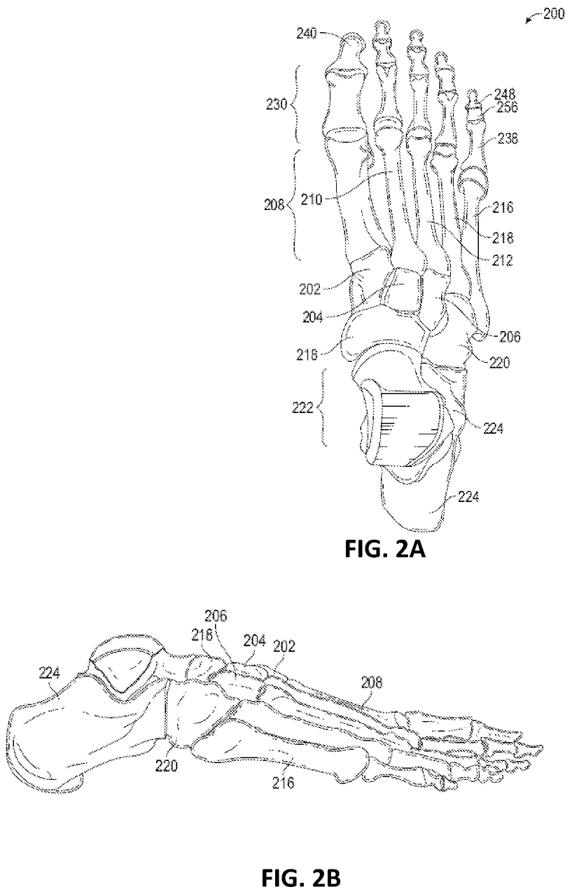

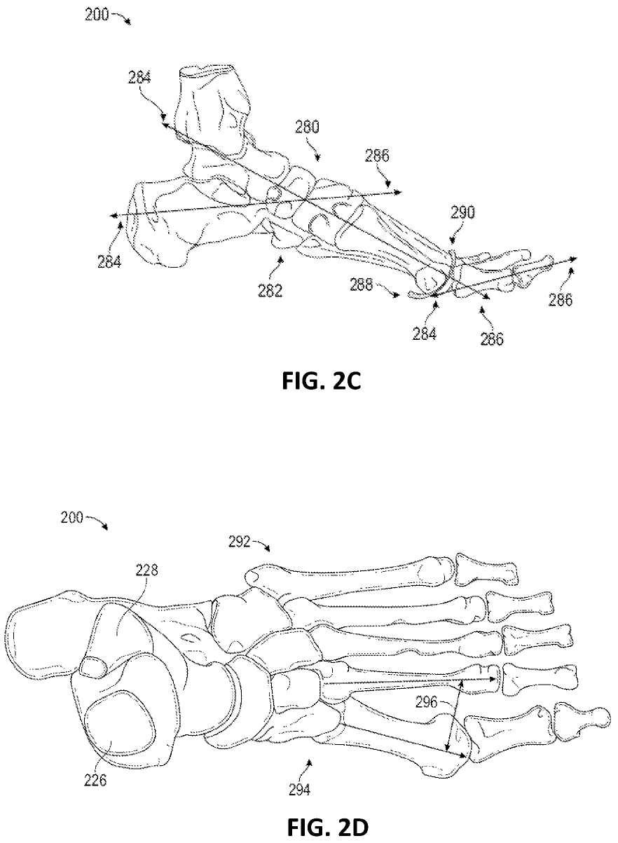

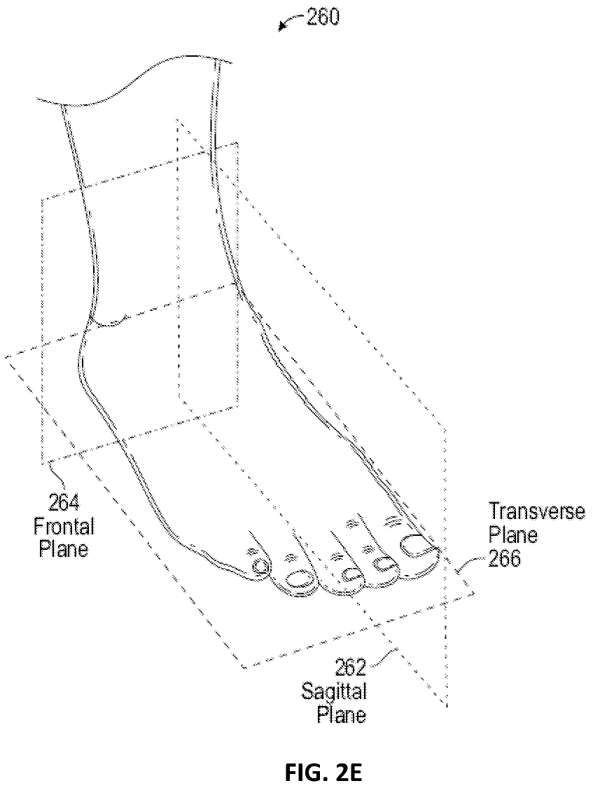

FIG. 2A is a perspective dorsal view of a foot 200. The foot 200 may have a medial cuneiform 202, an intermediate cuneiform 204, lateral cuneiform 206, a first metatarsal 208, a second metatarsal 210, third metatarsal 212, fourth metatarsal 214, fifth metatarsal 216, navicular 218, cuboid 220, talus 222, and calcaneus 224, among others. The medial cuneiform 202 and the intermediate cuneiform 204 may be joined together at a first metatarsocuneiform joint, and the first metatarsal 208 and the second metatarsal 210 may be joined together at a second metatarsocuneiform joint. The foot 200 includes a set of proximal phalanges numbered first through fifth (230, 232, 234, 236, 238) and a set of distal phalanges numbered first through fifth (240, 242, 244, 246, 248) and a set of middle phalanges numbered second through fifth (250, 252, 254, 256).

FIG. 2B is a perspective lateral view of a foot 200, with bones of the foot labeled.

FIG. 2C is a perspective medial view of a foot illustrating a dorsal side 280 and a plantar side 282. The foot 200, as illustrated, may have a tibia 226 and a fibula 228, among others. Dorsal refers to the top of the foot. Plantar refers to the bottom of the foot. Proximal 284 is defined as “closer to the primary attachment point”. Distal 286 is defined as “further away from the attachment point”. Plantarflex or plantarflexion 288 means movement toward the plantar side 282 of a foot or hand, toward the sole or palm. Dorsiflex or dorsiflexion 290 means movement toward the dorsal side 280 of a foot or hand, toward the top. FIG. 2D is a perspective dorsal view of the foot 200. A transverse plane is the plane that shows the top of the foot. A lateral side 292 means a side furthest away from the midline of a body, or away from a plane of bilateral symmetry of the body. A medial side 294 means a side closest to the midline of a body, or toward a plane of bilateral symmetry of the body. For a Lapidus procedure, the intermetatarsal (IM) angle 296 is the angle to be corrected to remove the hallux valgus (bunion) deformity.

FIG. 2E is a view of a foot illustrating common planes 260 of reference for a human foot. FIG. 2E illustrates a sagittal plane 262 that divides the foot into a right section and a left section half. The sagittal plane 262 is perpendicular to frontal or coronal plane 264 and the transverse plane 266. In the foot, the frontal plane 264 generally runs vertically through the ankle and the transverse plane 266 generally runs horizontally through the midfoot and toes of the foot.

Every patient and/or condition is different; accordingly, the degree of angular adjustment needed in each direction may be different for every patient. Use of a patient-specific guide may help the surgeon obtain an optimal realignment, target, or position a bone tunnel, position one or more resections and/or fasteners and the like. Thus, providing patient-specific guides, jigs, and/or instrumentation may provide unique benefits.

The present patient-specific instrumentation may be used to correct a wide variety of bone conditions. Such conditions include, but are not limited to, any angular deformities from within one bone segment in either the lower or upper extremities (for example, tibial deformities, calcaneal deformities, femoral deformities, and radial deformities). The present disclosure may also be used to treat an interface between two bone segments (for example, the ankle joint, metatarsal cuneiform joint, lisfranc's joint, complex Charcot deformity, wrist joint, knee joint, etc.). As one example, an angular deformity or segmental malalignment in the forefoot may be treated, such as is found at the metatarsal cuneiform level, the midfoot level such as the navicular cuneiform junction, hindfoot at the calcaneal cuboid or subtalar joint or at the ankle between the tibia and talar junction. Additionally, patient-specific instruments could be used in the proximal leg between two bone segments or in the upper extremity such as found at the wrist or metacarpal levels.

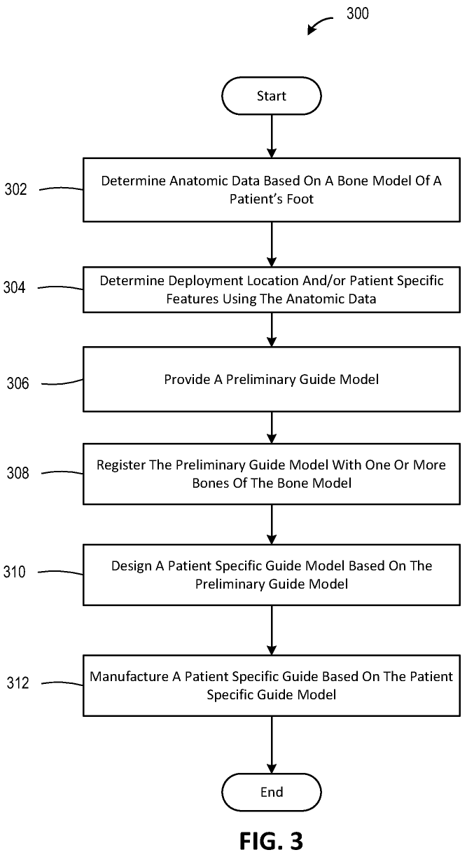

FIG. 3 illustrates a flowchart diagram depicting a method 300 for generating one or more patient-specific instruments configured to correct or address a bone or foot condition, according to one embodiment. Prior to steps of the method 300, a bone model (also referred to as CAD model above) is generated. The bone model may be generated using medical imaging of a patient's foot and may also be referred to as an anatomic model. The medical imaging image(s) may be used by computing devices to generate patient imaging data. The patient imaging data may be used to measure and account for orientation of one or more structures of a patient's anatomy. In certain embodiments, the patient imaging data may serve, or be a part of, anatomic data for a patient.

In one embodiment, the method 300 begins after a bone model of a patient's body or body part(s) is generated. In a first step 302, the method 300 may review the bone model and data associated with the bone model to determine anatomic data of a patient's foot.

After step 302, the method 300 may determine 304 a recommended location and/or a trajectory angle and/or patient-specific features for a procedure using the anatomic data. “Recommended location” refers to a location for deployment of guide or instrument on, in, between, or within one or more body parts (e.g., bones) of a patient. “Trajectory angle” refers to a recommended angle for deployment of an instrument, graft, body part, or resection feature angle relative to a bone of a patient for a procedure. In certain embodiments, determining the recommended location may employ advanced computer analysis system, expert systems, machine learning, and/or automated/artificial intelligence. In another embodiment, the method 300 may include determining one or more alternative recommended locations and/or trajectory angles for instrumentation.

Next, the method 300 may proceed and a preliminary guide model is provided 306 from a repository of template instrumentation models. A preliminary guide model is a model of a preliminary guide.

As used herein, “preliminary guide” refers to a guide configured, designed, and/or engineered to serve as a template, prototype, archetype, or starting point for creating, generating, or fabricating a patient-specific guide. In one aspect, the preliminary guide may be used, as-is, without any further changes, modifications, or adjustments and thus become a patient-specific guide. In another aspect, the preliminary guide may be modified, adjusted, or configured to more specifically address the goals, objectives, or needs of a patient or a surgeon and by way of the modifications become a patient-specific guide. The patient-specific guide can be used by a user, such as a surgeon, to guide steps in a surgical procedure, such as an osteotomy. Accordingly, a preliminary guide model can be used to generate a patient-specific guide. The patient-specific guide model may be used in a surgical procedure to facilitate one or more steps of the procedure, and may be used to generate a patient-specific guide that can be used in a surgical procedure for the patient.

In certain embodiments, the preliminary guide model may be generated based on anatomic data and/or a bone model or a combination of these, and no model or predesigned structure, template, or prototype. Alternatively, or in addition, the preliminary guide model may be, or may originate from, a template guide model selected from a set of template guide models. Each model in the set of template guide models may be configured to fit for an average patient's foot. The template guide model may subsequently be modified or revised by an automated process or manual process to generate the preliminary guide model used in this disclosure.

As used herein, “template guide” refers to a guide configured, designed, and/or engineered to serve as a template for creating, generating, or fabricating a patient-specific guide. In one aspect, the template guide may be used, as-is, without any further changes, modifications, or adjustments and thus become a patient-specific guide. In another aspect, the template guide may be modified, adjusted, or configured to more specifically address the goals, objectives, or needs of a patient or a surgeon and by way of the modifications become a patient-specific guide. The patient-specific guide can be used by a user, such as a surgeon, to guide making one or more resections of a structure, such as a bone for a procedure. Accordingly, a template guide model can be used to generate a patient-specific guide model. The patient-specific guide model may be used in a surgical procedure to address, correct, or mitigate effects of the identified deformity and may be used to generate a patient-specific guide that can be used in a surgical procedure for the patient.

Next, the method 300 may register 308 the preliminary guide model with one or more bones of the bone model. This step 308 facilitates customization and modification of the preliminary guide model to generate a patient-specific guide model from which a patient-specific guide can be generated. The registration step 308 may combine two models and/or patient imaging data and positions both models for use in one system and/or in one model.

Next, the method 300 may design 310 a patient-specific guide model based on the preliminary guide model. The design step 310 may be completely automated or may optionally permit a user to make changes to a preliminary guide model or partially completed patient-specific guide model before the patient-specific guide model is complete. A preliminary guide model and patient-specific guide model are two examples of an instrument model. As used herein, “instrument model” refers to a model, either physical or digital, that represents an instrument, tool, apparatus, or device. Examples of an instrument model can include a cutting guide model, a resection guide model, an alignment guide model, a reduction guide model, a patient-specific tendon trajectory guide model, and the like. In one embodiment, a patient-specific guide and a patient-specific guide model may be unique to a particular patient and that patient's anatomy and/or condition.

The method 300 may conclude by a step 312 in which patient-specific guide may be manufactured based on the patient-specific guide model. Various manufacturing tools, devices, systems, and/or techniques can be used to manufacture the patient-specific guide.

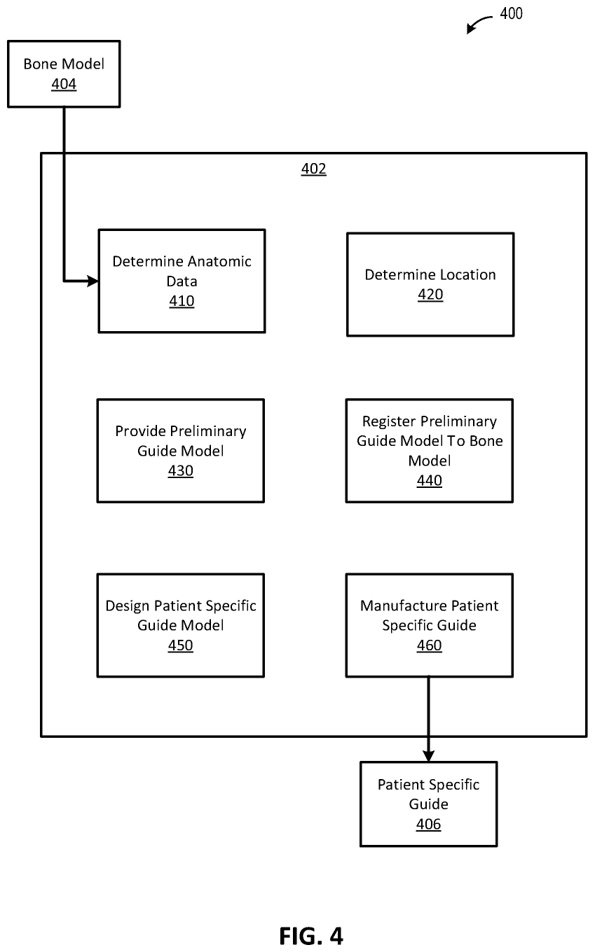

FIG. 4 illustrates an exemplary system 400 configured to generate one or more patient-specific instruments configured to facilitate surgical procedures, according to one embodiment. The system 400 may include an apparatus 402 configured to accept, review, receive or reference a bone model 404 and provide a patient-specific guide 406. In one embodiment, the apparatus 402 is a computing device. In another embodiment, the apparatus 402 may be a combination of computing devices and/or software components or a single software component such as a software application.

The apparatus 402 may include a determination module 410, a location module 420, a provision module 430, a registration module 440, a design module 450, and a manufacturing module 460. Each of which may be implemented in one or more of software, hardware, or a combination of hardware and software.

The determination module 410 determines anatomic data 412 from a bone model 404. In certain embodiments, the system 400 may not include a determination module 410 if the anatomic data is available directly from the bone model 404. In certain embodiments, the anatomic data for a bone model 404 may include data that identifies each anatomic structure within the bone model 404 and attributes about the anatomic structure. For example, the anatomic data may include measurements of the length, width, height, and density of each bone in the bone model. Furthermore, the anatomic data may include position information that identifies where each structure, such as a bone is in the bone model 404 relative to other structures, including bones. The anatomic data may be in any suitable format and may be stored separately or together with data that defines the bone model 404.

In one embodiment, the determination module 410 may use advanced computer analysis system such as image segmentation to determine the anatomic data. The determination module 410 may determine anatomic data from one more sources of medical imaging data, images, files, or the like. Alternatively, or in addition the determination module 410 may use software and/or systems that implement one or more artificial intelligence methods (e.g., machine learning and/or neural networks) for deriving, determining, or extrapolating, anatomic data from medical imaging or the bone model. In one embodiment, the determination module 410 may perform an anatomic mapping of the bone model 404 to determine each unique aspect of the intended osteotomy procedure and/or bone resection and/or bone translation. The anatomic mapping may be used to determine coordinates to be used for an osteotomy procedure, position and manner of resections to be performed either manually or automatically or using robotic surgical assistance, a width for bone cuts, an angle for bone cuts, a predetermined depth for bone cuts, dimensions and configurations for resection instruments such as saw blades, milling bit size and/or speed, saw blade depth markers, and/or instructions for automatic or robotic resection operations.

In one embodiment, the determination module 410 may use advanced computer analysis system such as image segmentation to determine the anatomic data. The determination module 410 may determine anatomic data from one or more sources of medical imaging data, images, files, or the like. The determination module 410 may perform the image segmentation using 3D modeling systems and/or artificial intelligence (AI) segmentation tools. In certain embodiments, the determination module 410 is configured to identify and classify portions of bone based on a condition of the bone, based on the bone condition. Such classifications may include identifying bone stability, bone density, bone structure, bone deformity, bone structure, bone structure integrity, and the like. Accordingly, the determination module 410 may identify portions or sections or one or more bones based on a quality metric for the bone. Advantageously, that determination module 410 can identify high quality bone having a viable structure, integrity, and/or density versus lower quality bone having a nonviable structure, integrity, and/or density and a plurality of bone quality levels in between.

Accordingly, the determination module 410 can guide a surgeon to determine which areas of one or more bones of a patient are within a “soft tissue envelope” (bone of undesirable quality) as that bone relates to a particular deformity or pathology. Identifying the quality of one or more bones of the patient can aid a surgeon in determining what type of correction or adjustment is needed. For example, an ulceration that occurs due to a boney deformity can be mapped using the determination module 410 in a way that a correction can be performed to correct the deformity and reduce pressure to an area and address the structures that were causing the pressure ulceration/skin breakdown.

In addition, the determination module 410 and/or another component of the apparatus 402 can be used to perform anatomic mapping which may include advanced medical imaging, such as the use of CT scan, ultrasound, MRI, and bone density scans can be combined to effectively create an anatomic map that determines the structural integrity of the underlying bone.

Identifying the structural integrity of the underlying bone can help in determining where bone resections can be performed to preserve the densest bone in relation to conditions such as Charcot neuropathic, arthropathy where lesser dense bone can fail and collapse. It is well documented in the literature that failure to address and remove such lesser dense bone can ultimately lead to failure of a reconstruction and associated hardware.

The present disclosure provides, by way of at least the exemplary system 400, an anatomic map that can be part of anatomic data. The anatomic map can combine structural, deformity, and bone density information and can be utilized to determine the effective density of bone and help to determine where bone should be resected in order to remove the lesser dense bone while maintaining more viable bone to aid in the planning of the osteotomy/bone resection placement.

The location module 420 determines or identifies one or more recommended locations and/or trajectory angles for deployment of an instrument, graft, and/or soft tissue based on the anatomic data 412 and/or the bone model 404. In one embodiment, the location module 420 may compare the anatomic data 412 to a general model that is representative of most patient's anatomies and may be free from deformities or anomalies The location module 420 can operate autonomously and/or may facilitate input and/or revisions from a user. The location module 420 may be completely automated, partially automated, or completely manual. A user may control how automated or manual the determining of the recommended location and/or trajectory angles is.

The provision module 430 is configured to provide a preliminary guide model 438. The provision module 430 may use a variety of methods to provide the preliminary guide model. In one embodiment, the provision module 430 may generate a preliminary guide model. In the same, or an alternative embodiment, the provision module 430 may select a template guide model for a surgical procedure configured to enable locating the position for one or more instruments and/or providing a trajectory provided by the location module 420. In one embodiment, the provision module 430 may select a template guide model from a set of template guide models (e.g., a library, set, or repository of template guide models).

The registration module 440 registers the preliminary guide model with one or more bones or other anatomical structures of the bone model 404. As explained above, registration is a process of combining medical imaging data, patient imaging data, and/or one or more models such that the preliminary guide model can be used with the bone model 404.

The design module 450 designs a patient-specific guide (or patient-specific guide model) based on the preliminary guide model. The design operation of the design module 450 may be completely automated, partially automated, or completely manual. A user may control how automated or manual the designing of the patient-specific guide (or patient-specific guide model) is.

The manufacturing module 460 may manufacture a patient-specific guide 406 using the preliminary guide model. The manufacturing module 460 may use a patient-specific guide model generated from the preliminary guide model. The manufacturing module 460 may provide the patient-specific guide model to one or more manufacturing tools and/or fabrication tool. The patient-specific guide model may be sent to the tools in any format such as an STL file or any other CAD modeling or CAM file or method for data exchange. In one embodiment, a user can adjust default parameters for the patient-specific guide such as types and/or thicknesses of materials, dimensions, and the like before the manufacturing module 460 provides the patient-specific guide model to a manufacturing tool.

Effective connection of the guide to one or more bones can ensure that surgical steps are performed in desired locations and/or with desired orientations and mitigate undesired surgical outcomes.

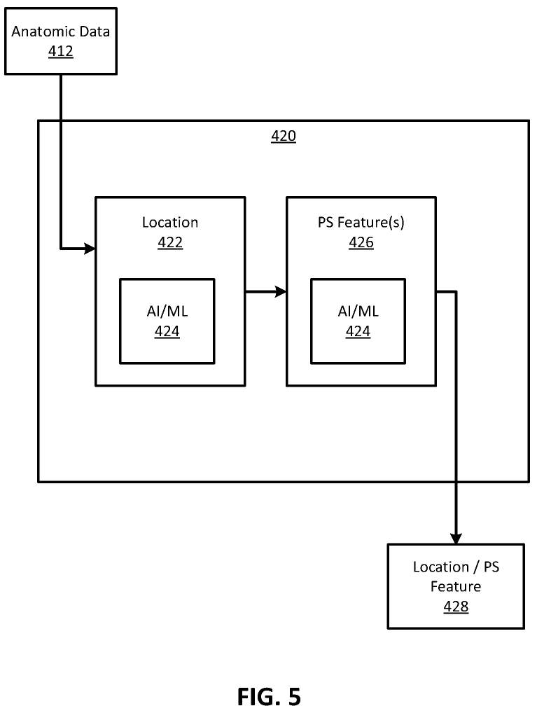

FIG. 5 illustrates an exemplary location module 420 configured to determine a recommended location and/or trajectory for steps and/or instruments in a surgical procedure, according to one embodiment. The location module 420 may factor in one or more landmarks on one or more surfaces of one or more bones of a patient of the bone model 404. The location module 420 may be completely automated, partially automated, or completely manual. A user may control how automated or manual the determination of the recommended location is. The user may provide instructions to the location module 420 to facilitate automatic or partially automated determination of one or more recommended locations.

The location module 420 may include a location module 422. The location module 422 may be configured for automated determination of a recommended location for steps and/or instruments in a surgical procedure. For example, in one embodiment, the location module 422 includes an artificial intelligence or machine learning module 424. The artificial intelligence or machine learning module 424 is configured to implement one or more of a variety of artificial intelligence modules that may be trained for identifying bones in the bone model 404, determining surfaces and/or sides of one or more bones, determining landmarks (both natural and/or abnormalities), determining axes of a bone, such as a longitudinal axis and/or a horizontal axis of a bone based on anatomic data 412 and/or a bone model 404. In another embodiment, the location module 420 may receive patient imaging data, a bone model, a CAD model or the like and use these inputs to determine a recommended location and/or trajectory in relation to one or more bones of a patient.

In one embodiment, the artificial intelligence or machine learning module 424 may be trained using a large data set of anatomic data 412 for healthy bones and a large data set of anatomic data 412 for bones with abnormalities and/or landmarks in which the abnormalities and/or landmarks have been previously identified and labeled in the dataset. The artificial intelligence or machine learning module 424 may implement, or use, a neural network configured according to the training such that as the artificial intelligence or machine learning module 424 accepts the anatomic data 412 for a particular patient, the artificial intelligence or machine learning module 424 is able to determine one or more recommended locations (e.g., a recommended location and one or more alternative locations for a guide, step, or instrument).

The location module 422 may interact with a patient specific feature module 426. The patient specific feature module 426 may take one or more recommended locations provided by the location module 422 and the bone model 404 and/or anatomic data 412 and determine suitable patient specific features. In certain embodiments, the patient specific features provided by the patient specific feature module 426 may include a number of resection features, an angle or trajectory for one or more resection features, a number, size, and/or position of bone attachment features, a number, size, or position of alignment guides or a combination of these. In certain embodiments, the patient specific feature module 426 may focus on resection features.

As with the location module 420, the patient specific feature module 426 may be completely automated, partially automated, or completely manual. A user may control how automated or manual the determination of one or more trajectories is. The user may provide instructions to the patient specific feature module 426 to facilitate automatic or partially automated determination of one or more trajectories. In one embodiment, the location module 422 includes an artificial intelligence or machine learning module 424 that facilitates determining one or more trajectories.

The location module 420 outputs a recommended location and/or patient specific feature 428 for a surgical procedure.

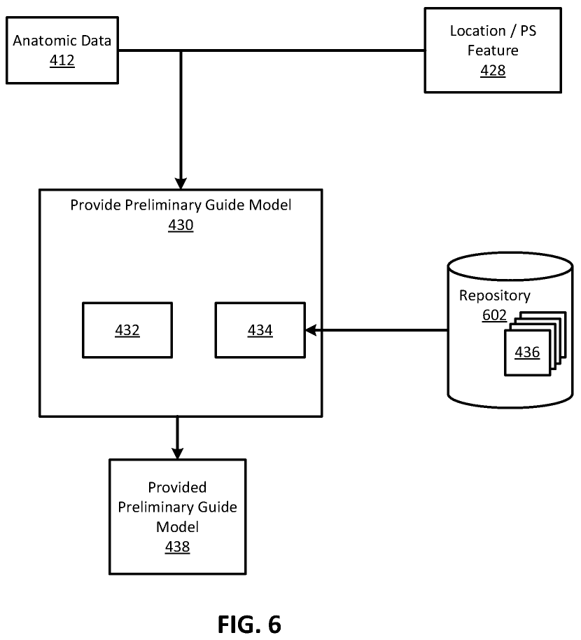

FIG. 6 illustrates an exemplary provision module 430 configured to provide a preliminary guide model, according to one embodiment. The provision module 430 may accept anatomic data 412 and a location/patient specific feature 428. In the illustrated embodiment, the provision module 430 may generate a preliminary guide model 438 (e.g., generate from ‘scratch’) or the provision module 430 may select a template guide model 436 automatically from a set of template guide models 436 stored in a repository 602. The provision module 430 may incorporate a variety of parameters in order to provision, generate, determine, or select a template guide model 436. For example, in addition to the anatomic data 412, the provision module 430 may include patient imaging data, deformity parameters for a variety of angular deformities (in all 3 planes) of the midfoot or hind foot and ankle, patient preferences, and/or surgeon input parameters.

In one embodiment, the provision module 430 may include a generator 432 and/or a selection module 434. In one embodiment, the generator 432 is configured to generate a preliminary guide model 438. In certain embodiments, the generator 432 may generate or create the preliminary guide model based on anatomic data and/or a bone model or a combination of these and no other inputs. (e.g. no model or predesigned structure, template, or prototype). Alternatively, or in addition, the generator 432 may generate or create the preliminary guide model using a standard set of features or components that can be combined to form the preliminary guide model. The generated preliminary guide model may subsequently be modified or revised by an automated process, and/or manual process, to generate the preliminary guide model used in this disclosure.

The selection module 434 may be configured to select a template guide model 436 for an osteotomy procedure configured to correct the deformity identified by the location module 420. In one embodiment, the provision module 430 may select a template guide model 436 from a set of template guide models 436 (e.g., a library, set, or repository of template guide models 436). In one embodiment, the template guide model 436 may include digital models. In another embodiment, the template guide model 436 may include physical models. In such an embodiment, the repository 602 may be a warehouse or other inventory repository. Where the template guide model 436 are physical models, the systems, modules, and methods of this disclosure can be used and the physical model may be milled or machined (e.g., a CNC machine) to form a patient-specific guide that conforms to the bone surfaces of the patient.

Selection of a suitable template guide model 436 may be completely automated and/or may be partially automated and/or may depend on confirmation from a user before a generated preliminary guide model or a proposed template guide model 436 becomes the preliminary guide model 438. In another embodiment, the selection module 434 may facilitate a manual selection by a user of a template guide model 436 that would become the preliminary guide model 438. The selection module 434 may use the anatomic data 412 or the bone model 404 or a combination of these to select a suitable template guide model that would become the preliminary guide model 438.

In another embodiment, the generator 432 may facilitate revisions or edits by a user of a generated guide model that will become the preliminary guide model 438. The selection module 434 may use the anatomic data 412 or the bone model 404 or a combination of these to select a suitable template guide model that would become the preliminary guide model 438.

The repository 602 may include any number of, and/or a variety of template guide models 436. The template guide models 436 may be distinguished based on a gender or age of the patient, which joint of a midfoot, hind foot, or ankle will be cut, which material will be used for the template guide, and the like. The template guide model 436 may differ from each other in what degree of deformity correction the template guide model 436 is designed to provide. In addition, the template guide models 436 may be distinguished based how one or more features of the template guide model 436 are positioned, arranged, and/or configured relative to each other. For example, in certain template guide models 436, the number, position, and/or configuration of alignment features and/or bone attachment features (e.g., holes) may vary based on needs or preferences of patients, the nature of the deformity, and/or surgeon preferences.

In certain embodiments, the template guide models 436 may vary in how the slots (e.g., resection features) for the cuts are positioned, angled, and oriented relative to each other and/or to a longitudinal axis of respective bones at a joint for use with the template guide model 436. For example, in one template guide model 436 the slot 1352 for a resection of a metatarsal bone may be perpendicular to a longitudinal axis of the metatarsal bone and the slot 1350 may be angled relative to a longitudinal axis of the cuneiform or cuboid bone such that once the two bones are brought together the deformity is corrected. Alternatively, in another template guide model 436 the slot for a resection of a metatarsal bone may be angled relative to a longitudinal axis of the metatarsal bone and the slot 1350 may be perpendicular to a longitudinal axis of the cuneiform or cuboid bone such that once the two bones are brought together the deformity is corrected.

The selection module 434 may be configured to automatically select a template guide model 436 and/or provide an automatic template guide model 436 recommendation that can be changed by a user, such as a surgeon. For example, in one embodiment, the provision module 430 and/or selection module 434 includes an artificial intelligence or machine learning module. The artificial intelligence or machine learning module is configured to implement one or more of a variety of artificial intelligence modules that may be trained for selecting a template guide model 436 based on anatomic data 412 and/or other input parameters. In one embodiment, the artificial intelligence or machine learning module may be trained using a large data set of anatomic data 412 for suitable template guide models 436 identified and labeled in the dataset by professionals for use to treat a particular deformity. The artificial intelligence or machine learning module may implement, or use, a neural network configured according to the training such that as the artificial intelligence or machine learning module is able to select a suitable template guide model 436. The template guide model 436 selected by the selection module 434 can become the preliminary guide model 438.

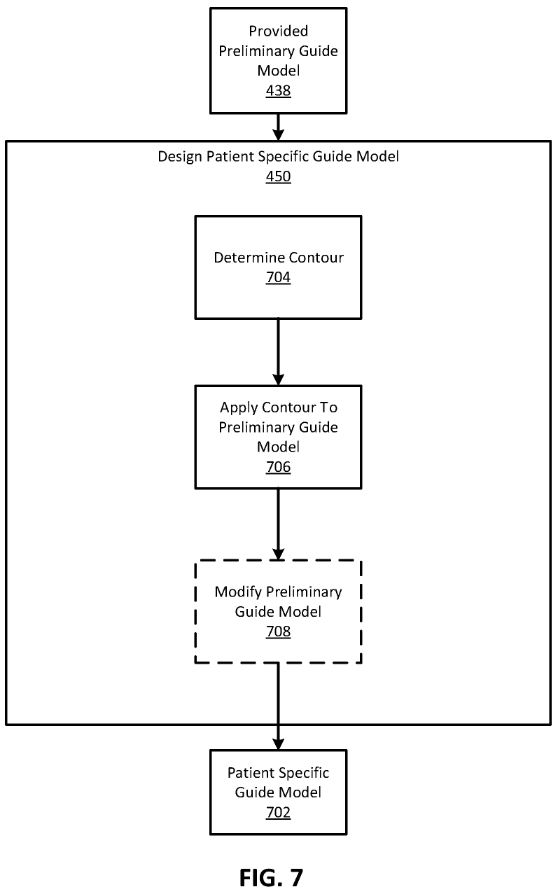

FIG. 7 illustrates an exemplary design module 450 configured to design a patient-specific guide model, according to one embodiment. The design module 450 may accept a preliminary guide model 438 and generate a patient-specific guide model 702. In one embodiment, the design module 450 includes a contour module 704, an application module 706, and/or an optional modification module 708.

Referring now to FIG. 7, the design module 450 may modify the preliminary guide model 438 such that the bone-facing and/or bone-contacting surfaces of the preliminary guide model 438 match a contour of the surfaces and/or joint of one or more bones where a step of an orthopedic procedure will be performed using the preliminary guide model 438.

The contour module 704 may determine a contour of the bones that will contact the preliminary guide model 438. The contour module 704 may use a bone model 404 and/or anatomic data 412 to determine the contour. For example, the contour module 704 may determine the shape of a dorsal surface of a calcaneus 222.

The application module 706 may apply the contour to the provided preliminary guide model 438 to custom contour a bone engagement surface of the preliminary guide model 438 to match the shape, contour, and/or one or more landmarks of a bone, such as a dorsal surface of a calcaneus 222. Applying the contour to the preliminary guide model 438 may convert the preliminary guide model 438 to a patient-specific guide model 702.

Generation of the contours of bone engagement surface of the preliminary guide model 438 may be performed in various CAD programs In some embodiments, the shapes of the corresponding surface dorsal surface of a calcaneus 222 may be obtained directly from the bone model 404, anatomic data 412, CAD models and/or CT scan data, and simply copied onto the preliminary guide model 438. Various operations may be used to copy surfaces from one object to another. Additionally, or alternatively, various Boolean operations, such as a Boolean subtraction operation, may be used to remove material from a model for the body of the preliminary guide model 438 with a shape that matches the dorsal surface of a calcaneus 222.

In certain embodiments, the design module 450 may include an optional module, such as a modification module 708. The modification module 708 may enable a user such as a technician or surgeon to make additional modifications to the design and configuration of the preliminary guide model 438. In one embodiment, the user can change any of the features, trajectories, fixation holes, handle engagement holes, angles, configurations, or parameters of the preliminary guide model 438. For example, a surgeon may be aware of other concerns or anatomic aspects of a patient, for example on an opposite foot or in connection with a hip or other orthopedic joint which motivate the surgeon to adjust an angle of one of more trajectories of the preliminary guide model 438.

Alternatively, or in addition, a user may use the modification module 708 to modify instrumentation such as a guide. The user may add, remove, or modify steps and/or the instrumentation to create a patient-specific surgical procedure. In this manner, a user may configure features of a preliminary guide model 438 or modified preliminary guide model to a patient-specific osteotomy procedure the surgeon is planning for the patient.

The user may review the preliminary guide model 438 and make adjustments or revisions or make no adjustments or revisions. The output of the modification module 708 and/or the application module 706 is a patient-specific guide model 702.

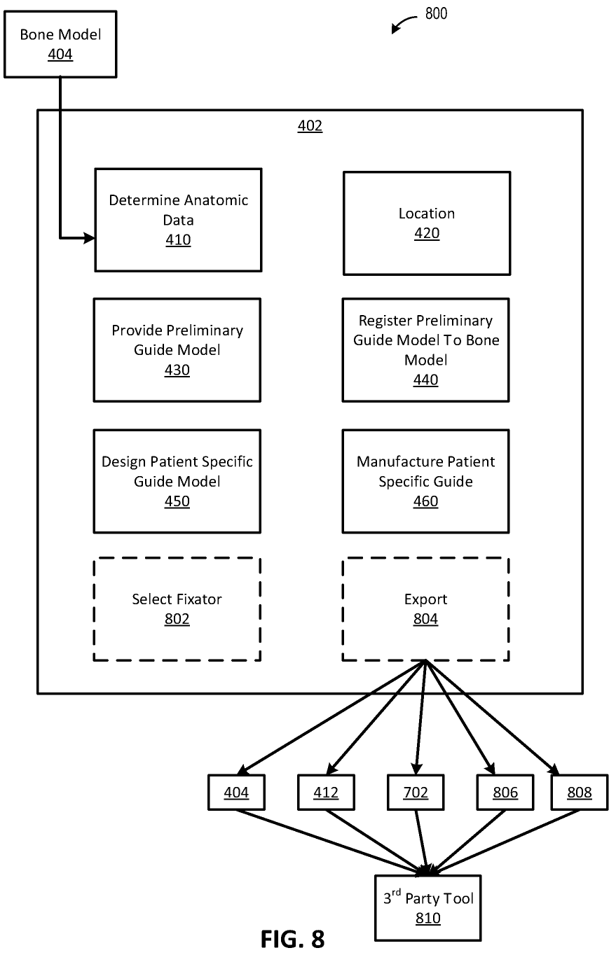

FIG. 8 illustrates an exemplary system 800 configured to generate one or more patient-specific instruments configured to correct a bone condition, according to one embodiment. The system 800 may include similar components or modules to those described in relation to FIG. 4. In addition, the system 800 may include a fixator selector 802 and/or an export module 804.

The fixator selector 802 enables a user to determine which fixator(s) to use for a surgical procedure planned for a patient. In one embodiment, the fixator selector 802 may recommend one or more fixators based on the bone model 404, the location, the trajectory, or input from a user or a history of prior surgical procedures performed. The fixator selector 802 may select a fixator model from a set of predefined fixator models or select a physical fixator from a set of fixators. The fixators may include a plate and associated accessories such as screws, anchors, and the like.

In one embodiment, the fixator selector 802 includes an artificial intelligence or machine learning module. The artificial intelligence or machine learning module is configured to implement one or more of a variety of artificial intelligence modules that may be trained for selecting fixator(s) based on anatomic data 412 and/or other input parameters. In one embodiment, the artificial intelligence or machine learning module may be trained using a large data set of anatomic data 412 for suitable fixator(s) identified and labeled in the dataset by professionals for use to treat a particular condition. The artificial intelligence or machine learning module may implement, or use, a neural network configured according to the training such that as the artificial intelligence or machine learning module is able to select or recommend suitable fixator(s).

The export module 804 is configured to enable exporting of a patient-specific guide model 702 for a variety of purposes including, but not limited to, fabrication/manufacture of a patient-specific guide 406 and/or fixator(s), generation of a preoperative plan, generation of a physical bone model matching the bone model 404, and the like. In one embodiment, the export module 804 is configured to export the bone model 404, anatomic data 412, a patient-specific guide model 702, a preoperative plan 806, a fixator model 808, or the like. In this manner the custom instrumentation and/or procedural steps for a surgical procedure can be used in other tools. The preoperative plan 806 may include a set of step-by-step instructions or recommendation for a surgeon or other staff in performing a surgical procedure such as an osteotomy. The preoperative plan 806 may include images and text instructions and may include identification of instrumentation to be used for different steps of the surgical procedure. The instrumentation may include the patient-specific guide 406 and/or one or more fixators. In one embodiment, the export module 804 may provide a fixator model which can be used to fabricate a fixator for the surgical procedure.

The exports (404, 412, 702, 806, and 808) may be inputs for a variety of 3rd party tools 810 including a manufacturing tool, a simulation tool, a virtual reality tool, an augmented reality tool, an operative procedure simulation tool, a robotic assistance tool, and the like. A surgeon can then use these tools when performing a surgical procedure or for rehearsals and preparation for the surgical procedure. For example, a physical model of the bones, patient-specific guide 406, and/or fixators can be fabricated, and these can be used for a rehearsal operative procedure. Alternatively, a surgeon can use the bone model 404, preliminary guide model 438, and/or a fixator model to perform a simulated surgical procedure using an operative procedure simulation tool.

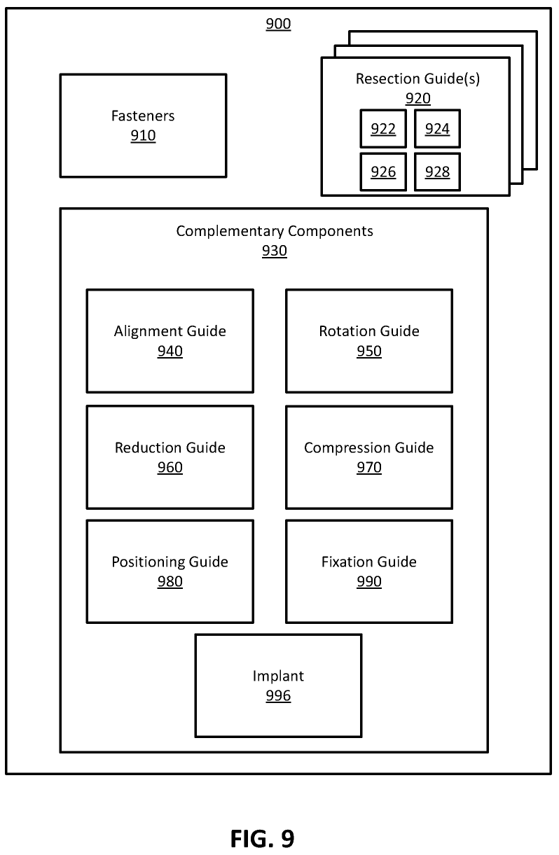

FIG. 9 illustrates an exemplary system 900, according to one embodiment. The system 900 can include one or more fasteners 910, one or more resection guides 920, and one or more complementary components 930. While a system 900 can be used for a variety of procedures, one or more features, components, and/or aspects of the system 900 may be particularly suited for one or more osteotomies on one or more bones of a structure such as a patient's foot, ankle, wrist, hand, shoulder, or the like.

In certain embodiments, the one or more fasteners 910 can include both, one or more permanent fasteners and one or more temporary fasteners. Typically, the fasteners 910 may be used during a variety of different steps of a procedure. Temporary fasteners are often used because they can securely hold bone or parts of bones while steps of the procedure are conducted. A common temporary fastener that can be used with system 900 is a K-wire, also referred to as a pin.

The one or more resection guides 920 assist a surgeon in performing different resection steps for an osteotomy procedure. In certain embodiments, a resection guide 920 includes one or more resection features 922 and one or more bone attachment features 924. The resection features 922 can take a variety of forms and/or embodiments Similarly, the bone attachment features 924 can take a variety of forms and/or embodiments. The resection features 922 provide a guide for a surgeon using a cutting tool to resect a bone, one or more bones, or other tissues of a patient. The bone attachment features 924 serve to secure the resection guide 920 to one or more bones and/or one or more other structures. Often, a bone attachment feature 924 can include a hole in the resection guide 920 together with a temporary fastener such as a K-wire or pin.

The bone attachment features 924 facilitate attachment of a resection guide 920 to one or more bones, or bone fragments, of a patient. The bone attachment features 924 may include any of a wide variety of fasteners including, but not limited to, holes, spikes, fastening devices, and/or the like. Effective connection of the resection guide 920 to one or more bones across a joint can ensure that cut surfaces are formed in desired locations and orientation and mitigate removal of hard tissue and/or soft tissue in undesired locations.

In certain embodiments, a resection guide 920 may include one or more bone engagement surfaces 926 and/or one or more landmark registration features 928. In certain embodiments, a landmark registration feature 928 may extend from one or more sides of the resection guide 920 and engage with one or more landmarks of a bone of a patient. Registration of the landmark registration feature 928 to the landmark of the bone can serve to confirm that a surgeon has located a desired placement and/or orientation for a resection guide 920.

In certain embodiments, the bone engagement surfaces 926 are patient-specific: contoured to match a surface of one or more bones the resection guide 920 contacts during the procedure. Alternatively, or in addition, the bone engagement surface 926 may not be patient-specific and may, or may not, contact a bone surface during use of the resection guide 920. Those of skill in the art appreciate that one or more sides of any of the members of the system 900 may include one or more bone engagement surfaces 926. Consequently, one or more sides of the fasteners 910, the resection guide(s) 920, the complementary components 930, and/or the implants 996 may include one or more bone engagement surfaces 926.

The complementary components 930 serve to assist a surgeon during one or more steps of a procedure. Those of skill in the art appreciate that a number of components can serve as complementary components 930. One or more of the features, functions, or aspects of the complementary components 930 can include patient-specific features.

Examples of complementary components 930 include, but are not limited to, an alignment guide 940, a rotation guide 950, a reduction guide 960, a compression guide 970, a positioning guide 980, a fixation guide 990, and/or one or more implants 996. In general, the complementary components 930 serve to assist a surgeon in performing the function included in the name of the complementary component 930. Thus, an alignment guide 940 can help a surgeon align bones, parts of bones, or other parts of a patient as part of a procedure. A rotation guide 950 can help a surgeon rotate one or more bones, parts of bones, or other parts of a patient as part of a procedure.

A reduction guide 960 can help a surgeon position and/or orient one or more bones, parts of bones, or other parts of a patient as part of a procedure in order to reduce the bone, bones, bone parts, or other parts and/or in order to position and/or orient the bone, bones, bone parts, or other parts to a desired position and/or orientation. A compression guide 970 can help a surgeon compress one or more bones, parts of bones, or other parts of a patient together or against an implant as part of a procedure. A positioning guide 980 can help a surgeon position one or more bones, parts of bones, or other parts of a patient as part of a procedure.

In certain embodiments, the positioning guide 980 may be designed and fabricated to be patient-specific. The patient-specific aspects can include a patient-specific bone engagement surface, a predefined angle for reorienting one or more bone or bone parts within one or more planes, a predefined position for bone attachment features 924 or fasteners 910, or the like. Alternatively, or in addition, the positioning guide 980 may be selected from a kit, collection, or repository of a number of positioning guides 980: each having a different configuration for one or more aspects/attributes of the positioning guide 980. For example, each member of the repository/kit may include a different positioning angle (repositioning or correction angle), the angles may differ by 2 degrees for example. In such an embodiment, each positioning guide 980 may not be patient-specific to a particular patient but may provide the desired amount of positioning to meet the goals of the surgeon. In certain embodiments, a preoperative plan generated based on the present disclosure may include a recommendation for the positioning guide 980 to be used, even if the recommended positioning guide 980 is not patient-specific to the particular patient.

A fixation guide 990 can help a surgeon in completing one or more temporary or permanent fixation steps for one or more bones, parts of bones, or other parts of a patient as part of a procedure. The fixation guide 990 may include and/or may use one or more components of a fastener or fixation system including implant hardware of the fastener or fixation system.

One example of a complementary components 930 may include a compressor/distractor. The compressor/distractor can be used to compress or distract bones or parts of bones involved in a procedure.

Advantageously, the system 900 can help a surgeon overcome one or more of the challenges in performing an osteotomy procedure, particularly on bones of a hand or of a foot of a patient, such as on the forefoot, midfoot, or hindfoot. One challenge during an osteotomy procedure can be maintaining control of, and/or position, and/or orientation of a bone, one or more bones, and/or bone pieces/fragments, particularly once a resection or dissection is performed. Advantageously, the fasteners 910, resection guide(s) 920, and/or complementary components 930 can be configured to assist in overcoming this challenge.

Advantageously, the system 900 can help a surgeon in positioning, placing, and/or orienting a resection guide accurately. Modern techniques may include preoperative planning, simulation, or even practice using computer models, 3D printed models, virtual reality systems, augmented reality systems or the like. However, simulations and models are still different from actually positioning a resection guide on a patient's bone, joint, or body part during the procedure. The system 900 can include a number of features, including patient-specific features, to assist the surgeon with the positioning. In one embodiment, the resection guide 920 can include one or more landmark registration features 928.

Advantageously, the system 900 can help a surgeon in securing guides of the osteotomy system 900, such as a resection guide, as well as how to readily remove the guide (e.g., resection guide) without disturbing a reduction, shifting, reorienting, or repositioning one or more bones or parts of bones while removing the guide. In certain embodiments, the system 900 is configured to permit removal of a guide while keeping temporary fasteners in place for use in subsequent steps of an osteotomy procedure. Alternatively, or in addition, the system 900 facilitates positioning of temporary fasteners during one step of the surgical procedure for use in a subsequent step of the surgical procedure. Removal of a guide during an osteotomy procedure can be particularly challenging where translation and/or rotation of the bones involved in the surgical procedure is required for the success of the surgical procedure. Advantageously, the system 900 accommodates translation and/or rotation of the bones during the surgical procedure while facilitating a successful outcome for the surgical procedure.

Advantageously, the components of the system 900 can be specifically designed for a particular patient. Alternatively, or in addition, the components of the system 900 can be specifically designed for a class of patients. Each of the components of the system 900 can be designed, adapted, engineered and/or manufactured such that each feature, attribute, or aspect of the component is specifically designed to address one or more specific indications present in a patient. Advantageously, the cuts made for the osteotomy procedure can be of a size, position, orientation, and/or angle that provides from an optimal osteotomy with minimal risk of undesirable resection. In one embodiment, the components of the system 900 can be configured such that an osteotomy is performed that enables a correction in more than one plane in relation to the parts of the body of the patient. For example, cut channels in a resection guide 920 can be oriented and configured such that when the bones are fused/fixated the correction results from translation, rotation, and/or movement of bones or bone parts in two or more planes (e.g., sagittal and transverse).

In certain embodiments, the exemplary system 900 may include a plurality of fasteners 910, resection guides 920, and/or complementary components 930. For example, a surgeon may plan to resect and/or dissect one or more bone(s) in order to accomplish a desired correction. One or more bone fragments may be resected from a distal end of a patient's long bone. Positioning the cuts for the osteotomy and angling them (e.g., setting an appropriate trajectory) for optimal clearance to tissue covered by the resected bone can be a challenge.

A surgeon may use one or more components in an exemplary system 900 for a surgical procedure. The one or more cuts made using the exemplary system 900 to provide a desired correction. Each of the components of the exemplary system 900 can be identified, defined, and reviewed using the apparatuses, systems, and/or methods of the present disclosure.

In certain embodiments, the components of the system 900 may be made as small as possible to minimize the amount of soft tissue that is opened in the patient for the osteotomy procedure. Alternatively, or in addition, walls and/or sides of the components may be beveled and/or angled to avoid contact with other hard tissue or soft tissues in the operating field for the osteotomy procedure.

Those of skill in the art will appreciate that for certain surgical procedures a complementary component 930 may not be needed or a given complementary component 930 may be optional for use in the surgical procedure. Similarly, those of skill in the art will appreciate that certain features of the fasteners 910, resection guides 920, and/or complementary components 930 can be combined into one or more of apparatus or devices or may be provided using a plurality of separate devices.

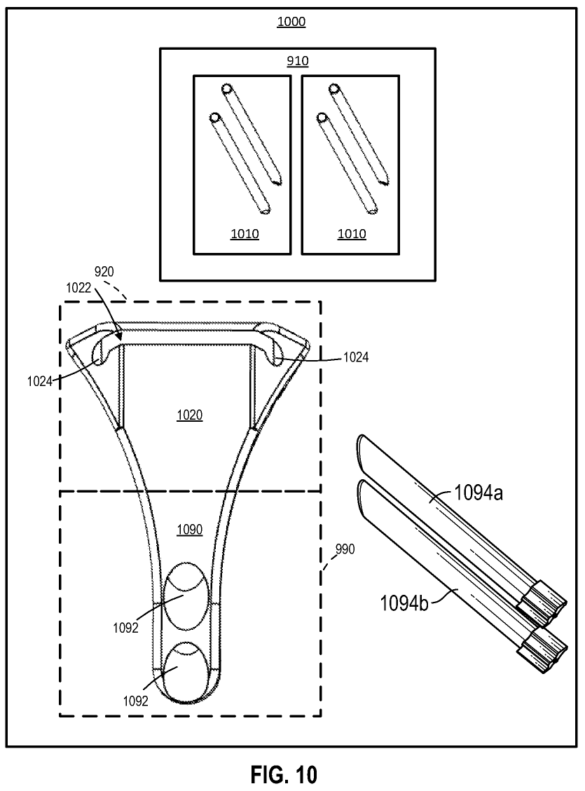

FIG. 10 illustrates an exemplary system 1000, according to one embodiment. The system 1000 may include one or more fasteners 910, such as fasteners 1010, one or more resection guides 920, such as resection guide 1020, and may include one or more other complementary components 930, such as a fixation guide 990, such as fixation guide 1090. The osteotomy system 1000 can be used for a variety of surgical procedures.

In another embodiment, the osteotomy system 1000 includes a fixation guide 1090 without a resection guide 1020. Such an embodiment may include a similar size and shape and features as a combined resection guide 1020 and fixation guide 1090 illustrated in FIG. 10 however this embodiment of fixation guide 1090 may not include a resection feature 1022. Said another way, the fixation guide 1090 and resection guide 1020 may be separate and distinct in one embodiment. Alternatively, or in addition, the resection guide 1020 and fixation guide 1090 can be connected and/or configured to be coupled to each other.

In one embodiment, the osteotomy system 1000 is used to remediate a condition present in a patient. For example, in one surgical procedure a surgeon may dissect a distal end of a tibia (e.g., a malleolus) from the tibia and retract the malleolus in order to gain access to a talus and perform a procedure on the talus. The osteotomy system 1000 can include a resection guide 1020 and a fixation guide 1090. Alternatively, or in addition, the osteotomy system 1000 can include just a resection guide 1020 or just a fixation guide 1090.

In the illustrated embodiment, the resection guide 1020 includes one or more resection features 1022. A resection feature 1022 guides a surgeon in performing a resection or dissection of a bone by facilitating keeping a cutting tool in line with, and/or within the resection feature 1022 (at a desired trajectory relative to one or more bones of the patient). In one embodiment, the resection feature 1022 may guide resection of the bone to dissect the bone into a proximal fragment and a distal fragment. In the context of dissecting a malleolus from a tibia, the proximal fragment may be the tibia and the distal fragment may be the malleolus. In the illustrated embodiments, the resection feature 1022 is shaped like a slot or a channel and includes a first end and a second end. The length, width, and shape of the resection feature 1022 may be elongated like a slot or may have one of many other shapes. In one embodiment, the resection feature 1022 may have a curved shape. In certain embodiment, the first end and/or second end may be open ended or closed ended.

In the illustrated embodiment, the resection guide 1020 may be configured for use on a medial side of an ankle of a patient, with the side visible in FIG. 10 facing away from the bone(s). The resection feature 1022 may be positioned and oriented relative to a malleolus of a tibia to facilitate forming a cut that separates the medial malleolus from the distal end of the tibia to provide access for a remediation procedure on a talus. Of course, the resection feature 1022 can also be configured to enable osteotomies for a similar procedure on the lateral malleolus of the fibula.

Those of skill in the art will appreciate that the resection guide(s) 920 may have a variety of shapes, sizes and configurations. In one embodiment, these attributes can each be customized and adapted to meet needs and/or preferences of patients, a patient's anatomy, the nature of the deformity, and/or surgeon preferences. In certain embodiments, the resection guide(s) 920 can be sized to be small enough to support minimally invasive surgery techniques and large enough to provide the surgeon the desired view of the operating field for the procedure.

In certain embodiments, the resection guide 920 include visualization features (e.g., a pattern, windows, open areas around the body of the resection guide 920, open areas around the body of the resection guide 920, and the like). The visualization features can serve as markers to assist a surgeon in knowing that the resection guide 920 is properly placed, positioned, oriented, seated, registered relative to other anatomical structures during a surgical procedure. In particular, a surgeon can confirm intraoperatively that steps of the procedure match the information, steps, and/or plan set forth in a preoperative plan. Advantageously, the features of the resection guides 920 can facilitate one or more osteotomies as well as other steps in a surgical procedure.

Advantageously, the angle of each resection feature 1022 (e.g., the angle the resection feature 1022 extends through a body of the resection guide 1020) relative to one or more bones (or bone surfaces or other reference points, reference lines, and/or reference planes) can be determined, selected, or indicated by a surgeon before the resection guide 1020 is fabricated Similarly, a surgeon can designate an angle of the resection feature 1022 relative to the talus or talar dome. In certain embodiments, the angle of the resection feature 1022 relative to the talus or talar dome may be about 45 degrees. In addition, a surgeon can designate whether sides of the resection feature 1022 are open and/or closed.

In the illustrated embodiment, the resection guide 1020 includes one or more bone attachment features 1024. The bone attachment features 1024 are configured to couple the resection guide 1020 to a bone. In one embodiment, the bone attachment features 1024 are implemented as fasteners 1010 that can be inserted through the resection guide 1020 and into a bone (e.g., tibia 226). Alternatively, or in addition, the bone attachment features 1024 may include openings or holes through the resection guide 1020 that are sized to accept fasteners 1010. In one embodiment, the openings or holes have a greater diameter than a width of the resection feature 1022. In the illustrated embodiment, the openings or holes of the bone attachment features 1024 are positioned at the ends of the resection feature 1022.

The resection guide 1020 can be coupled to, or integrated with, or connected to, a fixation guide 990 such as fixation guide 1090. The fixation guide 1090 facilitates positioning, orientation, alignment, and/or deployment of one or more fixation members, including fasteners such as fasteners 1010 (e.g., K-wires or pins, also referred to as anchor pins). Certain fasteners, such as K-wires (of various diameters, such as between about 1.6 mm-2.0 mm) can be used as guides, also referred to as guide pins to facilitate deployment of other fasteners 1010 such as permanent or temporary bone screws (headless or headed bone screws of between about 4.0-4.5 mm in diameter). The bone screws may be cannulated and may be driven along the guide pins to a desired deployment location. The fixation guide 1090 can provide a particular trajectory, orientation, or direction and positioning for the guide pins and any bone screws that will be used.

Advantageously, in embodiments with the fixation guide 1090 connected to, or integrated with, the resection guide 1020, positioning and placement of the resection guide 1020 can automatically position and place the fixation guide 1090. In this manner, the location and orientation of the fixation guide 1090 is determined based on the placement of the resection guide 1020 because of a body, or coupler, that connects or couples the fixation guide 1090 and resection guide 1020.

In certain embodiments, the fixation guide 1090 includes one or more openings 1092 such as tubes, holes, or channels that extend from one side of the fixation guide 1090 to an opposite side of the fixation guide 1090. In one embodiment, the openings 1092 indicate a path for fasteners 1010 into a bone. For example, where the fixation guide 1090 is used to guide fasteners 1010 into a malleolus of a tibia 226, the openings 1092 may indicate a path for the fasteners 1010 through a distal fragment (e.g., the malleolus) and into a proximal fragment (e.g., the tibia 226).

These openings 1092 can provide a guide, path, trajectory, or the like for deployment of one or more fasteners 1010 through the openings 1092 and into a bone such as a long bone. Advantageously, the openings 1092 can be used by a surgeon to visualize an angle of entry for a fastener 1010 deployed into a long bone. Prior to deploying the fasteners 1010, a surgeon may visually check the angle of entry with the fixation guide 1090 positioned on or near the long bone. In certain cases, a surgeon may deploy a single fastener 1010 into bone by way of the openings 1092 (either with or without a deployed sleeve 1094) and then use fluoroscopy to see where this probe/targeting/test fastener 1010 is within the long bone. Based on these checks, a surgeon may confirm that the planned fixation guide 1090 and/or openings 1092 and/or sleeves 1094 are suitable and appropriate. Alternatively, based on these checks, a surgeon may decide to use a differently configured fixation guide 1090, opening 1092, and/or sleeve 1094. In one embodiment, the fastener 1010 deployed using the fixation guide 1090 may be temporary fasteners that provide a guide and/or one or more pilot holes for permanent fasteners that will be deployed later (e.g., cannulated bone screws, for example).

In certain embodiments, the fixation guide 1090 includes one or more sleeves 1094. Each sleeve 1094 may be configured to accept a fastener 1010. The sleeves 1094 may serve to ensure fasteners 1010 of a certain diameter are used. Alternatively, or in addition, sleeves 1094 can be used to deploy a fastener 1010 that is not coaxial with an opening 1092.

In the illustrated embodiment, the openings 1092 are each configured to accept and hold a sleeve 1094a,b. The sleeves 1094 may engage the holes by way of a friction fit. Alternatively, the openings 1092 may include threads that engage external threads of the sleeve 1094 to connect to and hold the sleeve 1094 in place. In one embodiment, the sleeves 1094 fit within the openings 1092 and guide deployment of the fasteners 1010 and/or a variety of other fasteners such as bone screws, or the like.

In the illustrated embodiment, the sleeves 1094 extend through the fixation guide 1090 and contact bone when the fixation guide 1090 is deployed. In certain embodiments, a distal end of the sleeves 1094 may be angled such that a distal end of the sleeve 1094 rests flush against a bone surface. A proximal end of the sleeves 1094 may include one or more projections that can aide a user such as a surgeon in deploying or removing the sleeve 1094 from the openings 1092. In one embodiment, a diameter an opening 1092 and a diameter of a corresponding sleeve 1094 are such that the interface between the opening 1092 and the sleeve 1094 can be one or more of a friction fit, press fit, interference fit, or slip fit. Advantageously, in one embodiment, the sleeve 1094 are interchangeable with each other and with other sleeves 1094. So, the osteotomy system 1000 can include a variety of sleeves 1094 each having the same or different internal diameters for working with different diameter fasteners.

Alternatively, or in addition, each sleeve 1094 may be of a specific diameter and only one of the openings 1092 may have a corresponding diameter for the opening such that each sleeve 1094 is configured to be used in a particular opening 1092. Having specific size openings 1092 and specific size sleeve 1094 can be useful if each sleeve is configured to accept a specific diameter fastener 1010. In this manner, the fixation guide 1090 can assist in desired deployment of fasteners 1010 of particular types and/or diameters. In one embodiment, a sleeve 1094 may be patient-specific. Alternatively, or in addition, a sleeve 1094 may be patient-matched.

In one embodiment, the fixation guide 1090 is patient-specific. As one example, the fixation guide 1090 so aspects of the fixation guide 1090 may be defined, determined, positioned on, or in, the body 1032 and/or have an orientation based on patient imaging data. The patient imaging data can be used to position and orient aspects of the fixation guide 1090 (e.g., openings 1092). In one example, as described in the present disclosure, patient imaging data can be used to generate bone models of bones of the patient. The bone models can be used to determine and/or define position, alignment, and/or trajectories for the openings 1092 and/or sleeves 1094 and/or number, position, and/or configuration of landmark registration features 1028, as well as other features and attributes of the fixation guide 1090.

Alternatively, or in addition, aspects of the fixation guide 1090 may be patient-matched. In certain embodiments, this may mean that the osteotomy system 1000 may include a plurality of fixation guide 1090, each with a differently configured trajectory, size, position, alignment and/or orientation of one or more openings 1092 and/or sleeves 1094. The differently configured aspects may be embodied in separate fixation guides 1090. The separate fixation guides 1090 may be connected to separate resection guides 1020. Alternatively, or in addition, these separate fixation guides 1090 may be configured to couple to a resection guide 1020 by way of a coupler 1096 as described herein.

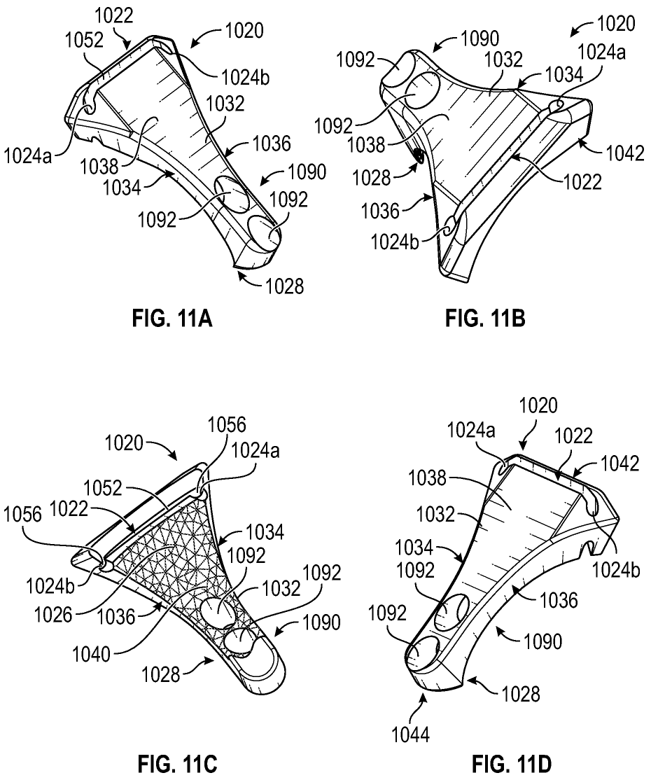

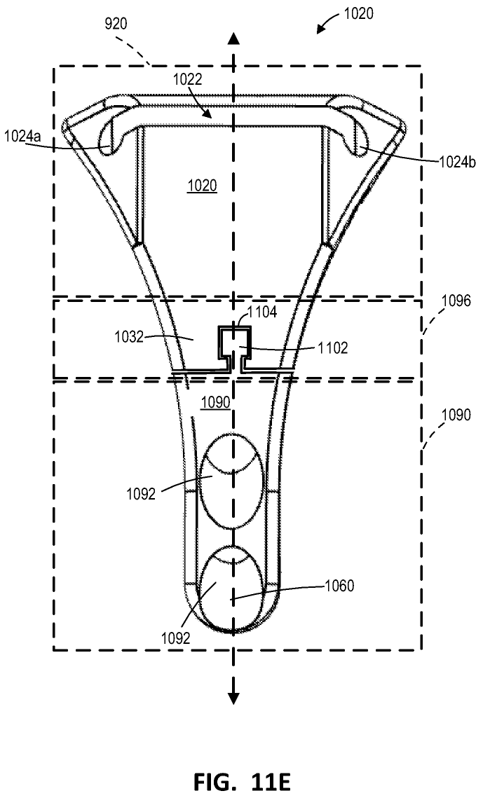

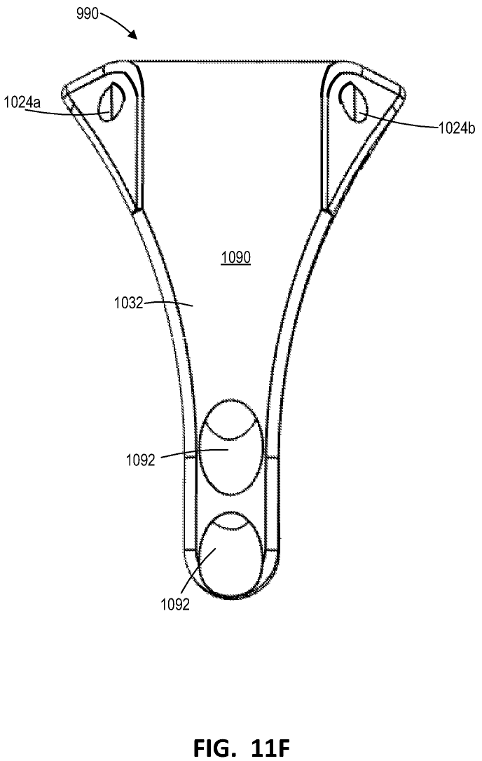

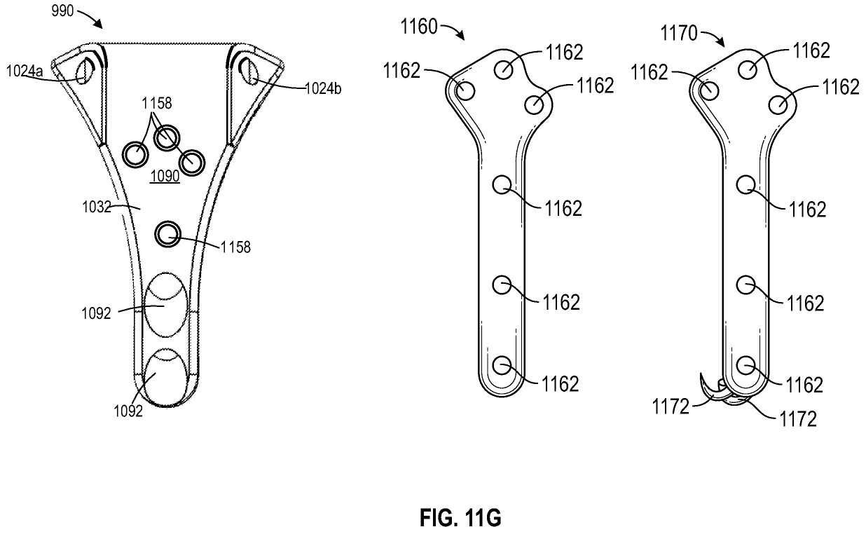

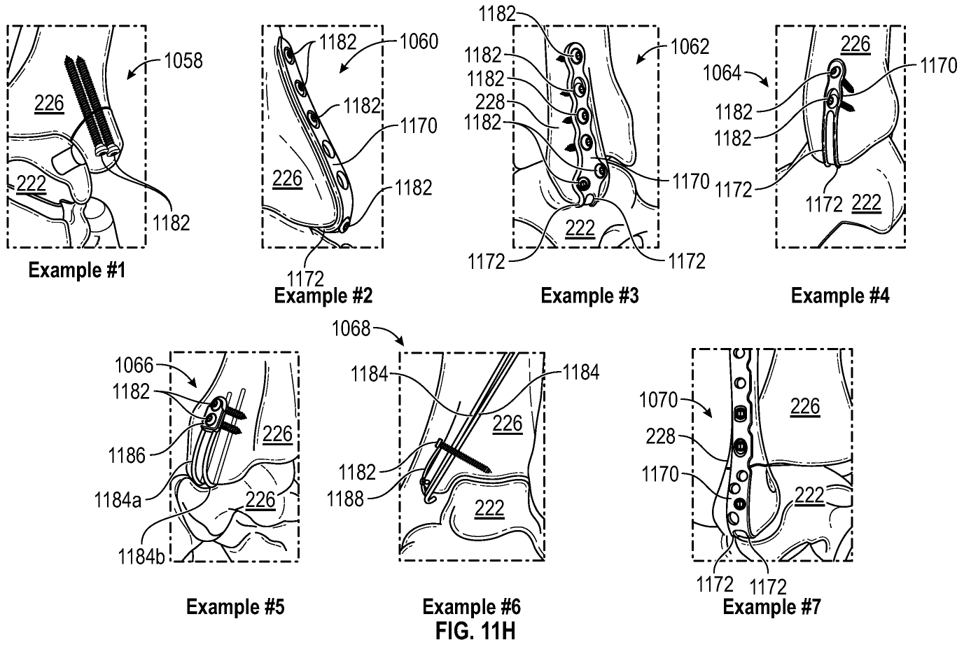

FIGS. 11A-11D illustrate views of an example resection guide 1020 of the system 1000 of FIG. 10, according to one embodiment. The resection guide 1020 may include a single resection feature 1022, one or more bone attachment features 1024, and/or may include one or more bone engagement surfaces 1026. In certain embodiments, the resection guide 1020 may include one or more landmark registration features 1028. Other embodiments may not include landmark registration features 1028. The resection guide 1020 may include a body 1032 that supports the resection feature 1022, one or more bone attachment features 1024, and/or one or more bone engagement surfaces 1026.

The body 1032 may include a resection feature 1022 that guides a cutting tool to resect one or more bones, such as a distal end of a tibia in the manner needed to make the desired resection. For example, the resection features 1022 may be used to guide a planar cutting blade, an arcuate cutting blade, a drill or mill, a burr, and/or the like. In one example, the resection feature 1022 can guide resection of bone to separate a malleolus from the bone. The resection features 1022 may guide a reciprocating planar blade, such as that of a surgical bone saw, which forms planar cuts in a distal tibia. Various manual or powered tools may be used to form the cuts. In one embodiment, a sagittal bone saw can be used. In one example, the resection feature 1022 may take the form of a single slot 1052 in a straight line.

The body 1032 may include a pattern (not shown) of openings that extend from one surface or side to an opposite surface or side. In one embodiment, the pattern is a honeycomb pattern of holes in the shape of hexagons. The pattern can serve to enhance visualization by a surgeon of bone contacting the resection guide 1020. In addition, the pattern can reduce the amount of material needed to fabricate the resection guide 1020. In certain embodiments, the resection guide 1020 may be fabricated from metal using additive manufacturing. Furthermore, the pattern can provide an aesthetic benefit for the resection guide 1020/fixation guide 1090. In another embodiment, the body 1032 includes a single opening and/or a plurality of openings that enhance visualization by a surgeon of bone near the resection guide 1020.

The resection guide 1020/fixation guide 1090 includes an anterior side 1034, a posterior side 1036, a medial side 1038, a lateral side 1040, a superior side 1042, and an inferior side 1044. FIG. 11A is an inferior anterior perspective view. FIG. 11B is a superior posterior perspective view. FIG. 11C is bone contact side perspective view. FIG. 11D is a medial posterior perspective view.

In one embodiment, the resection guide 1020 may include an alignment guide (not shown). The alignment guide may be a part of, connected to, or extend from the body 1032 or may be a separate component. The alignment guide can serve as a guide to orient the resection guide 1020 relative to a longitudinal axis of a bone (e.g., a long axis of a tibia 226). The alignment guide can include a structure that extends and includes a hole sized to accept and/or retain a fastener 1010 such as a K-wire. The hole can be positioned such that with a K-wire in the hole, a surgeon can align the K-wire with a longitudinal axis of a bone, such as a tibia, to ensure that the features of the resection guide 1020/fixation guide 1090 are aligned relative to the longitudinal axis of the bone. The alignment guide may assist a surgeon in desired placement and orientation of the resection guide 1020 during a surgical procedure.

In one embodiment, a single slot 1052 extends from the medial side 1038 to the lateral side 1040. The single slot 1052 may also extend from near the anterior side 1034 to near the posterior side 1036 and may be positioned near the superior side 1042. In the illustrated embodiment, the single slot 1052 is straight. Of course, the single slot 1052 can be curved.

The bone engagement surface 1026 facilitates and assists a surgeon in locating a desired position for the resection guide 1020 and/or fixation guide 1090 intraoperatively. Those of skill in the art will appreciate that the resection guide 1020 and/or the fixation guide 1090 can each separately or together include one or more bone engagement surfaces 1026. Alternatively, or in addition, a bone engagement surface 1026 on one of the resection guide 1020 or the fixation guide 1090 can have a different level of detail and/or fidelity than the other bone engagement surface 1026. This difference in fidelity can be helpful for bone surfaces that are smoother, like a medial surface of a distal tibia 226.

In one embodiment, the resection guide 1020 may include a bone engagement surface 1026 configured to contact or seat on exposed bone of the patient and the fixation guide 1090 may include a bone engagement surface 1026 configured to contact or seat on exposed bone of the patient. Alternatively, or in addition, the resection guide 1020 may include a bone engagement surface 1026 configured to contact or seat on exposed bone of the patient and the fixation guide 1090 may include a skin engagement surface configured to contact or seat on the surface of skin of the patient. Alternatively, or in addition, the fixation guide 1090 may include a bone engagement surface 1026 configured to contact or seat on exposed bone of the patient and the resection guide 1020 may include a skin engagement surface configured to contact or seat on the surface of skin of the patient. Using one of these embodiments, a user may be able to resect the bone using one or more minimally invasive surgical techniques.

In one embodiment, the bone engagement surface 1026 may be defined based on medical imaging of one or more bones of a patient and/or anatomic data of a patient. Alternatively, or in addition, the bone engagement surface 1026 may be defined based on one or more bone models that may be based on medical imaging of one or more bones of a patient. For example, a bone model of a tibia 226 (or at least a distal end of the tibia 226) of the patient may be used to create a bone engagement surface 1026 that is a mirror image of a surface of the medial surface of the tibia 226. In certain embodiments, the bone engagement surface 1026 may be formed using a medial surface and/or part of an anterior surface and part of a posterior surface of a bone model of a tibia 226 of a patient.

In certain embodiments, the resection guide 1020 and/or the fixation guide 1090 may include one or more landmark registration features 1028. The landmark registration features 1028 can be configured to engage with specific surface features (e.g., landmarks) of bone or skin of a patient and guide a surgeon in positioning the resection guide 1020 and/or fixation guide 1090 in a desired positioning on or relative a bone of a patient.

Upon desired positioning of the resection guide 1020 (using one or more of a bone engagement surface 1026, an alignment guide, and/or one or more landmark registration features 1028) a surgeon may activate or deploy two or more of the bone attachment features 1024 by deploying fasteners 1010 in holes or openings of the bone attachment features 1024 to secure the resection guide 1020 in place on the bone. The positioning of the resection guide 1020 places the single slot 1052 in the desired position for resecting a malleolus during a surgical osteotomy for correcting a condition.

In alternative embodiments, a resection feature may be designed to guide a different type of cutter, such as a drill, mill, or side-cutting burr. In such embodiments, the resection feature may not be a slot, but may instead be a translatable or rotatable cutter retainer that guides translation and/or rotation of the cutter relative to the bone. A resection feature embodied as a slot is one example of a resection feature.

Alternatively, or in addition, in certain embodiments, one or more of resection features 1022 may be positioned on, or in, the body 1032 and/or have an orientation based on patient imaging data (e.g., patient-specific). The patient imaging data can be used to position and orient the resection feature 1022 resection and subsequent retraction of the malleolus enables a remediation procedure on a bone such as a talus, and superior and anterior parts of the talus. For example, as described in the present disclosure, patient imaging data can be used to generate bone models of bones of the patient. The bone models can be used to determine and/or define contours for a bone engagement surface 1026, a position for a single slot 1052, an orientation for the single slot 1052, as well as other features and attributes of one or more patient specific instruments that can be used in a procedure.

Alternatively, or in addition, the position and/or orientation/trajectory of the resection feature 1022 may be patient-matched. In certain embodiments, this may mean that the osteotomy system 1000 may include a plurality of resection guides 1020, each with a differently configured resection feature 1022. The differently configured resection feature 1022 may each be designed to accommodate a particular set of patients and/or patients presenting with a particular set of conditions and/or anatomical characteristics. In such embodiments, the resection guide 1020 may, or may not, include one or more bone engagement surfaces 1026.

The resection guide 1020 includes one or more bone attachment features 1024. As embodied in FIGS. 11A through 11E, the bone attachment features 1024 may take the form of one or more holes 1056 that cooperate with fasteners 1010 and extend from the medial side 1038 to the lateral side 1040. The holes 1056 may be shaped to accommodate pins, K-wires, and/or other elongated bone fixation elements that can be anchored in a bone to keep the resection guide 1020 in place. In addition, the holes 1056 may be angled through the body 1032 to match a trajectory of the resection feature 1022.

In the illustrated embodiment, the resection guide 1020 and/or fixation guide 1090 include an anterior bone attachment feature 1024a and a posterior bone attachment feature 1024b. In one embodiment, a hole 1056 of the anterior bone attachment feature 1024a aligns with a posterior bone attachment feature 1024b. Alternatively, or in addition, the anterior bone attachment feature 1024a and posterior bone attachment feature 1024b are not aligned with each other.

Alternatively, or in addition, the anterior bone attachment feature 1024a and posterior bone attachment feature 1024b align with a resection feature 1022. Alignment of the bone attachment features 1024 with each other and/or with a resection feature 1022 and/or with a trajectory for the resection feature 1022 can assist a surgeon in preparing for an osteotomy of the tibia 226. For example, this alignment can be used to visualize where the osteotomy will be made in the bone, because fastener 1010 deployed in the bone attachment features 1024 can be seen on fluoroscopy and extend into the bone at a trajectory that matches the resection feature 1022. Thus, with the fasteners 1010 deployed into the bone, a surgeon can see where the osteotomy formed using the resection feature 1022 will go, prior to performing the osteotomy.

In the illustrated embodiment, the resection guide 1020 is integrated with a fixation guide 1090. The fixation guide 1090 can serve multiple functions. In one embodiment, the fixation guide 1090 facilitates deployment of fasteners 1010 for use to identify a position for a bone fragment before it is separated from the bone. In this manner, a surgeon may perform a type of preemptive fixation, meaning steps are taken prior to resection that will facilitate fixation after the resection.

For example, before resecting a malleolus, a surgeon may deploy fasteners 1010 through one or more sleeves 1094 deployed in one or more openings 1092. The fasteners 1010 may be deployed from a medial surface of the malleolus and extend into the tibia and across a plane that will be formed when a cutting tool cuts the bone as guided by a resection feature 1022. Next, a surgeon may remove the fasteners 1010 leaving holes, channels, bone tunnels, in the bone where the fasteners 1010 were. Next, a bone portion such as a malleolus may be resected from the tibia (e.g., using the resection feature 1022). When the bone fragment, (e.g., malleolus), is reduced to its original location against the tibia, a surgeon may redeploy the fasteners 1010 using the same holes/bone tunnels formed when the fasteners 1010 were first deployed. The fasteners 1010 may be deployed in the preformed bone tunnels of the bone fragment (e.g., malleolus) and corresponding bone tunnels preformed in the tibia. In this manner, the fasteners 1010 are deployed in the exact location of bone segments on either side of a cut that will be made when the resection feature 1022 is used to cut the bone. This technique enables and assures a proper reduction and union of the resected bone fragment with its corresponding bone portion.

Advantageously, the fixation guide 1090 includes openings 1092 and/or sleeves 1094 that are positioned, sized, oriented, and configured according to surgeon preferences, conventional techniques, patient specific anatomy, and the like. Those of skill in the art will appreciate that the fixation guide 1090 may include more or fewer than two openings 1092 and the openings 1092 can be aligned vertically or horizontally. In the illustrated embodiment, one or more of the openings 1092 are aligned vertically such that when the resection guide 1020 and fixation guide 1090 are used together the one or more of the openings 1092 align with a landmark. In the illustrated embodiment, the landmark is a longitudinal axis 1060. The longitudinal axis 1060 may be a longitudinal axis 1060 of a tibia 226 of a user/patient. Of course, any landmark may be used as a references for the positioning of the openings 1092. In certain embodiments, the sleeves 1094 may be permanently connected to the fixation guide 1090. Alternatively, as in the illustrated embodiment, the sleeves 1094 may removably engage with the fixation guide 1090.

FIGS. 11A-11D illustrate an embodiment of a resection guide 1020 coupled to a fixation guide 1090 by way of a solid body 1032. FIG. 11E illustrates an alternative embodiment in which the body 1032 includes a coupler 1096 that couples a resection guide 1020 to a fixation guide 1090.

The coupler 1096 may enable the resection guide 1020 and fixation guide 1090 to be coupled or decoupled preoperatively or intraoperatively. The coupler 1096 may position the fixation guide 1090 relative to the resection guide 1020. Said another way, engaging the fixation guide 1090 to the resection guide 1020 by way of the coupler 1096 may position the fixation guide 1090 in a desired position and/or orientation (e.g., a position and/or orientation predetermined in a preoperative plan). In one embodiment, use of the coupler 1096 can ensure that a location and/or an orientation of one or more openings of a fixation guide 1090 are determined based on the resection guide 1020 Similarly, the coupler 1096 can ensure that a location and/or an orientation of one or more features of the resection guide 1020 (e.g., resection features 1022) are determined based on a position and/or orientation of the fixation guide 1090.

Advantageously, a coupler 1096 may enable coupling of a single resection guide 1020 to a plurality of different fixation guides 1090. Each of the plurality of different fixation guides 1090 may include different configurations of openings 1092, different sizes of openings 1092, openings 1092 that accept sleeves 1094, openings 1092 of different orientations, angles, trajectories, or the like.

Similarly, the coupler 1096 may enable coupling of a single fixation guide 1090 to a plurality of different resection guide 1020, including no resection guide 1020 or a structure that does not include a resection feature 1022. Each of the plurality of different resection guides 1020 may include different configurations of bone attachment features 1024, different sizes and configurations of resection features 1022 , one or more resection features 1022 of different orientations, angles, or the like.

Referring now to FIG. 11E, the coupler 1096 may include a key 1102 and a slot 1104. The slot 1104 may be shaped to accept the key 1102 when the fixation guide 1090 is aligned with the resection guide 1020. Advantageously, where an osteotomy system 1000 includes a plurality of resection guides 1020 and/or a plurality of fixation guides 1090, certain resection guides 1020 may include a slot 1104 configured to only accept fixation guides 1090 having a particular key 1102 and certain fixation guide 1090 may include a key 1102 configured to fit into particular slot 1104 of certain resection guide 1020. Those of skill in the art will appreciate that the resection guide 1020 can include the key 1102 and the fixation guide 1090 can include the slot 1104. In this manner, the osteotomy system 1000 can facilitate ensuring that a correct or desired resection guide 1020 is paired with a similarly desired and correct fixation guide 1090, and vice versa.

FIG. 11F illustrates an alternative embodiment for an osteotomy system. Specifically, FIG. 11F illustrates an alternative embodiment of a fixation guide 1090 for use with an osteotomy system. The fixation guide 1090 may have many structures, features, and functions, operations, and configuration similar or identical to those of embodiments described in relation to FIGS. 10-11E, like parts are identified with the same reference numerals. Accordingly, the fixation guide 1090 may include a bone attachment feature 1024a, a bone attachment feature 1024b, a body 1032, and/or one or more openings 1092.

Notably absent from the embodiment of a fixation guide 1090 of FIG. 11F is a resection feature 1022. The fixation guide 1090 can be used to position and/or orient one or more fasteners 1010 (not shown in FIG. 11F) such as pins or K-wires. The openings 1092 can be sized to receive sleeves 1094 and/or the openings 1092 can be sized to receive the fasteners 1010. The openings 1092 can indicate a path for fasteners into a long bone (e.g., tibia 226, fibula 228, a femur, or the like).

FIG. 11G illustrates a guide according to one embodiment and example fixation hardware that can be used with the guide. Often, orthopedic surgical procedure include deployment of temporary and/or permanent fixation (e.g., fasteners, aka fixation hardware). Often, deployment of the fixation is done by forming holes in one or more bones of a patient before the fasteners are deployed. The holes in the bones can serve as guides for the subsequently deployed fasteners. The holes in the bones can/may serve as ‘pilot’ holes and can be formed by a drill and drill bit, by a burr, and/or by deploying a pin such as a k-wire that leaves a bone tunnel or hole when removed. In certain embodiments, a surgeon may have a predetermined location for the fixation hardware. Taking the steps of positioning the fixation hardware and forming these pilot holes can add time, expense, and stress to a surgeon performing the surgical procedure.

Advantageously, the present disclosure includes a resection guide 1020 and/or a fixation guide 1090 that is designed, fabricated, and/or engineered to facilitate a surgeon forming pilot holes in bone(s) of a patient for a single set of fixation hardware and/or a plurality of different sets of fixation hardware.

The fixation guide 990 includes a plurality of openings/holes positioned, configured, and/or oriented for formation of one or more holes 1158 for use with fixation hardware. In certain embodiments, the holes 1158 are separate independent holes. In other embodiments, the holes 1158 can include the same holes formed in bone(s) by fasteners deployed using sleeves 1094 that fit in openings 1092.

FIG. 11G includes one example of an embodiment of the present disclosure, a fixation guide 990 that can enable formation of one or more pilot holes for subsequently deployed fixation hardware and two examples of suitable fixation hardware (e.g., bone plates) that can utilize the pilot holes formed using the fixation guide 990. Those of skill in the art will appreciate that the fixation guide 990 is but one example of a device that can be used to provide pilot holes according to embodiments of the present disclosure. In certain embodiments, other embodiments such as the resection guide 1020 and fixation guide 1090 of FIG. 11E can also be used and can include holes 1158 configured to facilitate forming pilot holes in bone(s) or bone fragments. For one set of fixation hardware certain holes 1158 may be used and for other fixation hardware certain holes 1158 may not be used. Of course, a surgeon may opt to not use any of the holes 1158. Advantageously in certain embodiments, the embodiments of the present disclosure can function as a template for forming pilot holes or other structures that extend into or extend out from bone(s) for use with one or more sets of fixation hardware.

FIG. 11G illustrates a bone plate 1160 and a hook plate 1170. The bone plate 1160 is one example of fixation hardware that can make use of pilot holes formed in bone(s) of a patient using embodiments of the present disclosure. The bone plate 1160 can include a plurality of holes 1162 or openings that may be sized and/or configured to accept bone screws, headless bone screws, compression screws, or the like. Alternatively, or in addition, the holes 1162 may be holes that include a countersunk top portion to accept a bone screw, compression holes, or the like. Advantageously, the holes 1162 of the bone plate 1160 are positioned an align with pilot holes in bone(s) formed using the holes 1158 of the fixation guide 990/fixation guide 1090.

The hook plate 1170 is another example of fixation hardware that can make use of pilot holes formed in bone(s) of a patient using embodiments of the present disclosure. The hook plate 1170 can include a plurality of holes 1162 or openings that may be sized and/or configured to accept bone screws, headless bone screws, compression screws, or the like. Alternatively, or in addition, the holes 1162 may be holes that include a countersunk top portion to accept a bone screw, compression holes, or the like. Advantageously, the holes 1162 of the hook plate 1170 are positioned an align with pilot holes in bone(s) formed using the holes 1158 of the fixation guide 990/fixation guide 1090.

In addition, the hook plate 1170 can include one or more hooks 1172 that may extend from a distal end of the hook plate 1170. In one embodiment, the one or more hooks 1172 are configured to extend around a distal end and surface of a medial malleolus (or a lateral malleolus) and thereby engage a bone portion of the medial malleolus (or a lateral malleolus). Alternatively, or in addition, the one or more hooks 1172 are configured to engage holes in or near a distal end of a medial malleolus (or a lateral malleolus) and thereby engage a bone portion of the medial malleolus (or a lateral malleolus). Advantageously, certain embodiments of the present disclosure can also include holes 1158 positioned, aligned, oriented and configured to a surgeon to use those holes 1158 to form openings that can accept the one or more hooks 1172 of a hook plate 1170. In another embodiment, the one or more hooks 1172 are configured (e.g., include pointed ends)

Of course, bone plates such as bone plate 1160 and hook plates such as hook plate 1170 are not the only types of fixation hardware. The present disclosure can be used to prepare for deployment of the fixation hardware. Those of skill in the art will appreciate that there are a variety of different kinds and/or types of fasteners, fixation devices, and/or fixation hardware that can benefit from pilot holes or other structures that embodiments of the present disclosure can provide.

FIG. 11H illustrates a variety of example fixation hardware (e.g., systems, devices, and the like) that can be used with embodiments of guides and/or systems of the present disclosure.

Example #1 illustrates a fixation system 1058 that includes a pair of bone screws 1182. Advantageously, the bone screws 1182 can be deployed into holes formed using fasteners a surgeon has already deployed (or previously deployed and removed) using the fixation guide 1090 of the present disclosure. The fixation guide 1090 enables a surgeon to deploy the bone screws 1182 with the medial malleolus bone fragment reduced in substantially its same original position before the osteotomy. The bone screws 1182 may include heads or may be headless.

Example #2 illustrates a fixation system 1060 that includes hook plate 1170 and a plurality of bone screws 1182. Advantageously, the bone screws 1182 can be deployed into holes formed using the fixation guide 1090 and/or resection guide 1020 of the present disclosure as a template. The fixation guide 1090 and/or resection guide 1020 enables a surgeon to deploy the bone screws 1182 with the medial malleolus bone fragment reduced and/or compressed. The hook plate 1170 includes one or more hooks 1172 that are inserted into a distal cortex of the medial malleolus (either pressed or tapped into bone without holes and/or inserted into holes formed using holes 1158 of embodiments of the fixation guide 1090 and/or resection guide 1020.

Example #3 illustrates a fixation system 1062 that includes hook plate 1170 and a plurality of bone screws 1182. In Example #3, the fixation system 1088 is deployed on a fibula 228. Advantageously, the bone screws 1182 can be deployed into holes formed using the fixation guide 1090 and/or resection guide 1020 of the present disclosure as a template. The fixation guide 1090 and/or resection guide 1020 enables a surgeon to deploy the bone screws 1182 with the lateral malleolus bone fragment reduced and/or compressed. The hook plate 1170 includes one or more hooks 1172 that are inserted into a distal cortex of the lateral malleolus (either pressed or tapped into bone without holes and/or inserted into holes formed using holes 1158 of embodiments of the fixation guide 1090 and/or resection guide 1020.

Example #4 illustrates a fixation system 1064 that includes hook plate 1170 and a plurality of bone screws 1182. Advantageously, the bone screws 1182 can be deployed into holes formed using the fixation guide 1090 and/or resection guide 1020 of the present disclosure as a template. The fixation guide 1090 and/or resection guide 1020 enables a surgeon to deploy the bone screws 1182 with the medial malleolus bone fragment reduced and/or compressed. The hook plate 1170 includes one or more elongated hooks 1172 that are inserted into a distal cortex of the medial malleolus (either pressed or tapped into bone without holes and/or inserted into holes formed using holes 1158 of embodiments of the fixation guide 1090 and/or resection guide 1020.

Example #5 illustrates a fixation system 1066 that includes wire 1184, a body 1186, and a plurality of bone screws 1182. Advantageously, the bone screws 1182 can be deployed into holes formed using the fixation guide 1090 and/or resection guide 1020 of the present disclosure as a template. Alternatively, or in addition, holes for the wire 1184 on a distal end of the medial malleolus can be formed using holes in the fixation guide 1090 and/or resection guide 1020 of the present disclosure as a template. The wire 1184 may originate within a hole or bone tunnel, exit the bone, loop around the body 1186, reenter the bone by way of a bone tunnel or hole and terminate within the bone. Advantageously, the bone tunnels can be formed using the fixation guide 1090 and/or resection guide 1020 of the present disclosure as a template.

Example #6 illustrates a fixation system 1068 that includes a plurality of wires 1184 (each wire may have a hook on a distal end), one or more bone screws 1182, and a suture 1188. Advantageously, the bone screw(s) 1182 can be deployed into a hole formed using the fixation guide 1090 and/or resection guide 1020 of the present disclosure as a template (e.g., See FIG. 11G). Alternatively, or in addition, holes for the wires 1184 on a distal end of the medial malleolus can be formed using holes in the fixation guide 1090 and/or resection guide 1020 of the present disclosure as a template. The wires 1184 may extend into a medial malleolus bone fragment and into the tibia. A hook on a distal end of the wires 1184 may engage a distal cortex of the medial malleolus bone fragment or seat in a hole of the medial malleolus bone fragment. Advantageously, the bone tunnels can be formed using the fixation guide 1090 and/or resection guide 1020 of the present disclosure as a template.

Example #7 illustrates a fixation system 1070 that includes hook plate 1170 for a fibula and a plurality of bone screws 1182, which may have not yet been deployed. Advantageously, the bone screws 1182 can be deployed into holes formed using the fixation guide 1090 and/or resection guide 1020 of the present disclosure as a template. The fixation guide 1090 and/or resection guide 1020 enables a surgeon to deploy the bone screws 1182 with the lateral malleolus bone fragment reduced and/or compressed. The hook plate 1170 may include one or more hooks 1172 that are inserted into a distal cortex of the medial malleolus (either pressed or tapped into bone without holes and/or inserted into holes formed using holes 1158 of embodiments of the fixation guide 1090 and/or resection guide 1020.

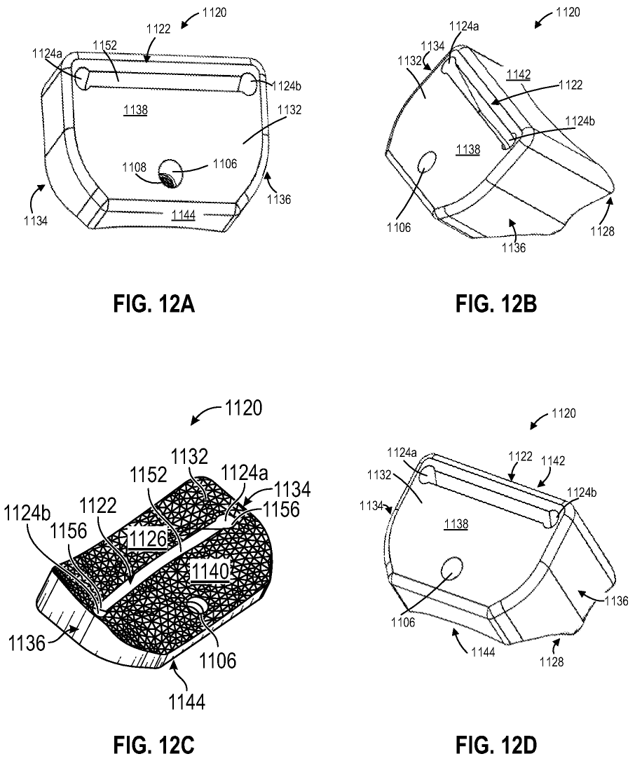

FIGS. 12A-12D illustrate views of an example resection guide 1120 of the system 1000 of FIG. 10, according to one embodiment. The example resection guide 1120 may have many structures, features, and functions, operations, and configuration similar or identical to those of embodiments described in relation to FIGS. 10-11E, like parts are identified with the same reference numerals. The description of the features and aspects of these like parts identified with the same reference numerals included elsewhere in the present disclosure can be applied to the embodiments of FIGS. 12A-12D, and vice versa.

The resection guide 1120 includes a single resection feature 1122, one or more bone attachment features 1124, and/or may include one or more bone engagement surfaces 1126. In certain embodiments, the resection guide 1120 may include one or more landmark registration features 1128. Other embodiments may not include landmark registration features 1128. The resection guide 1120 may include a body 1132 that supports the resection feature 1122, one or more bone attachment features 1124, and/or one or more bone engagement surfaces 1126.

The body 1132 may include a resection feature 1122 that guides a cutting tool to resect one or more bones, such as a distal tibia in the manner needed to make the desired resection. In certain embodiments, the resection feature 1122 guides resection of a long bone to separate a malleolus from the long bone. In one example, the resection features 1122 may be used to guide a planar cutting blade, an arcuate cutting blade, a drill or mill, a burr, and/or the like. In one example, the resection feature 1122 can guide resection of bone to separate a malleolus from the bone. The resection features 1122 may guide a reciprocating planar blade, such as that of a surgical bone saw, which forms planar cuts in a distal tibia. Various manual or powered tools may be used to form the planar cuts. In one embodiment, a sagittal bone saw can be used. In one example, the resection feature 1122 may take the form of a single slot 1152 in a straight line.

In one embodiment, the resection feature 1122 includes an opening (e.g., single slot 1152) that extends from a bone-facing side (e.g., lateral side 1140) to a side opposite the bone-facing side. As described herein, a bone-facing side is the side of a device that faces bone when the device is in use. In the illustrated embodiment, the opening is an elongated opening.

The body 1132 may include a pattern (not shown) of openings or a single opening or a combination of openings that extend from one surface or side to an opposite surface or side. In one embodiment, the pattern is a honeycomb pattern of holes in the shape of hexagons. The pattern can serve to enhance visualization by a surgeon of bone contacting the resection guide 1120. In addition, the pattern can reduce the amount of material needed to fabricate the resection guide 1120. In certain embodiments, the resection guide 1120 may be fabricated from metal using additive manufacturing. Furthermore, the pattern can provide an aesthetic benefit for the resection guide 1020/fixation guide 1190.

The resection guide 1120 includes a body 1132 that includes an anterior side 1134, a posterior side 1136, a medial side 1138, a lateral side 1140, a superior side 1142, and an inferior side 1144. FIG. 12A is an inferior anterior perspective view. FIG. 12B is a superior posterior perspective view. FIG. 12C is bone contact side perspective view. FIG. 12D is a medial posterior perspective view.

In one embodiment, the resection guide 1120 may include an alignment guide (not shown). The alignment guide may be a part of, connected to, or extend from the body 1132 or may be a separate component. The alignment guide can serve as a guide to orient the resection guide 1120 relative to a longitudinal axis of a bone. The alignment guide can include a structure that extends and includes a hole sized to accept and/or retain a fastener 1010 such as a K-wire. The hole can be positioned such that with a K-wire in the hole, a surgeon can align the K-wire with a longitudinal axis of a bone, such as a tibia, to ensure that the features of the resection guide 1120 are aligned or positioned relative to the longitudinal axis of the bone. The alignment guide may assist a surgeon in desired placement and orientation of the resection guide 1120 during a surgical procedure.

In one embodiment, a single slot 1152 extends from the medial side 1138 to the lateral side 1140. The single slot 1152 is one example of a resection feature 1122. The bone engagement surface 1126 facilitates a surgeon locating a desired position for the osteotomy system 1000 intraoperatively. Those of skill in the art will appreciate that the resection guide 1120 can include one or more bone engagement surfaces 1126.

In certain embodiments, the resection guide 1120 may include one or more landmark registration features 1128. The landmark registration features 1128 can be configured to engage with specific surface features of bone or skin of a patient guide a surgeon in positioning the resection guide 1120 in a desired positioning on or relative a bone of a patient.

Upon desired positioning of the resection guide 1120 (using one or more of a bone engagement surface 1126, an alignment guide, and/or one or more landmark registration features 1128) a surgeon may activate two or more of the bone attachment features 1124 by deploying fasteners 1010 in the bone attachment features 1124 to secure the resection guide 1120 in place on the bone. The positioning of the resection guide 1120 places the single slot 1152 in the desired position for resecting a malleolus during a surgical osteotomy for correcting a condition.

In alternative embodiments, a resection feature may be designed to guide a different type of cutter, such as a drill, mill, or side-cutting burr. In such embodiments, the resection feature may not be a slot, but may instead be a translatable or rotatable cutter retainer that guides translation and/or rotation of the cutter relative to the bone.

Alternatively, or in addition, in certain embodiments, one or more resection features 1122 may be positioned on, or in, the body 1132 and/or have an orientation based on patient imaging data. The patient imaging data can be used to position and orient the resection feature 1122 resection and subsequent retraction of the malleolus enables a remediation procedure on a bone such as a talus, and superior and anterior parts of the talus. For example, as described in the present disclosure, patient imaging data can be used to generate bone models of bones of the patient. The bone models can be used to determine and/or define contours for a bone engagement surface 1126, a position for a single slot 1152, an orientation for the single slot 1152, as well as other features and attributes of one or more patient specific instruments that can be used in a procedure.

In certain embodiments, one or more of bone engagement surfaces 1126 may be patient-specific. For example, the bone engagement surfaces 1126 on the lateral side 1140 may be defined based on patient imaging data (e.g., patient-specific). The patient imaging data can be used to define the contour of one or more surfaces of the lateral side 1140. For example, as described in the present disclosure, patient imaging data can be used to generate bone models of bones of the patient. The bone models can be used to determine and/or define contours for the bone engagement surface(s) 1026, as well as other features and attributes of one or more patient specific instruments that can be used in a procedure.

Alternatively, or in addition, the bone engagement surfaces 1126 may be patient-matched. In certain embodiments, this may mean that an osteotomy system may include a plurality of resection guides 1120, each with a differently configured bone engagement surface 1126, resection feature 1022, bone attachment feature 1124, or the like. A differently configured bone engagement surfaces 1126 may include more or less fidelity in the contour of the bone engagement surfaces 1126 among a plurality of resection guides 1120. Alternatively, or in addition, the differently configured resection feature 1122 may each be designed to accommodate a particular set of patients and/or patients presenting with a particular set of conditions and/or anatomical characteristics. In such embodiments, the resection guide 1120 may, or may not, include one or more bone engagement surfaces 1126.

In the illustrated embodiment, the bone engagement surface 1126 is on a bone-facing side of the body 1132. The bone engagement surface 1126 may be configured to engage a surface of a patient. The surface of the patient can be one of a variety of surfaces. For example, in one embodiment, the surface of the patient is a surface of a long bone, (e.g., a tibia 226, fibula 228, or the like). In such an embodiment, a bone engagement surface 1126 may be shaped to match a contour of the surface of the long bone. As described herein, the bone engagement surface 1126 of the resection guide 1120 can be defined based at least in part on medical imaging of a portion of the long bone.

In certain embodiments, the bone engagement surface 1126 of the resection guide 1120 can be defined based at least in part on a bone model. The bone model may be for a typical long bone. For example, the bone model may be generated based on a typical long bone for patients having a certain set of characteristics and/or attributes (e.g., the bone model may be patient-matched). Alternatively, or in addition, the bone model may be generated from medical imaging of a portion of a long bone. In certain embodiments, the bone model may be generated from medical imaging of a portion of a long bone of the patient receiving a surgical procedure.

In another example, the surface of the patient may be a surface of skin of a patient. In one embodiment, the surface of the patient is a surface of skin covering a long bone. In such embodiments, the resection guide 1120 can be used with an MIS surgical procedure. Initially an anterior-posterior incision may be made where an osteotomy is to be made and skin retracted. Next, the resection guide 1120 may be positioned on the skin such that the resection feature 1122 is aligned with the incision.

In another embodiment, the resection guide 1120 may be positioned on the skin and fasteners 1010 may be deployed through the holes 1156, through the skin (e.g., a puncture incision) and into a long bone. Next, a user may form an incision in the skin by moving a cutting tool within the resection feature 1122 (e.g., single slot 1152). The cutting of the skin may expose the long bone.

The resection guide 1120 includes one or more bone attachment features 1124. As embodied in FIGS. 12A through 12D, the bone attachment features 1124 may take the form of one or more holes 1156 that cooperate with fasteners 1010 and extend from the medial side 1138 to the lateral side 1140. The holes 1156 may be shaped to accommodate pins, K-wires, and/or other elongated bone fixation elements that can be anchored in a bone to keep the resection guide 1120 in place.

In the illustrated embodiment, the resection guide 1120 include an anterior bone attachment feature 1124a and a posterior bone attachment feature 1124b. In one embodiment, a hole 1156 of the anterior bone attachment feature 1124a aligns with a posterior bone attachment feature 1124b. Alternatively, or in addition, the anterior bone attachment feature 1124a and posterior bone attachment feature 1124b are not aligned with each other. Alternatively, or in addition, the anterior bone attachment feature 1124a and posterior bone attachment feature 1124b align with a resection feature 1122.

In the illustrated embodiment, the resection guide 1120 can include a handle engagement feature 1106. The handle engagement feature 1106 is configured to couple with a handle (not shown). The handle can be used by a surgeon to place, position, orient, move, and/or secure the resection guide 1120 in place during different stages of a surgical procedure. In the illustrated embodiment, the handle engagement feature 1106 is implemented as a hole that extends from the medial side 1138 (the surface) down into the body 1132. In certain embodiments, the hole of the handle engagement feature 1106 can extend from the medial side 1138 to the lateral side 1140.

In one embodiment, a part of the handle engages with the handle engagement feature 1106 by way of a friction fit within the handle engagement feature 1106 (e.g., a hole). Alternatively, or in addition, the handle engagement feature 1106 can include internal threads 1108 that engage with external threads of the handle to secure the handle to the body 1132.

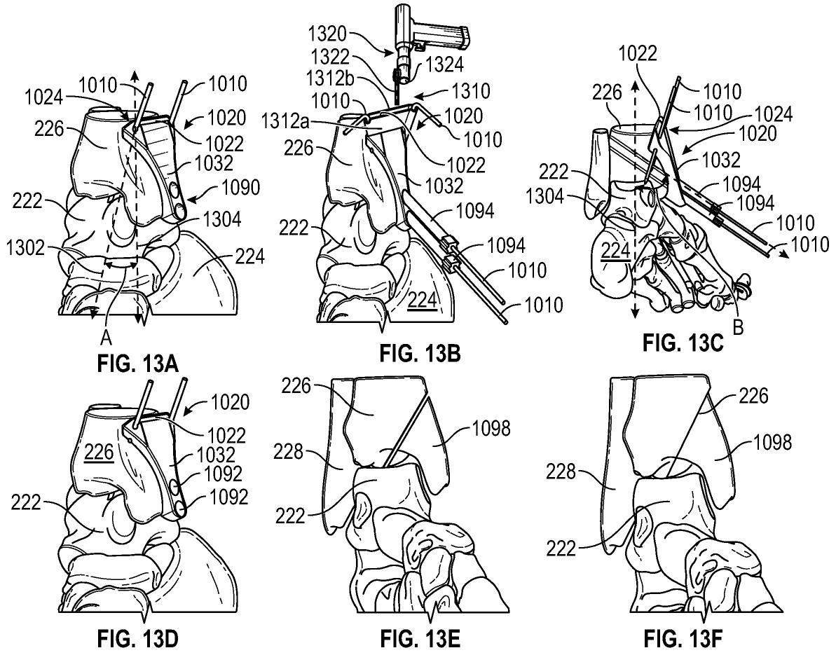

FIGS. 13A-13F illustrate different views of stages of a surgical procedure that uses the osteotomy system 1000. In FIG. 13A, a surgeon has positioned a resection guide 1020 that is combined with a fixation guide 1090. The surgeon has deployed fasteners 1010 into the holes 1056 to secure the combined resection guide 1020 and fixation guide 1090 to a distal tibia (i.e., the malleolus).

In one embodiment, the osteotomy system 1000 includes a combined resection guide 1020 and fixation guide 1090 that is patient-specific. Alternatively, or in addition, the combined resection guide 1020 and fixation guide 1090 is patient-matched. The patient-specific and/or patient-matched aspect of the combined resection guide 1020 and fixation guide 1090 may be reflected in an angle that the resection feature 1022 extends into the body 1032. In the illustrated embodiment, this angle, identified as angle A, may be measured between a trajectory 1302 of the resection feature 1022 and a long axis 1304 of the tibia 226.

In one embodiment, the resection feature 1022 is an opening in the body 1032 that serves as the resection feature 1022. The opening extends through the body 1032 from a medial side 1038 or superior side 1042 to a lateral side 1040. The angle A for the opening relative to the long axis 1304, in one embodiment, is a patient-specific angle. Alternatively, or in addition, angle A for the opening relative to the long axis 1304, in one embodiment, is a patient-matched angle. In one embodiment, angle A (e.g., patient-specific angle or patient-matched angle) is defined at least in part based on medical imaging of a portion of a long bone of the patient (e.g., a tibia 226).

Advantageously, in one embodiment, the combined resection guide 1020 and fixation guide 1090 is patient-specific and the angle A between a long axis 1302 of the tibia and the orientation of the resection feature 1022 may be predefined and may be set to provide a surgeon with an optimal level of access and may be set based on the specific patient anatomy, conventional accepted practices, a surgeon's preferences or the like. Advantageously, angle A can direct a cutting tool to a superior surface of a talar dome of a talus 222 of the patient. In one embodiment, the angle A can be an angle A between about 5 degrees and about 80 degrees, or can be an angle A between about 10 degrees and 70 degrees, or can be an angle A between about 20 degrees and 60 degrees, or can be an angle A between about 30 degrees and 50 degrees, or can be an angle A between about 40 degrees and 45 degrees, or can be an angle A between about 45 degrees. In this manner, resection of the malleolus can provide access for remediation procedures on the talus 222.

In certain embodiments, a surgeon may perform one or more steps to facilitate a reduction of a bone segment of the medial malleolus to the tibia 226 after an osteotomy. Consequently, the surgeon may form bone tunnels, channels, and/or holes in the tibia 226 that can be used after the osteotomy to facilitate reducing a resected bone segment back on place after the osteotomy.

FIG. 13B, illustrates a stage of a surgical procedure in which a surgeon has deployed sleeves 1094 into the openings 1092 of the fixation guide 1090. The surgeon has also deployed fasteners 1010 through the sleeves 1094 and into the tibia 226.

Referring to FIGS. 13A and 13B, once a user positions the resection guide 1020 and fixation guide 1090 where desired, the user may secure the resection guide 1020 and fixation guide 1090 to the tibia 226 using one or more fasteners 1010. In certain embodiments, the fasteners 1010 may include a bend along the length of the fastener 1010. The bend may be part of the fastener 1010 initially or the user may create the bend after deploying a fastener 1010. The bend may serve to facilitate use of a cutting tool to perform an osteotomy of the tibia 226 using the resection feature 1022. The bend may prevent interference between a proximal end of the fastener 1010 and a cutting tool.

In the illustrated embodiment, the resection guide 1020 may include a separate structure that serves as a stop 1310. The stop 1310 serves to mitigate resection of patient tissue outside of a predefined area. In one embodiment, a stop 1310 is configured to control a maximum depth of a cutting tool inserted into a resection feature 1022. The stop 1310 is configured to interfere with insertion of a cutting implement of a cutting tool within the resection feature 1022 such that the cutting implement remains within the predefined area.

In particular, a stop 1310 can be used to manage a depth of a resection within the resection feature 1022. In certain embodiments, a stop 1310 includes a planar superior surface. Advantageously, the stop 1310 may also include a sloped or contoured surface such that the cutting implement can be inserted to a shallower depth or a deeper depth at different positions along an opening of a guide feature. Said another away a maximum depth can vary from one end of a resection feature 1022 (e.g., from an anterior end to a posterior end). Advantageously, the details of the shape and configuration of the stop 1310 can be predefined based on one or more of patient imaging data, anatomical structures of the patient, the osteotomy procedure being performed, preferences of the surgeon, a request of the surgeon, the nature of a patient's condition, and the like.

In certain embodiments, the stop 1310 is an extension of the body 1032. Alternatively, or in addition, rather than using an extension of the body structure as a stop, the resection guide 1020 may include a stop 1310 that includes one or more projections 1312a-b that extend from a surface of the body 1032 around one or more of the one or more resection feature 1022.

In one embodiment, not every resection feature 1022 may include projections 1312. In other embodiments, each one or more guide features 1022 may include one or more projections 1312. Advantageously, a height of one or more of the stops 1310 may be defined based on one or more of patient imaging data, anatomical structures of the patient, the osteotomy procedure being performed, preferences of the surgeon, a request of the surgeon, the nature of a patient's condition, and the like. Consequently, one resection feature 1022 may include a first maximum depth and another resection feature 1022 may include a second maximum depth.

By defining the resection guide 1020 to include one or more maximum depths for the one or more resection features 1022, the resection guide 1020 can assist a surgeon in performing an osteotomy in a way that mitigates a risk of the resections unintentionally damaging, or resecting, hard tissue or soft tissue other than what is intended for the surgical procedure. In one example, a surgeon may determine it is best to resect a malleolus of a distal tibia 226, but to stop the resection before contacting the talus 222. A stop 1310 enables a surgeon to manage the depth of the resection.

FIG. 13B illustrates an example embodiment of a resection guide 1020 that includes at least one resection feature 1022 and/or the body 1032 that includes a stop 1310. The stop 1310 may be defined to provide a maximum depth defined using patient imaging data. The stop 1310 is configured to limit the cutting tool 1320 to the maximum depth.

The stop 1310 in FIG. 13B is implemented using one or more projections 1312. At the stage of a procedure illustrated in FIG. 13B, a surgeon may begin by removing fasteners 1010 deployed using the fixation system 1094 so that the fastener 1010 do not interfere with a cutting tool. Next, a surgeon begins resecting tissue of the bone by inserting a blade 1322 of the cutting tool 1320, such as a surgical oscillating saw into a resection feature 1022. The blade 1322 can be inserted to the maximum depth and is stopped from further insertion into the resection feature 1022 by the stop 1310. Specifically, projection 1312 contacts a face 1324 of the cutting tool 1320 and prevents further insertion of the blade 1322.

Advantageously, a length of the blade 1322 (and/or a distance from a distal end of a blade to the face 1324) may be predefined, known, predetermined, and/or prescribed in a preoperative plan or prescription used for the surgical procedure. Advantageously, the resection guide 1020 with a stop 1310 can help a surgeon in performing the surgical procedure. If a surgeon resects until the cutting tool 1320 engages the stop 1310, the surgeon can be assured that the resection extends to a desired depth (not too far and not too short).

In certain embodiments, the body 1032 may not include projections 1312. Instead, the bends in the fasteners 1010 deployed near the resection feature 1022 may serve as a stop 1310. In such an embodiment, a user may insert the blade 1322 until a depth marking on the blade 1322 is about level with a bend in a fastener 1010. Alternatively, or in addition, a user may insert the blade 1322 until the face 1324 is about level with a bend in a fastener 1010. Alternatively, or in addition, a user may insert the blade 1322 until the face 1324 contacts a bend in a fastener 1010.

FIG. 13C, is a posterior view that illustrates a stage of the surgical procedure in which a surgeon has deployed sleeves 1094 into the openings 1092 of the fixation guide 1090 and has also deployed fasteners 1010 through the sleeves 1094 and into the tibia 226. The tibia 226 is shown transparent to illustrate that the fasteners 1010 can be inserted up to but not penetrating the lateral cortex of the tibia 226.

In certain embodiments, the fasteners 1010 may include markings on a side of them that indicates different depths of the fasteners 1010 during deployment. Advantageously, the present disclosure can provide the surgeon with instructions that indicate exactly how deep to insert the fasteners 1010 to get to a desired depth. The depth may be a recommended depth or one determined by the surgeon. FIG. 13C illustrates that the fasteners 1010 have been inserted deep enough to cross a cut plane that will be formed when the resection feature 1022 is used to resect the bone. By crossing the cut plane, a bone tunnel/opening on one side of the cut plane can be matched up to a bone tunnel/opening on an opposite side of the cut plane when a resected bone segment is reduced. The matched bone tunnels/openings facilitate reducing the resected malleolus to substantially the same position as before the resection.

The fasteners 1010 inserted through the openings 1092 can form bone tunnels/channels that can serve as guides when a resected bone segment of the tibia/medial malleolus is reduced. In one embodiment, the fasteners 1010 can be removed leaving bone tunnels/channels that can serve as guides when a resected bone segment of the tibia/medial malleolus is reduced. Alternatively, or in addition, cannulated screws and/or drills can be deployed over the fasteners 1010 to form bone tunnels/channels that can serve as guides when a resected bone segment of the tibia/medial malleolus is reduced. The bone tunnels/channels that can serve as pilot holes or pilot tunnels that can be used when reducing a bone segment in place on the distal end of the tibia.

FIG. 13C illustrates one example use of the bone attachment features 1024. In the illustrated embodiment, the resection guide 1020 includes two bone attachment features 1024. A bone attachment feature 1024 may be implemented in one embodiment by way of one or more holes 1056 that extend through the body 1032 from one side of the body 1032 to a bone-facing side of the body 1032 together with fasteners 1010.

In certain embodiments, the fasteners 1010 may be referred to as anchor pins because they serve to anchor the resection guide 1020 to a bone. In certain embodiments, the one or more holes 1056 have a circular cross-section and the cross-section has a first diameter substantially the same as a second diameter of one or more anchor pins configured for deployment through the one or more holes 1056 to secure the body 1032 to a long bone. Alternatively, or in addition, the holes 1056 and anchor pins (e.g., k-wires) may have a cross-section having a multi-sided shape (e.g., triangle, square, rectangle, pentagon, etc.).

In a bone attachment features 1024 that includes holes 1056 that extend through the body 1032, the angle or trajectory of the holes 1056 through the body 1032 can be defined to meet the needs of the user. In one embodiment, the angle or trajectory of the holes 1056 through the body 1032 is perpendicular to a surface upon which the hole originates. Alternatively, or in addition, the angle or trajectory of the holes 1056 through the body 1032 is at an angle A between about 90 degrees and about two degrees.

In one embodiment, the angle or trajectory of the holes 1056 through the body 1032 is fixed and the same for all embodiments of the resection guide 1020. A fixed angle or trajectory may work well in situations where the resection guide 1020 is used on a bone where the angle or trajectory is needed to properly anchor or secure the body 1032 but is not used for other purposes.

In another embodiment, the angle or trajectory of the holes 1056 through the body may be patient-specific. In another embodiment, the angle or trajectory of the holes 1056 through the body may be patient-matched. For example, in one osteotomy system 1000 the resection guide 1020 includes a trajectory guide. The trajectory guide serves to indicate for a user one or more trajectories for one or more aspects of a surgical procedure. For example, a trajectory guide may indicate a trajectory for fasteners 1010 that will be deployed in a bone attachment feature 1024. Alternatively, a trajectory guide may indicate a trajectory for fasteners 1010 that will be deployed using a fixation guide 1090.

Alternatively, a trajectory guide may indicate a trajectory for an osteotomy that is to be formed using a guide such as a resection feature 1022. For example, in the illustrated embodiment, the one or more holes 1056 of the bone attachment features 1024 extend through the body 1032 at a first trajectory (e.g., first angle relative to a long axis 1304) that substantially matches a second trajectory of an opening that serves as the resection feature 1022.

FIG. 13A includes a resection guide 1020 with a bone attachment features 1024 having holes 1056 that extend at trajectory 1302. Advantageously, the resection feature 1022 extends at substantially the same trajectory as trajectory 1302. FIG. 13C illustrates one example of a bone attachment feature 1024 that serves as a bone attachment feature 1024 and as a trajectory guide. In embodiments where the trajectory guide is patient-specific, an angle for fasteners 1010 of the bone attachment features 1024 and an angle for a resection feature 1022 may be defined and/or determined based on anatomic data 412, which may be derived from medical imaging data and/or from a bone model of a patient. In embodiments where the trajectory guide is patient-matched, an angle for fasteners 1010 of the bone attachment features 1024 and an angle for a resection feature 1022 may be defined and/or determined characteristics for a set of patients having common attributes.

FIG. 13C illustrates one advantage for a user with the configuration of FIGS. 13A and 13C (e.g., bone attachment features 1024 (also a trajectory guide) and resection feature 1022). The transparent tibia 226 illustrates the fasteners 1010 extending into the tibia 226 towards the distal end. Aligned with an anterior fastener 1010, the resection feature 1022 extends into the trajectory 1302 at this same angle. Thus, prior to resecting or dissecting bone of the tibia 226, a surgeon can visualize on a fluoroscopy display how and where the fasteners 1010 enter the tibia 226. Because the resection feature 1022 is at the same angle the user know that the cut or resection will follow the same path as the fastener 1010 into the bone. Thus, in this manner, the fastener 1010 telegraphs to a surgeon where the resection will go. With this information, a surgeon can decide whether to proceed with the surgical procedure or adjust some part of the procedure as desired in order to accomplish a desired outcome.

In another embodiment, the trajectory guide may be a structure separate from the bone attachment features 1024. For example, a clip (not shown) may extend from a body 1032 near the resection feature 1022. The clip may be configured to accept a fastener 1010 (e.g., K-wire) that can be snapped into the clip. The clip may be configured to hold the fastener 1010 in the same orientation and/or trajectory as the resection feature 1022. The fastener 1010 may engage the clip and be outside the skin of a patient or run alongside an outer surface of the tibia 226. Thus, when the fastener 1010 in the clip, is viewed under fluoroscopy the fastener 1010 indicates a trajectory for the resection feature 1022. Of course, a structure with a hole in it can be used in place of a clip. The position and orientation of the clip may be patient-specific or patient-matched.

Those of skill in the art will appreciate that the angle for the holes 1056 of a bone attachment features 1024 through the body 1032 relative to how a fastener 1010 enters the bone can be defined, managed, and/or determined in at least a couple of ways. First, the angle the fastener 1010 can be controlled by an angle of the hole 1056 through the body 1032. Since the body 1032 and fastener 1010 are both rigid, the fastener 1010 enters the bone at substantially the same angle as the hole 1056 through the body 1032.

Second, the angle of incidence for the fastener 1010 into a bone can be controlled by how the bone-facing (e.g., lateral side 1040) and/or bone-contacting side/surface of the body 1032 is configured. For example, if the bone-facing surface is planar and the hole 1056 extends through the body perpendicular to the bone-facing surface, then the fastener 1010 will enter the bone perpendicular to the bone surface. However, if the hole 1056 extends through the body perpendicular to the bone-facing surface and the bone-facing surface is angled at a first angle, then the fastener 1010 will enter the bone at a second angle complementary to the first angle. Thus, angling an opening where the fastener 1010 exist the body 1032 and enters bone can be used to control an angle of incidence for the fastener 1010 with the bone. This angle of incidence can be used to direct the fastener 1010 which can be used to show a user where an aligned resection feature 1022 will direct an osteotomy into the bone.

FIG. 13C also illustrates a side view of the sleeves 1094 and how the sleeves 1094 guide the fixation guide 1090 fasteners 1010 into the tibia 226. The transparent tibia 226 illustrates where the distal ends of the fasteners 1010 go into the tibia 226. The sleeve 1094 are received by openings 1092. The openings 1092 may extend into the fixation guide 1090 at an angle B. The angle B can be patient-matched and/or patient-specific. Alternatively, or in addition, the angle B can be a standard angle set based on where and/or how the fixation guide 1090 engages with a distal end (e.g., malleolus) of the tibia 226. In one embodiment, the openings 1092 of the fixation guide 1090 may extend through the fixation guide 1090 at a patient-specific angle. In one embodiment, the fasteners 1010 may include markings that indicate a depth of a fastener 1010 into the bone. The user may monitor progression of the fasteners 1010 into the bone until a desired depth is reached. The desired depth can be predefined in a preoperative plan. The desired depth may be set to ensure sufficient depth of the fastener 1010 without penetrating a lateral cortex of the tibia 226.

FIG. 13D, is an anterior view that illustrates a stage of the surgical procedure in which a surgeon has removed any fasteners 1010 deployed using the openings 1092, bone fasteners (e.g., bone screws), sleeves 1094 or the like from the openings 1092 of the fixation guide 1090. Advantageously, in this embodiment, the fixation guide 1090 has been used prior to performing an osteotomy using the resection feature 1022 to prepare for subsequent reduction and/or fixation. The preparations made for reduction and/or fixation can facilitate a successful union for the reduction.

The resection guide 1020 remains in place and a surgeon next uses a cutting tool to perform an osteotomy that separates the medial malleolus from the tibia 226. After the osteotomy, the surgeon can remove the combined resection guide 1020 and fixation guide 1090. Optionally, the surgeon can remove fasteners 1010 deployed for the bone attachment features 1024. In certain embodiments, the fasteners 1010 can be positioned such that the fasteners 1010 can remain in place after the osteotomy and can be used for steps of a reduction (e.g., compression and/or positioning).

FIG. 13E, is an anterior view that illustrates a stage of the surgical procedure in which a surgeon has removed the combined resection guide 1020 and fixation guide 1090. The fasteners 1010 may have also been remove, or are just not shown. FIG. 13E illustrates the path of the osteotomy guided by the resection guide 1020. Advantageously, the medial malleolus 1098 is separated from the tibia 226. The surgeon can next roll the separated medial malleolus 1098 in a medial and plantar direction to gain access to the talus 222. Advantageously, many of the soft tissues connected to and surrounding the medial malleolus 1098 can remain connected and can be retracted to provide clear access to the talus 222 for a remediation procedure on the talus 222.

FIG. 13F, is an anterior view that illustrates a stage of the surgical procedure in which a surgeon has reduced and fixated the medial malleolus 1098 to the tibia 226 following the remediation procedure on the talus 222. In one embodiment, the surgeon may redeploy the fasteners 1010 or new fasteners 1010 or other fasteners such as bone screws used prior to the osteotomy into holes or bone tunnels in the medial malleolus 1098 to ensure the medial malleolus 1098 is reduced substantially to its original position and orientation. The redeployed fasteners 1010 or alternative fasteners 1010 can be used as either temporary or permanent fixation to hold the reduced medial malleolus 1098 in place for healing.

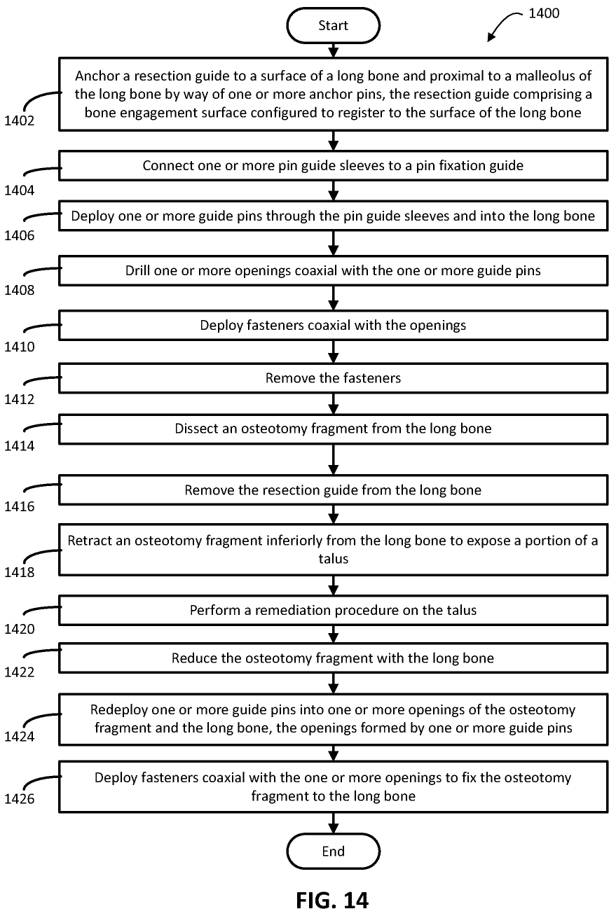

FIG. 14 illustrates a method for resecting and/or providing fixation for a bone portion to a tibia, according to one embodiment. FIG. 14 is a flowchart of an example process 1400. In some implementations, one or more process blocks of FIG. 14 may be performed by a device. In one embodiment, the process 1400 can be used for performing a malleolar osteotomy.

As shown in FIG. 14, a process 1400 may include anchoring 1402 a resection guide to a surface of a long bone and proximal to a malleolus of the long bone by way of one or more anchor pins, the resection guide having a bone engagement surface configured to register to the surface of the long bone (block 1402). In one embodiment, the malleolus is a medial malleolus. In another embodiment, the malleolus is a lateral malleolus. The long bone may be a tibia 226 or a fibula 228 or the like. For example, a user or a device may anchor a resection guide to a surface of a long bone and proximal to a medial malleolus of the long bone by way of one or more anchor pins, the resection guide having a bone engagement surface configured to register to the surface of the long bone, as described above. In one embodiment, the bone engagement surface is patient-specific. Alternatively, the bone engagement surface is patient-matched.

As also shown in FIG. 14, process 1400 may include connecting 1404 one or more pin guide sleeves to a pin fixation guide (e.g., fixation guide 1090) (block 1404). For example, a user or a device may connect one or more pin guide sleeves to a pin fixation guide, as described above. One example of a pin guide sleeve may be a sleeve 1094 as described above. One example pin fixation guide may be a fixation guide 1090 as described above. Connecting a pin fixation guide may involve sliding a sleeve 1094 into an opening 1092 of a fixation guide 1090.

As further shown in FIG. 14, process 1400 may include deploying 1406 one or more guide pins through the pin guide sleeves and into the long bone (block 1406). For example, a user or a device may deploy one or more guide pins through the pin guide sleeves and into the long bone, as described above. Examples of guide pins may include fasteners 1010 described above. In certain embodiments, deploying 1406 may include deploying a fastener 1010 until it crosses a planned cut plane for an osteotomy. Deploying 1406 one or more guide pins can form pilot tunnels/holes for use with subsequent fasteners to ensure that a resected bone fragment is aligned with another bone or bone fragment after reduction and an osteotomy.

As also shown in FIG. 14, process 1400 may include drilling 1408 one or more openings coaxial with the one or more guide pins (block 1408). For example, a user or a device may drill one or more openings coaxial with the one or more guide pins, as described above.

As further shown in FIG. 14, process 1400 may include deploying 1410 fasteners coaxial with the openings (block 1410). For example, a device may deploy fasteners coaxial with the openings, as described above. As also shown in FIG. 14, process 1400 may include removing 1412 the fasteners (block 1412). For example, a user or device may remove the fasteners, as described above. In certain embodiments, the fasteners may be cannulated bone screws that may be headed or headless.

In certain embodiments, the process 1400 steps of deploying 1406, drilling 1408, deploying 1410, and/or removing 1412 may be optional and certain steps may be omitted or substituted for others depending on how a surgeon wants to prepare the bone such that reduction of a resected malleolus can be readily reduced to substantially its original position before being resected/dissected in an osteotomy. For example, in one embodiment, the process 1400 can include deploying 1406 and drilling 1408 (in certain embodiments, drilling 1408 may not be needed if step deploying 1406 provides adequate guides for subsequent fixation) but the steps of deploying 1410 and removing 1412 may be omitted if a surgeon is satisfied that the openings formed by deploying 1406 and drilling 1408 will be sufficient to successfully reduce the malleolus after the osteotomy.

As further shown in FIG. 14, process 1400 may include dissecting 1414 an osteotomy fragment from the long bone (block 1414). For example, a user or a device may dissect a medial malleolus from the long bone, as described above. As also shown in FIG. 14, process 1400 may include removing 1416 the resection guide from the long bone (block 1416). For example, a user or a device may remove the resection guide from the long bone, as described above.

As further shown in FIG. 14, process 1400 may include retracting 1418 the osteotomy fragment inferiorly from the long bone to expose a portion of a talus (block 1418). In one embodiment, the osteotomy fragment includes a malleolus of the long bone. For example, a user or a device may retract the osteotomy fragment inferiorly from the long bone to expose a portion of a talus, as described above. The user may retract or dissect other soft tissue to retract the osteotomy fragment.

As also shown in FIG. 14, process 1400 may include performing 1420 a remediation procedure on the talus (block 1420). For example, a user or a device may perform a remediation procedure on the talus, as described above. One example of the remediation procedure may include an osteochondral autograft transplantation or osteochondral allograft transplantation.

As further shown in FIG. 14, process 1400 may include reducing 1422 the osteotomy fragment with the long bone (block 1422). For example, a user or a device may move the osteotomy fragment superiorly and back into its original position relative to the distal end of the tibia 226. Specifically, a proximal cut face of the osteotomy fragment may abut a distal cut face of the distal end of the tibia 226.

As also shown in FIG. 14, process 1400 may include redeploying 1424 one or more guide pins into the one or more openings of the osteotomy fragment and the long bone (block 1424). For example, a user or a device may redeploy the one or more guide pins into the one or more openings of the osteotomy fragment and the long bone, as described above. In certain embodiments, redeploying 1424 may be an optional step and instead step deploying 1426 fasteners may be taken and the fasteners may follow bone tunnels formed by guide pins and/or fasteners previously deployed.

As further shown in FIG. 14, process 1400 may include deploying 1426 fasteners coaxial with the one or more openings to fix the osteotomy fragment to the long bone (block 1426). For example, a user or a device may deploy fasteners coaxial with the one or more openings to fix the osteotomy fragment to the long bone, as described above. In certain embodiments, the fasteners may be cannulated bone screws that may be headed or headless. The fasteners could be new fasteners or the fasteners can be fasteners used earlier in the process 1400.

Although FIG. 14 shows example blocks of process 1400, in some implementations, process 1400 may include additional blocks, fewer blocks, different blocks, or differently arranged blocks than those depicted in FIG. 14. Additionally, or alternatively, two or more of the blocks of process 1400 may be performed in parallel.

Any methods disclosed herein comprise one or more steps or actions for performing the described method. The method steps and/or actions may be interchanged with one another. In other words, unless a specific order of steps or actions is required for proper operation of the embodiment, the order and/or use of specific steps and/or actions may be modified.

Reference throughout this specification to “an embodiment” or “the embodiment” means that a particular feature, structure or characteristic described in connection with that embodiment is included in at least one embodiment. Thus, the quoted phrases, or variations thereof, as recited throughout this specification are not necessarily all referring to the same embodiment.

Similarly, it should be appreciated that in the above description of embodiments, various features are sometimes grouped together in a single embodiment, Figure, or description thereof for the purpose of streamlining the disclosure. This method of disclosure, however, is not to be interpreted as reflecting an intention that any claim require more features than those expressly recited in that claim. Rather, as the following claims reflect, inventive aspects lie in a combination of fewer than all features of any single foregoing disclosed embodiment. Thus, the claims following this Detailed Description are hereby expressly incorporated into this Detailed Description, with each claim standing on its own as a separate embodiment. This disclosure includes all permutations of the independent claims with their dependent claims.

Recitation in the claims of the term “first” with respect to a feature or element does not necessarily imply the existence of a second or additional such feature or element. Elements recited in means-plus-function format are intended to be construed in accordance with 35 U.S.C. § 112 Para. 6. It will be apparent to those having skill in the art that changes may be made to the details of the above-described embodiments without departing from the underlying principles set forth herein.

While specific embodiments and applications of the present disclosure have been illustrated and described, it is to be understood that the scope of this disclosure is not limited to the precise configuration and components disclosed herein. Various modifications, changes, and variations which will be apparent to those skilled in the art may be made in the arrangement, operation, and details of the methods and systems of the present disclosure set forth herein without departing from it spirit and scope.

What is claimed is:

1. An apparatus for remediating a condition present in a patient, the apparatus comprising:
a body;
a bone engagement surface on a bone-facing side of the body, the bone engagement surface configured to engage a surface of a patient;
a bone attachment feature configured to couple the body to a long bone of the patient; and
a resection feature that guides resection of the long bone to separate a malleolus from the long bone.

2. The apparatus of claim 1, wherein the surface of the patient is a surface of the long bone and the bone engagement surface is shaped to match a contour of the surface of the long bone and defined at least in part based on medical imaging of a portion of the long bone.

3. The apparatus of claim 1, wherein the bone engagement surface is defined at least in part based on a bone model of a portion of the long bone.

4. The apparatus of claim 1, wherein:
the surface of the patient is a surface of skin covering the long bone of the patient; and
the resection feature is configured to guide resection of the skin by a cutting tool to expose the long bone.

5. The apparatus of claim 1, wherein the resection feature comprises an opening that extends from the bone-facing side to a side opposite the bone-facing side.

6. The apparatus of claim 5, wherein the opening of the resection feature extends through the body at a patient-specific angle defined at least in part based on medical imaging of a portion of the long bone of the patient.

7. The apparatus of claim 1, further comprising a patient-specific trajectory guide.

8. The apparatus of claim 1, further comprising a patient-matched trajectory guide.

9. The apparatus of claim 1, wherein the bone attachment feature comprises one or more holes that extend through the body from one side of the body to the bone-facing side of the body.

10. The apparatus of claim 9, wherein the one or more holes extend through the body at a first trajectory that substantially matches a second trajectory of an opening of the resection feature.

11. The apparatus of claim 9, wherein the one or more holes have a circular cross section and a first diameter substantially the same as a second diameter of one or more anchor pins configured for deployment through the one or more holes to secure the body to the long bone.

12. The apparatus of claim 1, further comprising a stop configured to control a maximum depth of a cutting tool inserted into the resection feature.

13. The apparatus of claim 1, further comprising a fixation guide having one or more openings that indicate a path for fasteners into the long bone.

14. An osteotomy system for remediating a condition present in a patient, comprising:
a resection guide comprising:
one or more bone attachment features configured to couple the resection guide to a bone; and
a resection feature that guides resection of the bone to dissect the bone into a proximal fragment and distal fragment; and
a fixation guide comprising one or more openings that indicate a path for fasteners through the distal fragment and into the proximal fragment of the bone.

15. The osteotomy system of claim 14, wherein the one or more bone attachment features align with the resection feature and one or more of the resection guide and the fixation guide comprises a bone engagement surface.

16. The osteotomy system of claim 14, wherein the fixation guide comprises one or more sleeves each configured to accept a fastener.

17. The osteotomy system of claim 14, further comprising a body having a coupler configured to couple the resection guide to the fixation guide and position the fixation guide relative to the resection guide.

18. The osteotomy system of claim 14, wherein the one or more openings of the fixation guide are aligned vertically with respect to a longitudinal axis of the bone.

19. The osteotomy system of claim 14, wherein the one or more openings of the fixation guide extend through the fixation guide at a patient-specific angle.

20. A method for performing a malleolar osteotomy, the method comprising:
anchoring a resection guide to a surface of a long bone and proximal to a malleolus of the long bone, the resection guide comprising a bone engagement surface configured to register to the surface of the long bone;
deploying one or more guide pins through the pin guide sleeves of a fixation guide and into the long bone;
deploying fasteners coaxial with the openings;
dissecting an osteotomy fragment from the long bone;
retracting the osteotomy fragment inferiorly from the long bone to expose a portion of a talus, the osteotomy fragment including the malleolus;
reducing the osteotomy fragment with the long bone;
redeploying one or more guide pins into the one or more openings of the osteotomy fragment and the long bone; and
deploying fasteners coaxial with the one or more openings to fix the osteotomy fragment to the long bone.
